SEQUENCE LISTING (19 sequences)

SEQ ID NO: 1 - protein, 288 aa, Artificial Sequence
  Synthetic N-terminal capping region
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, Artificial Sequence
  Synthetic C-terminal capping region
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 3 - protein, 7 aa, Simian virus 40
PKKKRKV

SEQ ID NO: 4 - protein, 10 aa, Simian virus 40
PKKKRKVEAS

SEQ ID NO: 5 - protein, 16 aa, Artificial Sequence
  Synthetic nucleoplasmin bipartite NLS
KRPAATKKAGQAKKKK

SEQ ID NO: 6 - protein, 9 aa, Homo sapiens
PAAKRVKLD

SEQ ID NO: 7 - protein, 11 aa, Homo sapiens
RQRRNELKRSP

SEQ ID NO: 8 - protein, 38 aa, Homo sapiens
NQSSNFGPMKGGNFGGRSSGPYGGGGQYFAKPRNQGGY

SEQ ID NO: 9 - protein, 42 aa, Homo sapiens
RMRIZFKNKGKDTAELRRRRVEVSVELRKAKKDEQILKRRNV

SEQ ID NO: 10 - protein, 8 aa, Homo sapiens
VSRKRPRP

SEQ ID NO: 11 - protein, 8 aa, Homo sapiens
PPKKARED

SEQ ID NO: 12 - protein, 8 aa, Homo sapiens
PQPKKKPL

SEQ ID NO: 13 - protein, 12 aa, Mus sp.
SALIKKKKKMAP

SEQ ID NO: 14 - protein, 5 aa, Influenza virus
DRLRR

SEQ ID NO: 15 - protein, 7 aa, Influenza virus
PKQKKRK

SEQ ID NO: 16 - protein, 10 aa, Hepatitis D virus
RKLKKKIKKL

SEQ ID NO: 17 - protein, 10 aa, Mus sp.
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

(19) United States
(12) Patent Application Publication
(10) Pub. No.: US 2022/0034870 A1
(43) Pub. Date: Feb. 3, 2022

(54) COMBINATORIAL CULTURE CONDITION ARRAYS AND USES THEREOF

(71) Applicant: The Broad Institute, Inc., Cambridge, MA (US)

(72) Inventors: Jesse Boehm, Cambridge, MA (US); Yuen-Yi Tseng, Cambridge, MA (US)

(21) Appl. No.: 17/387,548

(22) Filed: Jul. 28, 2021

Related U.S. Application Data
(60) Provisional application No. 63/057,812, filed on Jul. 28, 2020.

(51) Int. Cl. G01N 33/50; C12N 5/09; G06N 3/08

(52) U.S. Cl. CPC G01N 33/5044; G06N 3/08; C12N 5/0693

(57) ABSTRACT

Described in certain embodiments herein are combinatorial addressable arrays configured for high-throughput analysis of a sample and methods of using said combinatorial addressable arrays. Also described herein in certain embodiments are computer-implemented methods of training a statistical or machine learning model for determining and/or predicting culture conditions effective for growth of a biologic sample and computer-implemented method to determine and/or predict culture conditions effective growth for growth of a biologic sample.

### CROSS-REFERENCE TO RELATED APPLICATIONS

This application claims the benefit of and priority to co-pending U.S. Provisional Patent Application No. 63/057,812, filed on Jul. 28, 2020, entitled “COMBINATORIAL CULTURE CONDITION ARRAYS AND USES THEREOF”, the contents of which is incorporated by reference herein in its entirety.

### STATEMENT REGARDING FEDERALLY SPONSORED RESEARCH

This invention was made with government support under Grant No. HHSN2612015000031 awarded by the National Institutes of Health. The government has certain rights in the invention.

### SEQUENCE LISTING

This application contains a sequence listing filed in electronic form as an ASCII.txt file entitled BROD-4670US_ST25.txt, created on Jul. 27, 2021 and having a size of 7,853 bytes. The content of the sequence listing is incorporated herein in its entirety.

### TECHNICAL FIELD

The subject matter disclosed herein is generally directed to combinatorial arrays and their use for optimization of cell culture conditions.

### BACKGROUND

Cancer and other disease therapies, particularly heterogenous diseases and those whose presentation and symptoms are greatly influenced by the subject, can be difficult to understand and effectively treat at the individual patient level. Heterogeneity of the etiology and presentation of these diseases makes increases the importance of a personalized medicine approach for effective treatment for any given patient. Although increased understanding of the influence of patient specific factors, such as genetic and epigenetic background, has increased the resolution of patient stratification for many of these diseases, in most cases efficacies are only incrementally improved and still does not alleviate the issues disease heterogeneity presents.

In theory, one could sample, for example, a subject's tumor or tissue and determine which drug or drugs are most effective treating it, thus treating the patient and not the population. While elegantly simple in concept, it has still not been fully realized in practice, particularly for conditions and diseases where primary or differentiated cells are those needed to be cultured. For example, culturing cancer cells from solid tumors has historically not be rapid or readily feasible. Adding to this challenge, the material to work with may be relatively scant. For example, patients presenting with metastatic disease often undergo a needle biopsy rather than surgical resection, thus the amount of biopsy material to culture can be very limited. Given the difficult and limited nature of the material that is typically available, a robust personalized medicine approach to treatment is often unavailable due to the inability to meaningfully examine the diseased cells in vitro.

Thus, there exists a need for compositions, methods, and techniques for improving the in vitro culture success of cells and tissues that are challenging to culture in vitro and/or are of limited material.

Citation or identification of any document in this application is not an admission that such a document is available as prior art to the present invention.

### SUMMARY

Described in certain example embodiments herein are combinatorial addressable arrays configured for high-throughput analysis of a sample comprising: an addressable array configured to receive the sample and allocate the sample to a plurality of discrete locations across the addressable array, wherein two or more of the discrete locations of the addressable array comprises at least two different culture conditions, and wherein, for each of the at least two different culture conditions, there is at least two other discrete locations on the addressable array that each comprise only that culture condition.

In certain example embodiments, the at least two different culture conditions are each independently selected from the group consisting of: a culture media, a biological agent, a chemical agent, a pharmaceutical agent, a radioactive agent, a scaffold material, a culture type, a physical stress, a chemical stress, a biological stress, or any combination thereof.

In certain example embodiments, the cell culture media is a conditioned cell culture media.

In certain example embodiments, the two different culture conditions are each a cell culture media and wherein the cell culture medias are different from each other.

In certain example embodiments, one or both of the cell culture medias is/are a conditioned media.

In certain example embodiments, the condition media is conditioned media generated from a cancer cell line, a non-diseased cell line, a tumor organoid, a non-disease organoid, an engineered cell line, or any combination thereof.

In certain example embodiments, one or more of the discrete locations of the plurality of discrete locations on the addressable array comprises cells, tissue, an organoid, or any combination thereof.

In certain example embodiments, the cells, tissue, and/or organoid are cancer cells, cancer tissue, or cancer organoid or are generated from one or more cancer cells.

In certain example embodiments, the addressable array comprises a plurality of wells, one or more microfluidic channels, a two-dimensional (2D) polymer, a three-dimensional (3D) polymer, a gel, a planar surface, a non-planar surface, or any combination thereof.

Described in certain example embodiments here are high-throughput methods of empirically determining culture conditions effective to modify a biological sample, comprising: culturing a biological sample having an initial characteristic state in one or more of the discrete locations on the combinatorial addressable array of any of the preceding paragraphs and/or as described in other embodiments of the combinatorial addressable array elsewhere herein; and determining a change in the initial state of a characteristic of the biological sample, wherein the change in the initial state of the characteristic identifies one or more conditions effective to modify the characteristic in the biological sample.

In certain example embodiments, determining a change in the characteristic of the biological sample comprises performing gene and/or genome sequencing, a gene expression analysis, an epigenetic analysis, a cell phenotype analysis, a cell morphology analysis, a growth analysis, a differentiation analysis, a cell volume analysis, a cell viability analysis, a cell metabolism analysis, a cell communication or signal transduction analysis, a cell reproduction analysis, a cell response analysis, a cell production or secretion analysis, a cell function analysis or any combination thereof.

In certain example embodiments, the characteristic is growth, differentiation, proliferation, organoid formation, viability, death/apoptosis, cell product production and/or secretion, gene expression, protein expression, epigenome state, metabolism, cell volume, cell size, cell state, cell type or subtype, cell morphology, or any combination thereof.

In certain example embodiments, the biological sample comprises a cell or cell population, a tissue, an organoid, or any combination thereof.

In certain example embodiments, the cell population is a heterogenous or homogenous cell population.

In certain example embodiments, the biological sample comprises a cancer cell, a cancer tissue, a cancer organoid, or any combination thereof.

In certain example embodiments, the biological sample is cultured under 2D conditions, 3D conditions, suspension conditions, spheroid conditions, adherent conditions, aerobic conditions, anaerobic conditions, or any permissible combination thereof.

Described in certain example embodiments herein, are cell culture conditions effective to modify a characteristic of a biological sample during culture comprising: a cell culture condition identified by performing a method as in of any of the preceding paragraphs and/or as described in array elsewhere herein.

Described in certain example embodiments herein are methods of creating a cell line or organoid, the methods comprising: culturing a cell or cells isolated from a subject in one or more culture conditions of any of the preceding paragraph and/or described elsewhere herein; a combinatorial addressable array as in any of preceding paragraphs and/or described elsewhere herein; or any combination thereof.

In certain example embodiments, the population of cells forms an organoid, a spheroid, a cell suspension model, an adherent cell model, or any combination thereof.

In certain example embodiments, the cell or cells isolated from the subject is/are a cancer cell(s).

In certain example embodiments, culturing comprises passaging the cells one or more times.

In certain example embodiments, culturing does not comprise passaging.

In certain example embodiments, culturing comprises expanding the cells.

Described in certain example embodiments herein are computer-implemented methods of training a statistical or machine learning model for determining and/or predicting culture conditions effective for growth of a biologic sample, comprising: collecting a set of sample culture parameters from a database to generate a collected set of sample culture parameters; applying one or more transformations to each sample culture parameters to create a modified set of sample culture parameters; creating a first training set comprising the collected set of sample culture parameters, the modified set of sample culture parameters, and a set of non-effective sample culture parameter results; training a statistical model or machine learning algorithm in a first stage using the first training set; optionally creating a second training set for a second stage of training comprising the first training set and optionally, sample culture parameters that are incorrectly detected as effective sample culture parameters after the first stage of training; and optionally training the neural network in a second stage using the second training set.

In certain example embodiments, the database comprises one or more of the following: one or more clinical annotations of biologic samples, treatment response history of biologic samples, cell culture condition response and/or optimal parameters of/for biologic samples, processing method history of biologic samples, phenotype of biologic samples, genomic profile of biologic samples, epigenomic profile of biologic samples, and biologic sample source annotations.

In certain example embodiments, the one or more clinical annotations can be any one or more of those set forth in Appendix A of U.S. Provisional Application Ser. No. 63/057,812, which is incorporated by reference as if expressed in its entirety herein.

In certain example embodiments, the statistical model or machine learning algorithm is configured as a neural network, decision tree, support vector machine, linear regression, logistical regression, random forest, gradient boosted trees, naive bayes, nearest neighbor, k-means clustering, t-SNE, principal component analysis, association rule, Q-learning, temporal difference, Monte-Carlo tree search, asynchronous actor-critic agents, or any permissible combination thereof.

Described in certain example embodiments herein are computer-implemented methods for determining and/or predicting culture conditions effective for growth of a biologic sample, comprising: receiving biologic sample data; optionally applying one or more filters to the biologic sample data; using the received biologic sample data or filtered biologic sample data as input and applying a one or more classifiers to determine and/or predict one or more effective biologic sample biologic sample culture conditions based on a computer-accessible database, trained statistical or machine-learning model trained to predict effective biologic sample culture conditions based on the one or more classifiers, a statistical data analysis methodology, or any combination thereof.

In certain example embodiments, the one or more determined and/or predicted effective biologic sample culture conditions are passed through one or more additional filters to further optimize the determined and/or predicted effective biologic sample culture conditions.

In certain example embodiments, the method further comprises applying one or more additional classifiers to the one or more determined and/or predicted effective biologic sample culture conditions or further optimized determined and/or predicted effective culture conditions to determine and/or predict one or more effective biologic sample biologic sample culture conditions based on the computer-accessible database and/or trained machine-learning model trained to predict effective biologic sample culture conditions based on the one or more additional classifiers.

In certain example embodiments, the trained statistical or machine-learning model is produced by the method as in of any of the preceding paragraphs and/or as described elsewhere herein.

In certain example embodiments, the biologic sample data is received from user input, one or more sensors, one or more detection devices, one or more sample characteristic measurement and/or analysis devices, a database, or any combination thereof.

In certain example embodiments, the biological sample is contained in an addressable array as in any of the preceding paragraphs and/or as described elsewhere herein.

Described in certain example embodiments herein are computer-implemented methods to determine and/or predict culture conditions effective growth for growth of a biologic sample, comprising: receiving data of one or more parameters from the biologic sample in a format usable by a computing device; executing processing logic configured to generate feature data from the received data, filter the received data and/or the feature data, and/or process the feature data and/or received data with one or more trained machine learning models that is/are trained to predict effective biologic sample culture conditions based on the received data and/or feature data; and executing processing logic configured to cause a list of the effective biologic sample culture conditions to be displayed via an electronic display, transmitted to a user interface program, and/or be saved to a non-transitory computer readable memory.

In certain example embodiments, at least one of the one or more trained statistical or machine learning models are produced by the method as in any of the preceding paragraphs and/or as described elsewhere herein.

In certain example embodiments, the data of one or more parameters is received from user input, one or more sensors, one or more detection devices, one or more sample characteristic measurement and/or analysis devices, a database, or any combination thereof.

In certain example embodiments, the biological sample is contained in an addressable array as in any of the preceding paragraphs and/or as described elsewhere herein.

Described in certain example embodiments herein is non-transitory computer readable medium comprising computer-executable instructions recorded thereon for causing a computer to perform the method as in any of the preceding paragraphs and/or as described elsewhere herein.

Described in certain example embodiments herein are systems comprising non-transitory computer-readable medium; and a processor configured to execute instructions stored on the non-transitory computer readable medium which, when executed, cause the processor to perform the method as in any of the preceding paragraphs and/or as described elsewhere herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—General workflow comparison between conventional cell culture analysis and embodiments of a high-throughput combinatorial assay described herein.

FIGS. 2-3—Exemplary high-throughput combinatorial addressable array employing an empirical and dual media strategy. The high-throughput combinatorial addressable array can increase the success rate in identifying suitable culture conditions.

FIG. 4—Genomically confirmed rare tumor models generated using optimized culture conditions identified using embodiments of the high-throughput combinatorial addressable arrays and/or statistical and/or machine learning models described herein, which were then used to develop tumor models.

FIG. 5—Exemplary samples by tumor type developed using optimal culture conditions identified using embodiments of the high-throughput combinatorial addressable arrays and/or statistical and/or machine learning models described herein.

FIG. 6—Exemplary rare tumor models generated using optimized culture conditions identified using embodiments of the high-throughput combinatorial addressable arrays and/or statistical and/or machine learning models described herein, which were then used to develop tumor models.

FIG. 7—Different cell culture conditions can support propagation of different subclonal populations.

FIG. 8—Different cell culture conditions supported different desmoid tumors to grow.

FIG. 9—Model generation success broken down by tumor type.

FIG. 10—Exemplary development and collection of conditioned media from robust growing cell lines. Conditioned media can contain various bioactive factors (e.g. cytokines, growth factors, ECMs, etc.). In some embodiments, established cell line model collections, such as historical cancer cell lines and genetically engineered human/mouse cell lines, can be used to generate conditioned media.

FIG. 11—Tumor growth per diagnosis. Although some success has been achieved with some cell lines (some approaching 60% success rate), many still have less than a 5% growth success rate. This means there is much wasted efforts and resources on conditions and techniques that are not working for many cell types and patients. Further, in many cases, the samples are limited in material and thus such waste can severely hinder the success of treatment as without in vitro patient samples, then practitioners must rely on population outcomes (which may or may not apply) and in some cases random chance.

FIG. 12—Exemplary combinatorial addressable array that contains a matrix of different media types, that can optionally be selected using a trained statistical or machine learning model. This can reduce the cost, labor, and amount of sample needed while increasing the success rate of achieving viable cell growth, particularly for rare and difficult cell types.

FIG. 13—Machine prediction of tumor growth—Number of conditions per cell line after data cleaning. Information available to predict tumor growth were clinical annotations and culture conditions. The clinical annotations included tissue site, tumor type, and many others. The culture conditions tested were various (hundreds were tested) and included, for example, culture type (e.g., 2D or 3D), media type, etc. The HYBRID array/methodology required few samples to test many conditions (16 samples to test 64 different culture conditions) while the standard methodology required many samples to evaluate only 1-4 different culture conditions. Source of the data was divided by 1-4. Total raw data: 10,000 samples, 100 features (real time input in LIMS). Total cleaned data: 4500 samples, 14 features. Data was L-shaped. LIMS was used in conjunction with BSP and JIRA. Clinical features included cohort, diagnosis, primary disease, material type (e.g., fresh tissue, needle biopsy, blood, or cryopreserved), tissue site, tumor type (e.g., primary, metastasis, etc.), date and time of tumor collection. Culture condition features included flask coating, growth properties (e.g., 2D, 3D, and/or suspension), incubation condition (e.g., regular or hypoxia), media type (at initiation), starting media condition (native, 50/50 native/conditioned), 50/50 native/conditioned with supplementation)

FIG. 14—Model performance as demonstrated by ROC and Confusion Matrix.

FIG. 15—Rough Guide for classifying the accuracy of a diagnostic test based on the traditional academic point system. FIG. 15 shows three ROC curves representing excellent, good, and poor tests plotted on the same graph. The accuracy of the test depends on how well the test separates the group being tested into those with and without the disease or condition in question. Accuracy is measured by the area under the ROC curve. An area of 1 represents a perfect test. An area of 0.5 represents a poor test that does not provide any useful information.

FIG. 16—Machine prediction of tumor growth—Imbalanced Data.

FIG. 17—Machine prediction of tumor growth—Precision Recall Curve. AUC was about 70%. Precision was constant because recensions will always be the same weather you are classifying 10 or 1000 items.

FIG. 18—Screen shot of cell culture prediction algorithm tool clinical annotation input page. Clinical annotations can be input by a user and the statistical or trained learning algorithm will determine culture conditions based upon the clinical annotation and other inputs to provide recommended culture conditions for that particular sample.

FIG. 19—Screen shot of data output from the cell culture prediction algorithm.

FIG. 20—Model expansion optimization as exemplified by a paracrine support screen for LMS model propagation.

FIG. 21—Onboarding nine major cohorts to generate a rare cancer dependency map.

FIG. 22—HYBRID technology can reduce doubling time of OM established cell lines.

FIG. 23—Generation of a brain tumor model utilizing an embodiment of a high-throughput combinatorial addressable array and technique(s) as described herein and a neurosphere culture (e.g., medulloblastoma).

FIG. 24—Overview of the Cancer Cell Line Factory (CCLF) which has developed organoids, 2D cell lines and neurospheres as and for the development of patient models. CCLF has developed over 37 long-term genetically verified (p5-p20 and above), 100s of samples in flight. Currently, organoids represent 52% of lines developed, 2D cell lines represent about 37% of lines developed and neurospheres represent about 11% of cell lines developed.

FIG. 25—Steps in Developing a Rare Cancer Dependency Map.

FIG. 26—CRYO-Q workflow. CRYO-Q is a temporary cryopreservation queuing system for tumor cell model generation.

FIG. 27—Workflow of generating a model cell, tissue, or organoid line at CCLF. Success is considered when growing cells can be passaged at least 5 times with genomic verification.

FIG. 28—An embodiment of a computing machine 2000 and a module 2050 in accordance with certain example embodiments.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide combinatorial addressable arrays configured for high throughput analysis of a sample that can be capable of facilitating identification of optimal culture conditions of a sample. Embodiments disclosed herein also provide trained statistical and trained machine learning models that can identify optimal culture conditions using input from various resources including, without limitation, those from databases, user input information on the patient and sample, and characteristic data from a sample cultured on a combinatorial addressable array described herein. Embodiments disclosed herein provide methods of determining optimal culture conditions for a sample cultured on a combinatorial addressable arrays disclosed herein and/or using the trained statistical and/or trained machine learning model disclosed herein. The combinatorial addressable arrays herein can, particularly when used with a trained statistical or trained machine learning model described elsewhere herein, facilitate identification of optimal culture conditions more rapidly and from less sample material that conventional methods. This can allow for a personalized medicine approach for treating patients, particularly those who previously such an approach was not an option simply because the cells needed for in vitro analysis could not be readily cultured due to e.g., limited sample material and/or a lack of optimal culture conditions.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Combinatorial Addressable Arrays

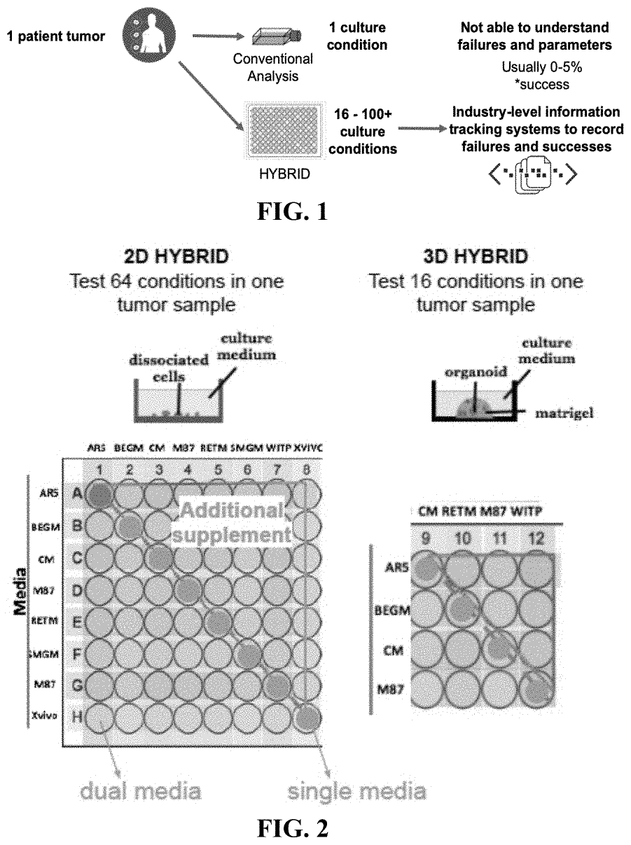

Described herein are embodiments of a combinatorial addressable array are configured for high-throughput analysis of a sample and can include an addressable array configured to receive the sample and allocate the sample to a plurality of discrete locations across the addressable array, where two or more of the discrete locations of the addressable array includes at least two different culture conditions, and where, for each of the at least two different culture conditions, there is at least two other discrete locations on the addressable array that each contain only that culture condition. FIG. 2 shows an exemplary embodiment of a combinatorial addressable array configured for high-throughput analysis of a sample and identifies example discrete locations that contain at least two different culture conditions and wells that contain one a single culture conditions which correspond to each of the at least two culture conditions.

### Combinatorial Addressable Arrays

As used herein, “array” encompasses any two or three dimensional ordered arrangement of features, where each feature has a unique position in two- or three-dimensional space. Thus, it will be appreciated that each feature in an array can be identified by a unique x,y (two-dimensional arrays) or unique x, y, z coordinate (three-dimensional arrays). Each feature of the array can be any physical, chemical, or biological, composition, property, or aspect that can or has the potential to bind with, react with, contain, fixate, incorporate, or otherwise hold in position a sample or a component thereof. As used herein, “addressable array” refers to an array where the unique position of each feature is predetermined and/or is organized such that each feature and/or its position is otherwise identifiable from each other feature and/or position thereof in the addressable array. Such predetermined and/or organized addressing of the features in an addressable array can allow for detection, measuring, determination, and/or identification of e.g., a specific target present in a sample, a specific sample characteristic(s), and/or response(s) present in a sample, a specific condition or set of conditions applied at each feature that elicits or causes a response in a sample, or any combination thereof, thus providing useable information about the sample or one or more component thereof and/or condition(s) applied to a sample.

Features can be arranged within an array (including an addressable array) such that there is substantially no distance between two or more features, that there is a distance between two or more features, or a combination thereof. In some embodiments, the distance between each feature is the same between each feature of the array. In some embodiments, the distance between each feature of the array can be varied. In some embodiments, the features can be contained in, attached to, integrated with, or otherwise coupled to a substrate or a surface thereof.

In some embodiments, one or more of the features can contain one or more sub features. The sub features can be contained in, attached to, integrated with, or otherwise coupled to the feature and/or substrate or a surface thereof. As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects. In some embodiments, the features can be adsorbed, physisorbed, or chemisorbed to a substrate. In some embodiments, the substrate can fix or hold the feature in a specific position within the array. In some embodiments, the features can be formed from voids present in the substrate (e.g., wells or etchings). In some embodiments, the sub features can be adsorbed, physisorbed, or chemisorbed to a substrate. In some embodiments, the substrate can fix or hold the sub feature in a specific position within the feature of the array. In some embodiments, the sub features can be formed from voids present in a feature (e.g., void, engraving or etching). Sub features can be arranged within a feature of the array such that there is substantially no distance between two or more sub features, that there is a distance between two or more sub features, or a combination thereof. In some embodiments, the distance between each sub feature is the same between each sub feature. In some embodiments, the distance between each sub feature of the array can be varied. In some embodiments, the sub features can be contained in, attached to, integrated with, or otherwise coupled to a feature, the substrate and/or a surface thereof.

Further aspects of the array are discussed in greater detail elsewhere herein.

### Array Substrate

The substrate can be solid, vitreous solid, semisolid, liquid, gel, hydrogel, or any permissible combination thereof. As used herein, “hydrogel” refers to a gelatinous colloid, or aggregate of polymeric molecules in a finely dispersed semi-solid state, where the polymeric molecules are in the external or dispersion phase and water (or an aqueous solution) forms the internal or dispersed phase. Generally, hydrogels are at least 90% by weight of an aqueous solution. The substrate can be any permissible shape or size. In some embodiments the substrate can be or have a regular shape. In some embodiments the substrate can have an irregular shape. The substrate can have any useful form including beads, bottles, planar objects (e.g., slides, plates, etc.), matrices, containers, vessels, dishes, fibers, wafers, plates (e.g., single well plates, multi-well plates, etched, engraved, etc.), chips, membranes, particles, microparticles, sticks, strips, thin films, tapes, fibers, tubes, chambers, droplets, capillaries, or any combination thereof.

A substrate can contain a single array or can contain multiple arrays. In some embodiments, a substrate can contain a single addressable array. In some embodiments, the substrate can contain multiple addressable arrays.

In some embodiments, one or more dimensions of the substrate (e.g., a length, a width, a height, a diameter, and the like) can range from about 1-1,000 pm, nm, μm, cm, or mm. In some embodiments, one or more dimensions of the substrate can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the largest dimension of the substrate can range from 1-1,000 pm, nm, μm, cm, or mm. In some embodiments, the largest dimension of the substrate can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the smallest dimension of the substrate can range from 1-1,000 pm, nm, μm, cm, or mm. In some embodiments, the smallest dimension of the substrate can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments, the substrate can have a volume. The volume of the substrate can range from about 1-1,000 pm³, nm³, μm³, cm³, mm³, or L³. In some embodiments, the substrate volume can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments, the features are attached or otherwise coupled on one or more surfaces of the substrate. As used herein, “surface,” in the context herein, refers to a boundary of an object, such as the substrate. The surface can be an interior surface (e.g., the interior boundary of a hollow object), or an exterior or outer boundary of a substrate. Generally, the surface of a substrate corresponds to the idealized surface of a three dimensional solid that is topological homeomorphic with the substrate. The surface can be an exterior surface or an interior surface. An exterior surface forms the outermost layer of a substrate or device. An interior surface surrounds an inner cavity of a substrate or device, such as the inner cavity of a tube. As an example, both the outside surface of a tube and the inside surface of a tube are part of the surface of the tube. In some embodiments, one or more surfaces can be modified with one or more features. In some embodiments, one or more surfaces can be functionalized to facilitate attachment or coupling of one or more features to the surface.

In some embodiments, one or more dimensions of the surface (e.g., a length, a width, a height, a diameter, and the like) can range from about 1-1,000 pm, nm, μm, cm, or mm. In some embodiments, one or more dimensions of the surface is/are about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the largest dimension of the surface ranges from about 1-1,000 pm, nm, μm, cm, or mm. In some embodiments, the largest dimension of the surface can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the smallest dimension of the surface ranges from about 1-1,000 pm, nm, μ m, cm, or mm. In some embodiments, the smallest dimension of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments the surface area of the surface ranges from about 1-1,000 pm², nm², μm², cm², or mm². In some embodiments, the surface area of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm², nm², μm², cm², or mm².

In some embodiments, the surface has a volume. In some embodiments, the volume of the surface ranges from about 1-1,000 pm³, nm³, μm³, cm³, mm³, or L³. In some embodiments, the surface volume is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments, the substrate has a volume. In some embodiments, the volume of the substrate ranges from about 1-1,000 pm³, nm³, μm³, cm³, mm³, or L³. In some embodiments, the substrate volume is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments the surface and/or substrate can be porous. In some embodiments the pores of the surface and/or substrate can be substantially homogenous. In some embodiments the pores of the surface and/or substrate can be heterogenous. Pores can have any irregular or regular shape. In some embodiments the surface and/or substrate a population of pores can have an average diameter, average largest dimension, and/or average smallest dimension that can range from 1-1,000 pm, nm, μm, cm, or mm. In some embodiments, the average diameter, average largest dimension, and/or average smallest dimension of the a population of pores is/are about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments, one or more pores of the substrate and/or surface has a diameter, a largest dimension, and/or a smallest dimension that ranges from about 1-1,000 pm, nm, μm, cm, or mm. In some embodiments one or more pores of the substrate and/or surface has a diameter, a largest dimension, and/or a smallest dimension that is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments, the population of pores of the substrate and/or surface has a total pore volume. In some embodiments, the total pore volume of the substrate and/or surface ranges from about 1-1,000 pm³, nm³, μm³, cm³, mm³, or L³. In some embodiments, the total poor volume is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments, all or one or more parts of the substrate and/or surface is/are opaque. In some embodiments, all or one or more parts of the substrate and/or surface is/are transparent. In some embodiments, all or one or more parts of the substrate and/or surface is/are semi-transparent.

The substrate and/or surface can be completely composed of or include any suitable material(s). Suitable materials include, but are not limited to, glass, ceramics, polymers, gels, hydrogels, adhesives, metals, metalloids, metal alloys, non-metals, crystals, fibrous material, and combinations thereof. The substrate and/or surface can be composed of a biocompatible material.

The term “biocompatible”, as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

Biocompatibility, as used herein, can be quantified using the following in vivo biocompatibility assay. A material or product is considered biocompatible if it produces, in a test of biocompatibility related to immune system reaction, less than 50%, 45%, 40%, 35%, 30%, 25%, 20%, 15%, 10%, 8%, 6%, 5%, 4%, 3%, 2%, or 1% of the reaction, in the same test of biocompatibility, produced by a material or product the same as the test material or product except for a lack of the surface modification on the test material or product. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be a three-dimensional network (e.g., the repeat units are linked together left and right, front and back, up and down), a two-dimensional network (e.g., the repeat units are linked together left, right, up, and down in a sheet form), or a one-dimensional network (e.g., the repeat units are linked left and right to form a chain). “Polymers” can be composed, natural monomers or synthetic monomers and combinations thereof. The polymers can be biologic (e.g., the monomers are biologically important (e.g., an amino acid), natural, or synthetic. As used interchangeably herein, “polymer blend” and “polymer mixture” refers to a macroscopically homogenous mixture of two or more different species of polymers. Unlike a copolymer, where the monomeric polymers are covalently linked, the constituents of a “polymer blend” and “polymer mixture” are separable by physical means and does not require covalent bonds to be broken. A “polymer blend” can have two or more (e.g., 2, 3, 4, 5, 6, 7, 8, 9, 10 or more) different polymer constituents.

Exemplary synthetic polymers include, without limitation, poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

As used herein, “glass” refers to any type of glass including, but not limited to silicate glasses (e.g., soda-lime glass, borosilicate glass, lead glass, aluminosilicate glass, glass-ceramics, and fiber glass), silica-free glasses (e.g., amorphous metals and polymers), and molecular liquids and molten salts. Glasses can contain additives that can modify e.g., the optical properties (e.g., transparency, color, refractivity etc.), conductive properties or other properties of the glass.

As used herein, “metal” refers to Li, Be, Na, Mg, Al, K, Ca, Sc, Ti, V, Cr, Mn, Fe, Co, Ni, Cu, Zn, Ga, Rb, Sr, Y, Zr, Nb, Mo, Tc, Ru, Rh, Pd, Ag, Cd, In, Sn, Cs, Ba, La, Ce, Pr, Nd, Pm, Sm, Eu, Gd, Tb, Dy, Ho, Er, Rm, Yb, Lu, Hf, W, Re, Os, Ir, Pt, Au, Hg, Tl, Pb, Bi, Po, Ra, Ac, Th, Pa, U, Np, Am, Cm, Bk, Cf, Es, Fm, Md, No, Lr, Rf, Db, Sg, Bh, Hs, Mt, Ds, Rg, Cn, Nh, Fl, Mc, Lv, and combinations thereof. As used herein, “metalloid” refers to B, Si, Ge, As, Sb, Te, At, and combinations thereof. As used herein, “non-metal” refers to He, H, C, N, O, F, Ne, P, S, Cl, Ar, Se, Br, Kr, I, Xe, Rn, and combinations thereof.

As used herein, “fibrous material” refers to any bulk material composed of a plurality of fibers. The fibers the fibrous material can be composed of glass, biological polymers (e.g., proteins, polynucleotides), metals, metalloids, non-metals, carbon nanostructures, polymers, crystals, ceramics, metal alloys, and combinations thereof. The fibers can be formed of natural or synthetic materials. The fibrous material can form any usable form, such as a sheet, membrane, strip, tape, slide, fiber, mesh, and the like. The fibrous material can be a flexible, semi-flexible, or inflexible material. Generally, fibrous materials where the individual fibers are loosely coupled to or associated with each other will be more flexible than those where the individual fibers are more tightly coupled or associated with each other. Exemplary fibrous materials include, but are not limited to paper sheets, paper strips and paper tapes, polymeric membranes, fabrics, and fibrous glass membranes.

In some embodiments, all or one or more parts of the surface and/or the substrate can be hydrophilic. In some embodiments, all or one or more parts of the surface and/or the substrate can be hydrophobic. In some embodiments, all or one or more parts of the surface and/or substrate can be superhydrophobic. In some embodiments, patterns on the surface and/or substrate can be formed by specific placement of hydrophobic and/or hydrophilic materials. In some embodiments, such patterns can, without limitation, form features of the array and/or form conduits to provide sample, reactants, features, and the like to one or more regions of the array. As used herein, “hydrophilic”, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the molecule is considered hydrophilic. As used herein, “hydrophobic”, refers to molecules which have a greater affinity for, or solubility in an organic solvent as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the molecule is considered hydrophobic. In some embodiments, hydrophobic and hydrophilic regions can be formed by particular materials that are hydrophobic or hydrophilic or can be formed by changing the texture of a surface (e.g., by etching, scoring, etc.) such that the contact angle or other interaction of water or liquid with the surface is changed such that that region such that it is hydrophobic or hydrophilic.

In some embodiments, the suitable material can be a hydrophobic material. Suitable hydrophobic materials include, but are not limited to: acrylics (e.g., acrylic, acrylonitrile, acrylamide, and maleic anhydride polymers), polyamides and polyimides, carbonates (e.g., Bisphenol A-based carbonates), polydienes, polyesters, polyethers, polyfluorocarbons, polyolefins (e.g., polyethylene, polypropylene, and copolymers thereof), polystyrenes and copolymers thereof, polyvinyl acetals, polyvinyl chlorides and polyvinylidene chlorides, poly vinyl ethers and polyvinyl ketones, polyvinylpyridines and polyvinypyrrolidones, Aculon's Transition Metal Complex coting, SLIPS coating material (Adaptive Surface Technologies), and any combination thereof.

In some embodiments, the suitable material can be composed of or include a superhydrophobic material. Suitable superhydrophobic materials include, but are not limited to manganese oxide polystyrene, zinc oxide polystyrene, precipitated calcium carbonate, carbon nanotubes, silica nano-coatings, fluorinated silanes, and flurophopolymer coatings. See e.g., Meng et al. 2008, The Journal of Physical Chemistry C. 112 (30): 11454-11458; Hu et al. 2009. Colloids and Surfaces A: Physicochemical and Engineering Aspects. 351 (1-3): 65-70; Lin et al., Colloids and Surfaces A: Physicochemical and Engineering Aspects. 421: 51-62; Das et al., RSC Advances. 4 (98): 54989-54997. doi:10.1039/C4RA10171E; Torun et al., 2018. Macromolecules. 51 (23): 10011-10020; Warsinger et al. 2015, Colloids and Surfaces A: Physicochemical and Engineering Aspects. 421: 51-62; Servi et al. 2017, Journal of Membrane Science. Elsevier BV. 523: 470-479

In some embodiments, the suitable material can be composed of or include a hydrophilic material. Hydrophilic materials include, but are not limited to, hydrophilic polymers such as poly(N-vinyl lactams), poly(vinylpyrrolidone), poly(ethylene oxide), poly(propylene oxide), polyacrylamides, cellulosics, methyl cellulose, polyanhydrides, polyacrylic acids, polyvinyl alcohols, polyvinyl ethers, alkylphenol ethoxylates, complex polyol monoesters, polyoxyethylene esters of oleic acid, polyoxyethylene sorbitan esters of oleic acid, and sorbitan esters of fatty acids; inorganic hydrophilic materials such as inorganic oxide, gold, zeolite, and diamond-like carbon; and surfactants such as Triton X-100, Tween, Sodium dodecyl sulfate (SDS), ammonium lauryl sulfate, alkyl sulfate salts, sodium lauryl ether sulfate (SLES), alkyl benzene sulfonate, soaps, fatty acid salts, cetyl trimethylammonium bromide (CTAB) a.k.a. hexadecyl trimethyl ammonium bromide, alkyltrimethylammonium salts, cetylpyridinium chloride (CPC), polyethoxylated tallow amine (POEA), benzalkonium chloride (BAC), benzethonium chloride (BZT), dodecyl betaine, dodecyl dimethylamine oxide, cocamidopropyl betaine, coco ampho glycinate alkyl poly(ethylene oxide), copolymers of poly(ethylene oxide) and poly(propylene oxide) (commercially called Poloxamers or Poloxamines), alkyl polyglucosides, fatty alcohols, cocamide MEA, cocamide DEA, cocamide TEA, Adhesives Research (AR) tape 90128, AR tape 90469, AR tape 90368, AR tape 90119, AR tape 92276, and AR tape 90741 (Adhesives Research, Inc., Glen Rock, Pa.). Examples of hydrophilic film include, but are not limited to, Vistex® and Visguard® films from (Film Specialties Inc., Hillsborough, N.J.), and Lexan HPFAF (GE Plastics, Pittsfield, Mass.). Other hydrophilic surfaces are available from Surmodics, Inc. (Eden Prairie, Minn.), Biocoat Inc. (Horsham, Pa.), Advanced Surface Technology (Billerica, Mass.), and Hydromer, Inc. (Branchburg, N.J.) and any combination thereof. Surfactants can be mixed with reaction polymers such as polyurethanes and epoxies to serve as a hydrophilic coating.

In some embodiments, the suitable material can be composed of or include a conductive and/or magnetic material. Conductive materials include, without limitation, metals, electrolytes, superconductors, semiconductors and some nonmetallic conductors such as graphite and conductive polymers. Magnetic materials include without limitation, any magnetic material including those that are ferromagnetic, paramagnetic and diamagnetic. In some embodiments, the magnetic material can include those that are electromagnetic (i.e., those materials that become magnetic or become a more powerful magnet when an electric current is applied to them). Exemplary magnetic materials include, but are not limited to, iron, nickel, cobalt, steel, rare earth metals (e.g., gadolinium, samarium, and neodymium), and combinations thereof.

In some embodiments, the suitable material can be composed or include an electric insulator material. Exemplary electric insulator materials include, but are not limited to, rubber, glass, oil, air, diamond, dry wood, dry cotton, plastic, fiberglass, porcelain, ceramics and quartz.

In some embodiments, the surface of the substrate is made out of the same material as the substrate and is essentially integrated and indistinguishable from the substrate. In some embodiments, the surface is made out of a different material as the substrate. In some embodiments the surface is essentially a coating, film, or layer present on at least part of or the entirety of the substrate and is thus readily distinguishable from the substrate.

### Array Features

As previously described the array can have one or more features. In some embodiments, one or more of the features can have sub-features. In some embodiments, the sub features themselves can form an array within the feature (or also referred to herein as a sub array).

The number of features can range from 1 to 100, 1,000, 10,000 or more. In some embodiments, the number of features is 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 or more.

The number of sub features can range from 1 to 100, 1,000, 10,000 or more. In some embodiments, the number of sub features is 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 15000, 20000, 25000, 30000, 35000, 40000, 45000, 50000 or more.

In some embodiments the features and/or sub features can be wells (including but not limited to, microwells, nanowells, picowells, etc.), capillaries, microcapillaries, nanocapillaries, droplets, beads, oligonucleotides, polynucleotides, antibodies, affibodies, aptamers, polypeptide:polynucleotide complexes, gel forms, hydrogel forms, columns, matrices, and any permissible combinations thereof.

In some embodiments the features and/or sub features can hold a volume ranging from 1-1,000 pm³, nm³, μm³, cm³, mm³, or L³. In some embodiments, the wells, microwells, and/or nanowells capillaries, microcapillaries, nanocapillaries, and/or other areas formed on a surface of a substrate can hold a volume can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments, one or more dimensions of the features and/or sub features (e.g., a length, a width, a height, a diameter, and/or the like) ranges from about 1-1,000 pm, nm, μm, cm, or mm. In some embodiments, one or more dimensions of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the largest dimension of the features and/or sub features ranges from 1-1,000 pm, nm, μm, cm, or mm. In some embodiments, the largest dimension of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the smallest dimension of the features and/or sub features ranges from about 1-1,000 pm, nm, μm, cm, or mm. In some embodiments, the smallest dimension of the surface is about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

The features can be any container, region, area, droplet, vessel, and the like capable of containing a volume of fluid. In some of such embodiments, the features are wells, (including but not limited to, microwells, nanowells, picowells, etc.) capillaries, microcapillaries, nanocapillaries, and/or other areas formed on a surface of a substrate. The wells, microwells, and/or nanowells capillaries, microcapillaries, nanocapillaries, and/or other areas formed on a surface can be any regular or irregular 2D or 3D shape. In some embodiments, all the wells, microwells, and/or nanowells capillaries, microcapillaries, nanocapillaries, and/or other areas formed on a surface are homogenous. In some embodiments, all the wells, microwells, and/or nanowells capillaries, microcapillaries, nanocapillaries, and/or other areas formed on a surface are heterogenous.

In some embodiments the features, (e.g., wells, capillaries, microcapillaries, nanocapillaries, and/or other areas formed on a surface of a substrate can have a surface capable of holding a fluid) can include or be composed of a cell scaffold material and/or a material that facilitates cell adherence to a surface. Exemplary cell scaffold materials include, but are not limited to Matrigel, collagen and other extracellular matrix components, decellularized tissue, polysaccharides (e.g., alginate, chitosan, cellulose, dextran, chitin, glycosaminoglycan, hyaluronic acid, agarose and combinations thereof), polymers, ceramics, any material set forth in Nikolova and Chavali (2019. Bioact Mater. 4:271-292), particularly e.g., Tables 1-3 and Sections 3-6, and any combination thereof.

### Culture Conditions

The combinatorial addressable array described herein can be organized such that combinations of culture conditions can be tested on a sample. In some embodiments, the at least two different culture conditions are each independently selected from the group of: a culture media, a biological agent, a chemical agent, a pharmaceutical agent, a gene modifying agent, a radioactive agent, a scaffold material, a culture type, a physical stress, a chemical stress, a biological stress, or any combination thereof. In some embodiments, 3, 4, 5, 6, 7, 8, 9, 10, 11, 12, 13, 14, 15, 16, 17, 18, 19, 20 or more different conditions are tested on a given sample. In some embodiments, each sample condition tested is independently selected from a culture media, a biological agent, a chemical agent, a pharmaceutical agent, a gene modifying agent, a radioactive agent, a scaffold material, a culture type, a physical stress, a chemical stress, a biological stress, or any combination thereof.

In some embodiments, the two different culture conditions are each a cell culture media and wherein the cell culture medias are different from each other. In some embodiments, one or both of the cell culture medias is/are a conditioned media.

### Culture Media

The sample(s) can be cultured in a cell/tissue culture medium. The cell medium can be a natural cell culture medium. The cell medium can be an artificial cell culture media. The term “natural cell culture medium” as used herein, refers to cell culture media that is composed of only naturally occurring biological fluids in their native condition (i.e., not supplemented or mixed). Such biological fluids include, but are not limited to, plasma, lymph, placental cord serum, and amniotic fluid. The cell medium can be an artificial cell culture medium. As used herein, “artificial cell culture medium” refers to cell culture media that is not a natural media and can be synthetically constructed by, for example, supplementation of nutrients, vitamins, salts, gases (e.g., CO₂, O₂) or mixing natural cell culture media together. Artificial media can include balanced salt solutions, basal media (e.g., MEM, DMEM), and complex media (e.g., PRMI-1640, and IMDM).

It will be appreciated that where a specific media is provided herein, that further supplementation can occur so as to evaluate the effect of the condition within the array.

In some embodiments, the cell culture medium is serum free. In some embodiments, the cell culture medium contain serum (either natural or supplemented (e.g., fetal bovine serum, human serum, horse serum, and the like).

In some embodiments, the cell culture medium is protein free. Example protein free media include, but are not limited to, MEM and RPMI-1640. In some embodiments, the cell culture medium contains one or more proteins and/or peptides. Exemplary and non-limited proteins/peptides include albumin, transferrin, aprotinin, fetuin, fibronectin.

The cell culture medium can contain one or more fatty acids and/or lipids. Lipids which may be used include, but are not limited to, the following classes of lipids: fatty acids and derivatives, mono-, di and triglycerides, phospholipids, sphingolipids, cholesterol and steroid derivatives, terpenes and vitamins. Fatty acids and derivatives thereof may include, but are not limited to, saturated and unsaturated fatty acids, odd and even number fatty acids, cis and trans isomers, and fatty acid derivatives including alcohols, esters, anhydrides, hydroxy fatty acids and prostaglandins. Saturated and unsaturated fatty acids that may be used include, but are not limited to, molecules that have between 12 carbon atoms and 22 carbon atoms in either linear or branched form. Examples of saturated fatty acids that may be used include, but are not limited to, lauric, myristic, palmitic, and stearic acids. Examples of unsaturated fatty acids that may be used include, but are not limited to, lauric, physeteric, myristoleic, palmitoleic, petroselinic, and oleic acids. Examples of branched fatty acids that may be used include, but are not limited to, isolauric, isomyristic, isopalmitic, and isostearic acids and isoprenoids. Fatty acid derivatives include 12-(((7′-diethylaminocoumarin-3yl)carbonyl)methylamino)-octadecanoic acid; N-[12-(((7′diethylaminocoumarin-3-yl)carbonyl)methyl-amino)octadecanoyl]-2-aminopalmitic acid, N succinyl-dioleoylphosphatidylethanol amine and palmitoyl-homocysteine; and/or combinations thereof. Mono, di and triglycerides or derivatives thereof that may be used include, but are not limited to, molecules that have fatty acids or mixtures of fatty acids between 6 and 24 carbon atoms, digalactosyldiglyceride, 1,2-dioleoyl-sn-glycerol; 1,2-cdipalmitoyl-sn-3 succinylglycerol; and 1,3-dipalmitoyl-2-succinylglycerol.

Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophosphoethanolamine. Synthetic phospholipids with asymmetric acyl chains (e.g., with one acyl chain of 6 carbons and another acyl chain of 12 carbons) may also be used.

Sphingolipids which may be used include ceramides, sphingomyelins, cerebrosides, gangliosides, sulfatides and lysosulfatides. Examples of Sphinglolipids include, but are not limited to, the gangliosides GM1 and GM2

Steroids which may be used include, but are not limited to, cholesterol, cholesterol sulfate, cholesterol hemi succinate, 6-(5-cholesterol 3β-yloxy) hexyl6-amino-6-deoxy-1-thio-α-D-galactopyranoside, 6-(5-cholesten-3β-tloxy)hexyl-6-amino-6-deoxyl-1-thio-α-D mannopyranoside and cholesteryl)4′-trimethyl 35 ammonio)butanoate.

Additional lipid compounds which may be used include tocopherol and derivatives, and oils and derivatized oils such as stearylamine.

The cell culture medium can contain one or more amino acids. In some embodiments, one or more of the amino acids is provided in L form. In some embodiments, one or more of the amino acids is provided in D form. One or more of the amino acids can be essential amino acids. One or more of the amino acids can be non-essential amino acids.

The cell culture medium can contain one or more carbohydrate sources, such as glucose maltose, galactose, fructose and combinations thereof.

The cell culture medium can contain one or more salts. In some embodiments, the salt can be an inorganic salt. The salt composition and concentration can be altered as desired and can optionally be included in the media outside of a physiological range so as to apply another stressor or another condition to be analyzed by the array.

The cell culture medium can contain one or more vitamins.

The cell culture medium can contain one or more minerals. Exemplary minerals include, but are not limited to, calcium, phosphorus, magnesium, sodium, potassium, chloride, sulfur, iron, manganese, copper, iodine, zinc, cobalt, selenium, and molybdenum.

The cell culture medium can contain one or more anti-infectives. As used herein, “anti-infective” refers to compounds or molecules that can either kill an infectious agent or inhibit it from spreading. Anti-infectives include, but are not limited to, antibiotics, antibacterials, antifungals, antivirals, and antiprotozoals.

The cell culture medium can contain one or more buffers or buffer systems. The buffer or buffer system can act to achieve a desired pH of the cell culture medium. This may or may not be a physiologic pH. It will be appreciated that the buffer or buffer system can therefore add a different condition to the cell—pH as previously discussed. Exemplary and non-limiting buffers can include gaseous buffers such as CO₂, which balances with the CO₃/HCO₃ content of the cell culture medium. Chemical buffering systems can include zwitterions, for example HEPES. Others are generally known in the art.

The cell culture medium can contain a suitable pH indicator, such as phenol red, which can allow for constant monitoring of the pH. It will be appreciated that other pH indicators may be suitable and, in some cases, be preferred as phenol red can mimic the action of some steroid hormones, such as estrogen, in some cells and can, in some case interfere with sodium-potassium homeostasis. It will be appreciated that that later can be neutralized, by the inclusion of serum or bovine pituitary hormone. Also, if the samples or other aspect of the array is to be analyzed using flow cytometric studies, then it can be desirable to use a pH indicator other than phenol red.

The cell culture medium can contain an active agent. As used herein, “active agent” or “active ingredient” refers to a substance, compound, or molecule, which is biologically active or otherwise, induces or is capable of inducing a biological or physiological effect on a subject, which can include cells and tissues, to which it is administered to. In other words, “active agent” or “active ingredient” refers to a component or components of a composition to which the whole or part of the effect of the composition is attributed. Active agents are described here and elsewhere herein.

### Conditioned Media

In some embodiments, the sample can be cultured in conditioned media at one or more features of the array. As used herein “conditioned media” refers a medium in which a specific cell or cell population have been cultured in, and then separated from the medium by removal of the medium or removal of the cells. While the cells were cultured in the medium, they secrete cellular factors that include, but are not limited to hormones, cytokines, chemokines, neurotrophins, extracellular matrix (ECM), proteins, vesicles (including but not limited to exosomes), antibodies, granules and combinations thereof. The medium plus the secreted cellular factors composes the conditioned medium. In some embodiments, the condition media is conditioned media generated from a diseased cell line, a cancer cell line, a non-diseased cell line, a tumor organoid, a non-disease organoid, an engineered cell line, or a combination thereof.

Exemplary and non-limiting growth factors that can be secreted and thus contained in conditioned media include vascular endothelial growth factor (VEGF), bone morphogenetic protein(s) (BMP), a transforming growth factor (TGF) such as transforming growth factor beta, a platelet derived growth factor (PDGF), an epidermal growth factor (EGF), a nerve growth factor (NGF), an insulin-like growth factor (e.g., insulin-like growth factor I), scatter factor/hepatocyte growth factor (HGF), granulocyte/macrophage colony stimulating factor (GMCSF), a glial growth factor (GGF), and a fibroblast growth factor (FGF), GCSF, Erythropoietin, TPO, GDF, neurotrophins, MSF, SGF, GDF, activin, CTGF, Epigen, Galectin, KGF, leptin, MMIF, MIA (melanoma inhibitory activity), myostatin, noggin, NOV, omentin, Oncostatin-M, Osteopontin, OPG, periostin, placental growth factor, placental lactogen, prolactin, RNAK ligand, retinol binding protein (RBP), stem cell factor, amphiregulin, lymphocyte function associated Antigen-3, myeloid derived growth factor, osteoclast stimulating factor, progranulin, colony stimulating factor and combinations thereof.

Exemplary and non-limiting cytokines that can be secreted and thus contained in conditioned media include 4-1BB, adiponectin, AITR, AIF1, B-cell activating factor, beta defensin, betacellulin, BMP, BST1, B type Natrriuretic peptide, cardiotrophin, CTLA4, EBI3, Endoglin, epiregulin, FAS, Flt3 ligand, follistatin, hedgehog protein, interferons (e.g. interferon alpha, interferon gamma, interferon tau, interferon beta, interferon regulatory factor), interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-8, IL-19, IL-20, IL-21, IL-22, IL-23, IL-24, IL-27, IL-28A, IL-29, IL-31, IL-32, IL-33, IL-34, IL-35, IL-36, IL-37), otoraplin, resistin, leukemia inhibitory factor, serum amyloid A, TPO, trefoil factor, thymic stromal lymphopoietin, tumor necrosis factor, uteroglobin, visfatin, wingless-type MMTV nitration site family, AIMP1, CLCF1, CYTL1, EMAP II, TAFA2, Vaspin, and combinations thereof.

Exemplary and non-limiting chemokines that can be secreted and thus contained in conditioned media include CXCL13, CXCL14, CCL6, CCL27, CXCL16, CXCL17, CXCL6, CXCL5, eotaxin, CCL2, CX3CL1, CXCL1, 2, 3, CCL14, CCL1, CXCL8, CXCL11, CC3L1, XCL1, CCL2, 7, 8, 12, 13, CCL22, CCL28, CXCL9, CCL3, 4, 9, 15, CXCL7, CCL4, CXCL4, CXCL12, CCL17, CCL25, CCL16, FAM19A5, CXCL15, and combinations thereof.

Exemplary and non-limiting hormones that can be secreted and thus contained in conditioned media include endothelin, exendin, follicle stimulating hormone, growth hormone releasing hormone, growth hormone releasing peptides, ipamorelin, glucagon, glucagon-like peptides, insulin, chorionic gonadotropin, inhibin-Beta C Chain, inhibin alpha, inhibin alpha chain, luteinizing hormone, luteinizing hormone releasing hormone, peptide hormones (e.g., adrenocorticotropic hormone, alarelin, antide, atosiban, buserelin, cetrorelix, desmopressin, deslorelin, elcatonin, ganirelx, ghrelin, goserelin, hexarelin, gistrelin, lanreotide, leuprolide, lypressin, melanotan-I and -II, nafarelin, octreotide, pramlintide, secretin, sincalide, somatostatin, terlipressin, thymopentin, triptorelin, vasopressin, neuropeptide Y, cholecystokinin), procalcitonin, prolactin, oxytocin, parathyroid hormone, estrogen, testosterone, stanniocalcin-1 and -2, thymosin, thyrostimulin, thyroid stimulating hormone, agouti-related protein, calcitonin, corticotrophin releasing hormone binding protein, prouroguanylin, oxyntomodulin, thyrotripin releasing hormone, and combinations thereof.

In some embodiments, the conditioned media can be prepared from a reference, known, and/or otherwise characterized cell line and the profile of one or more cellular factors in the conditioned media is known. Exemplary and non-limited reference, known, or otherwise characterized cell lines that can be used, in some embodiments, to produce conditioned media for use in the combinatorial assay described herein include primary cells. In some embodiments the reference primary cell line can be a reference diseased primary cell line. In some embodiments, the reference primary cell line can be a normal cell line. The reference cells can be, without limitation, muscle, heart, lung, blood vessels, bone, liver, kidney, brain, neuronal, glial, astrocyte, heart, pituitary, adrenal gland, thyroid, immune, skin, pancreas, intestinal, stomach, esophageal, fat, or corneal cells or a combination thereof. In some embodiments, the cells are tumor cells. Many such lines are known in the art. Exemplary lines include without limitation:

(a) Primary Epithelial Cells such as Primary Small Airway Epithelial Cells Fibrosis (ATCC® PCS-301-014), Primary Small Airway Epithelial Cells; COPD (ATCC® PCS-301-013), Primary Small Airway Epithelial Cells; Asthma (ATCC® PCS-301-011), Primary Bronchial/Tracheal Epithelial Cells; Fibrosis (ATCC® PCS-300-014), Primary Bronchial/Tracheal Epithelial Cells; COPD (ATCC® PCS-300-013), Primary Bronchial/Tracheal Epithelial Cells; Asthma (ATCC® PCS-300-011);

(b) Primary Fibroblasts such as Primary Lung Fibroblast; Fibrosis (ATCC® PCS-201-020), Primary Lung Fibroblasts; COPD (ATCC® PCS-201-017), Primary Lung Fibroblasts; Asthma (ATCC® PCS-201-015), Primary Lung Fibroblasts; Cystic Fibrosis (ATCC® PCS-201-016); Primary Dermal Fibroblast; Normal, Human, Adult (HDFa) (ATCC® PCS-201-012); Primary Uterine Fibroblast Cells; Normal, Human (HUF) (ATCC® PCS-460-010); Primary Bladder Fibroblast Cells; Normal, Human (ATCC® PCS-420-013); Primary Gingival Fibroblast; Normal, Human, Adult (HGF) (ATCC® PCS-201-018); Primary Lung Fibroblasts; Asthma (ATCC® PCS-201-015);

(c) primary endothelial cells such as Primary Coronary Artery Endothelial Cells; Normal, Human (HCAEC) (ATCC® PCS-100-020), Primary Umbilical Vein Endothelial Cells; Normal, Human (HUVEC) (ATCC® PCS-100-010), Primary Umbilical Vein Endothelial Cells; Normal, Human, Pooled (HUVEC) (ATCC® PCS-100-013), Primary Pulmonary Artery Endothelial Cells; Normal, Human (HPAEC) (ATCC¬Æ PCS-100-022), Primary Aortic Endothelial Cells; Normal, Human (HAEC) (ATCC® PCS-100-011), Primary Dermal Microvascular Endothelial Cells; Normal, Human, Neonatal (HDMVECn) (ATCC® PCS-110-010)),

(d) primary epithelial cells such as Primary Bladder Fibroblast Cells; Normal, Human (ATCC® PCS-420-013), Primary Bladder Smooth Muscle Cells; Normal, Human (HBdSMC) (ATCC® PCS-420-012™), Primary Bladder Epithelial Cells (A/T/N); Normal, Human (BdEC) (ATCC® PCS-420-010™), Primary Bronchial/Tracheal Smooth Muscle Cells; Normal, Human (ATCC® PCS-130-011), Primary Small Airway Epithelial Cells; Fibrosis (ATCC PCS-301-014), Primary Small Airway Epithelial Cells; COPD (ATCC® PCS-301-013); Primary Bronchial/Tracheal Epithelial Cells; Fibrosis (ATCC® PCS-300-014); Primary Bronchial/Tracheal Epithelial Cells; Fibrosis (ATCC® PCS-300-014); Primary Lobar Epithelial Cells (ATCC® PCS-300-015); Primary Bronchial/Tracheal Epithelial Cells; Asthma (ATCC® PCS-300-011); Primary Corneal Epithelial Cells; Normal, Human (ATCC® PCS-700-010); Primary Mammary Epithelial Cells; Normal, Human (HMEC) (ATCC® PCS-600-010); Primary Prostate Epithelial Cells; Normal, Human (HPrEC) (ATCC® PCS-440-010); Primary Renal Mixed Epithelial Cells; Normal, Human (HREC) (ATCC® PCS-400-012); Primary Renal Cortical Epithelial Cells; Normal, Human (HRCE) (ATCC® PCS-400-011); Primary Renal Proximal Tubule Epithelial Cells; Normal, Human (RPTEC) (ATCC® PCS-400-010); Primary Cervical Epithelial Cells (ATCC® PCS-480-011); Primary Vaginal Epithelial Cells (ATCC® PCS-480-010);

(e) keratinocytes such as Primary Epidermal Keratinocytes; Normal, Human, Adult (HEKa) (ATCC® PCS-200-011); Primary Gingival Keratinocytes (ATCC® PCS-200-014); Primary Epidermal Keratinocytes; Normal, Human, Neonatal Foreskin (HEKn) (ATCC® PCS-200-010)

(f) stem cells such as Primary Bone Marrow CD34+ Cells, Normal, Human (ATCC® PCS-800-012); Adipose-Derived Mesenchymal Stem Cells; Normal, Human (ATCC® PCS-500-011); Bone Marrow-Derived Mesenchymal Stem Cells; Normal, Human (ATCC® PCS-500-012); Umbilical Cord-Derived Mesenchymal Stem Cells; Normal, Human (ATCC® PCS-500-010); ATCC-HYS0103 Human Induced Pluripotent Stem (IPS) Cells (ATCC® ACS-1020); ATCC-BYS0110 Human [African American Male] Induced Pluripotent Stem (IPS) Cells (ATCC® ACS-1024); ATCC-DYP0730 Human Induced Pluripotent Stem (IPS) Cells (ATCC® ACS-1003); ATCC-DYR0530 Human Induced Pluripotent Stem (IPS) Cells (ATCC® ACS-1012); Neural Progenitor Cells Derived from XCL-1 GFAPp-Nanoluc-Halotag (ATCC® ACS-5006); Neuronal Progenitor Cells Derived from XCL-1 MAP2p-Nanoluc-Halotag (ATCC® ACS-5007); Neural Progenitor Cells Derived from ATCC-DYS0530 Parkinson's Disease (ATCC® ACS-5001)

(g) immune cells such as Primary Bone Marrow Mononuclear Cells, Normal, Human (BMMC) (ATCC® PCS-800-013); Primary Peripheral Blood CD14+ Monocytes, Normal, Human (ATCC® PCS-800-010); Primary CD56+ NK Cells (ATCC® PCS-800-019); Primary CD19+ B Cells (ATCC® PCS-800-018); Primary CD8+ Cytotoxic T Cells (ATCC® PCS-800-017); Primary CD4+ Helper T Cells (ATCC® PCS-800-016); Primary Peripheral Blood Mononuclear Cells (PBMC), Normal, Human (ATCC® PCS-800-011);

(f) muscle cells such as Primary Bronchial/Tracheal Smooth Muscle Cells; Normal, Human (ATCC® PCS-130-011); Primary Lung Smooth Muscle Cells; Normal, Human (ATCC® PCS-130-010); Primary Aortic Smooth Muscle Cells; Normal, Human (HASMC) (ATCC® PCS-100-012); Primary Bladder Smooth Muscle Cells; Normal, Human (HBdSMC) (ATCC® PCS-420-012); Primary Coronary Artery Smooth Muscle Cells; Normal, Human (HCASMC) (ATCC® PCS-100-021); Primary Pulmonary Artery Smooth Muscle Cells; Normal, Human (PASMC) (ATCC® PCS-100-023); Primary Uterine Smooth Muscle Cells; Normal, Human (HUtSMC) (ATCC® PCS-460-011);

(h) tumor/cancers cells such as any cell line available in the Cancer Cell Line Encyclopedia (available at https://portals.broadinstitute.org/ccle); Bladder Cancer Cell Panel (ATCC® TCP-1020); Bone Cancer Panel (ATCC® TCP-1009); Glioma Tumor Cell Panel (ATCC® TCP-1018); Brain Cancer Cell Panel (ATCC® TCP-1017); Triple-Negative Breast Cancer Panel 3 (ATCC® TCP-1003); Triple-Negative Breast Cancer Panel 2; Mesenchymal & Luminal Morphology (ATCC® TCP-1002); Breast Cancer Biomarkers Cell Line Panel 1 (ATCC® TCP-1004); ATCC Breast Cancer Cell Panel (ATCC® 30-4500K); Triple-Negative Breast Cancer Panel 1; Basal-Like Morphology (ATCC® TCP-1001); Breast Cancer Mouse Model Cell Line Panel (ATCC® TCP-1005); Colon Cancer Panel 2, BRAF (ATCC® TCP-1007); Colon Cancer Panel 1, KRAS (ATCC® TCP-1006); Ovarian Cancer Panel (ATCC® TCP-1021); Uterine Cancer Cell Panel (ATCC® TCP-1023); Gynecological Cancer Cell Panel (ATCC® TCP-1024); Cervical Cancer Cell Panel (ATCC® TCP-1022); Head and Neck Cancer Panel (ATCC® TCP-1012); Leukemia p53 Hotspot Mutation Cell Panel (ATCC® TCP-2070); BCL-2 Family Cell Panel 1 (ATCC® TCP-2100); Lung Cancer Panel (ATCC® TCP-1016); Small Cell Lung Cancer p53 Hotspot Mutation Cell Panel (ATCC® TCP-2040); Non-Small Cell Lung Cancer p53 Hotspot Mutation Cell Panel (ATCC® TCP-2030); Melanoma Cancer Cell Panel (ATCC® TCP-1013); Metastatic Melanoma Cancer Cell Panel (ATCC® TCP-1014); Pancreatic Cancer p53 Hotspot Mutation Cell Panel (ATCC® TCP-2060); Pancreatic Cancer Panel (ATCC® TCP-1026); Soft-Tissue Sarcoma Cell Panel (ATCC® TCP-1019); Stomach (Gastric) Cancer Panel (ATCC® TCP-1008); PTEN Genetic Alteration Cell Panel (ATCC® TCP-1030); ERK Genetic Alteration Cell Panel (ATCC® TCP-1033); RAS Genetic Alteration Cell Panel (ATCC® TCP-1031); MET Genetic Alteration Cell Panel (ATCC® TCP-1036); EGFR Genetic Alteration Cell Panel (ATCC® TCP-1027); PI3K Genetic Alteration Cell Panel (ATCC® TCP-1028); FGFR Genetic Alteration Cell Panel (ATCC® TCP-1034); BRAF Genetic Alteration Cell Panel (ATCC® TCP-1032); AKT Genetic Alteration Cell Panel (ATCC® TCP-1029);

(i) kidney cells such as HEK cells and variants (OAT1 HEK 293T cells, HEK-293, HEK-293T, etc.), mIMCD-3, CV-1/EBNA, MDCK, WT 9-12, WT 9-7, Caki-2, 786-O, 769-P, A-498 cells, Phoenix-ECO, HCM-BROD-0051-C64, ACHN, Phoenix-AMPHO, PEAKrapid, HK-2. G-401, A-704, Caki-1, G-402, Hs 926.T, SK-NEP-1, WSS-1;

(j) liver cells such as Huh-7, HepG2, Hep 3B, HTB-79, HTB-52, CRL-2064, CRL-1837, CRL-5822, CRL-2235, CRL-2238, CRL-2234, CRL-2236, CRL-2237, CRL-10741, CRL-11997, CRL-2233, HB-8064, HB-8065, CRL-8024, CRL-5892, CRL-5987, CRL-2706, CRL-11233;

(k) neural/brain cells such as CRL-8621, CRL-2137m CRL-2142, CRL-2149, CRL-2266, CRL-2267, CRL-2927, CRL10742, CRL-2885, CRL-2886, CRL-3245, CRL-2020, CRL-2365, CRL2366, CRL-7899, CRL-8621, CRL-3304; CRL-3021, CRL-3034, HTB-186, HTB-187, HTB-185, CRL-3408, CRL-3409, CRL-3410, CRL-3411, CRL-3412, CRL-3413, CRL-3414, CRL-3415, CRL-3416, CRL-3417, ACS-1018;

(l) eye/retinal cells such as WERI-Rb-1, Y79, APRE-19, APRE-19/HPV-16;

(m) heart cells such as HL-1, AC16;

(n) fat cells such as Lisa, LS-14, AML-1, SGBS, Chub-S7;

(o) muscle cells such as MD, A-673, Hs 792(C).M, SJCRH30, SW 684, A-204, Hs 729, T/G HA-VSMC, Hs 235.Sk;

(p) bladder cells such as SCaBER, TCCSUP, T24, RT4, J82, UM-UC-3, HT-1197, 5637, HT-1367, SW 780;

(q) prostate cells such as PNT2, WPE1-NB26-64, RWPE-1, RWPE-2, PC-3, WPMY-1, CA-HPV-10, C4-2B, PC-3-Luc-2, MDA PCa 2b, LASCPC-01, C4-2, C4, VCaP, NCI-H660, DU 145, 22Rv1, PWR-1E, PZ-HPV-7, WPE1-NB11, WPE1-NB14, WPE1-NA22, WPE-stem;

(r) bone cells such as K7M2 wt, SK-ES-1, SJSA1, Hs 822.T, Hs 888.T, SaOs2, MG-63, MC3T3E1; and/or

(s) combinations thereof.

### Active Agents

In some embodiments, the culture condition can be or include one or more active agents, such as a biologic agent(s), chemical agent(s), pharmaceutical agent(s), gene modifying agent(s), radioactive agent(s), or a combination thereof.

In some embodiments the active agent can be a biologic agent. As used herein, “biologic agent” refers to any compound, composition, biopolymer, molecule and the like that is made by a living organism and include, without limitation, polynucleotides (e.g. DNA, RNA), peptides and polypeptides, and chemical compounds (e.g. hormones, chemokines, and cytokines). In some embodiments, the biologic agent can be an antibody or fragment thereof. As used herein, “antibody” refers to a protein or glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen. “Antibody” includes single valent, bivalent and multivalent antibodies.

As used herein, “deoxyribonucleic acid (DNA)” and “ribonucleic acid (RNA)” generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

In some embodiments, the biologic agent is one that is secreted by a cell. Such substances are discussed in greater detail herein with respect to conditioned media. It will be appreciated that such biologic agents can be provided to a sample in the form of conditioned media or be collected from a cell or cell media, separated or purified from the cell or cell media, and provided to the sample.

In some embodiments, the active agent is a chemical agent. As used herein, “chemical agent” refers to a chemical substance, molecule, or composition. Exemplary chemical agents are those that are suitable for use as a pharmaceutical agents in an animal as well as those at are not. In some embodiments, the chemical agent is a hazardous chemical agent. In some embodiments, the chemical agent is not hazardous. In some embodiments, the chemical agent can be a carcinogen. In some embodiments, the chemical agent is biocompatible. The term “biocompatible”, as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

In some embodiments, the active agent can be a pharmaceutical agent. As used herein, “pharmaceutical agent” refers to any compound, molecule, or composition that is capable of preventing, treating, diagnosing, and/or prognosing a disease, condition, disorder, or any symptom thereof. Pharmaceutical agents can be of any type, including without limitation chemical agents and biologic agents.

In some embodiments, the active agent(s) is/are a growth factor(s). Exemplary growth factors include without limitation vascular endothelial growth factor (VEGF), bone morphogenetic protein(s) (BMP), a transforming growth factor (TGF) such as transforming growth factor beta, a platelet derived growth factor (PDGF), an epidermal growth factor (EGF), a nerve growth factor (NGF), an insulin-like growth factor (e.g., insulin-like growth factor I), scatter factor/hepatocyte growth factor (HGF), granulocyte/macrophage colony stimulating factor (GMCSF), a glial growth factor (GGF), and a fibroblast growth factor (FGF), GCSF, Erythropoietin, TPO, GDF, neurotrophins, MSF, SGF, GDF, activin, CTGF, Epigen, Galectin, KGF, leptin, MMIF, MIA (melanoma inhibitory activity), myostatin, noggin, NOV, omentin, Oncostatin-M, Osteopontin, OPG, periostin, placental growth factor, placental lactogen, prolactin, RNAK ligand, retinol binding protein (RBP), stem cell factor, amphiregulin, lymphocyte function associated Antigen-3, myeloid derived growth factor, osteoclast stimulating factor, progranulin, colony stimulating factor and combinations thereof.

In some embodiments, the active agent(s) is/are a cytokine(s). Exemplary cytokines include without limitation cytokines that can be secreted and thus contained in conditioned media include 4-1BB, adiponectin, AITR, AIF1, B-cell activating factor, beta defensin, betacellulin, BMP, BST1, B type Natrriuretic peptide, cardiotrophin, CTLA4, EBI3, Endoglin, epiregulin, FAS, Flt3 ligand, follistatin, hedgehog protein, interferons (e.g. interferon alpha, interferon gamma, interferon tau, interferon beta, interferon regulatory factor), interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-8, IL-19, IL-20, IL-21, IL-22, IL-23, IL-24, IL-27, IL-28A, IL-29, IL-31, IL-32, IL-33, IL-34, IL-35, IL-36, IL-37), otoraplin, resistin, leukemia inhibitory factor, serum amyloid A, TPO, trefoil factor, thymic stromal lymphopoietin, tumor necrosis factor, uteroglobin, visfatin, wingless-type MMTV nitration site family, AIMP1, CLCF1, CYTL1, EMAP II, TAFA2, Vaspin, and combinations thereof.

In some embodiments, the active agent(s) is/are a chemokine(s). Exemplary chemokines include without limitation CXCL13, CXCL14, CCL6, CCL27, CXCL16, CXCL17, CXCL6, CXCL5, eotaxin, CCL2, CX3CL1, CXCL1, 2, 3, CCL14, CCL1, CXCL8, CXCL11, CC3L1, XCL1, CCL2, 7, 8, 12, 13, CCL22, CCL28, CXCL9, CCL3, 4, 9, 15, CXCL7, CCL4, CXCL4, CXCL12, CCL17, CCL25, CCL16, FAM19A5, CXCL15, and combinations thereof.

In some embodiments, the active agent(s) is/are a hormone(s). Exemplary growth include without limitation endothelin, exendin, follicle stimulating hormone, growth hormone releasing hormone, growth hormone releasing peptides, ipamorelin, glucagon, glucagon-like peptides, insulin, chorionic gonadotropin, inhibin-Beta C Chain, inhibin alpha, inhibin alpha chain, luteinizing hormone, luteinizing hormone releasing hormone, peptide hormones (e.g., adrenocorticotropic hormone, alarelin, antide, atosiban, buserelin, cetrorelix, desmopressin, deslorelin, elcatonin, ganirelx, ghrelin, goserelin, hexarelin, gistrelin, lanreotide, leuprolide, lypressin, melanotan-I and -II, nafarelin, octreotide, pramlintide, secretin, sincalide, somatostatin, terlipressin, thymopentin, triptorelin, vasopressin, neuropeptide Y, cholecystokinin), procalcitonin, prolactin, oxytocin, parathyroid hormone, steroid hormones (e.g., estradiol, testosterone, tetrahydro testosterone, estrogen), stanniocalcin-1 and -2, thymosin, thyrostimulin, thyroid stimulating hormone, agouti-related protein, calcitonin, corticotrophin releasing hormone binding protein, prouroguanylin, oxyntomodulin, thyrotripin releasing hormone, eiconsanoids (e.g., arachidonic acid, lipoxins, and prostaglandins), and combinations thereof.

In some embodiments, the active agent(s) is/are other immunomodulatory agents(s). Exemplary other immunomodulatory agents include without limitation prednisone, azathioprine, 6-MP, cyclosporine, tacrolimus, methotrexate, antibodies, glucans, aptamers, and combinations thereof.

In some embodiments, the active agent(s) is/are an antipyretic(s). Exemplary antipyretics include without limitation non-steroidal anti-inflammatories (e.g., ibuprofen, naproxen, ketoprofen, nimesulide, diclofenac, diflunisal, etodolac, indomethacin, ketorolac, nabumetone, oxaprozin, piroxicam, salsalate, sulindac, tolmetin, celecoxib, valdecoxib, firocoxib, and rofecoxib), aspirin and related salicylates (e.g., choline salicylate, magnesium salicylate, and sodium salicylate), paracetamol/acetaminophen, metamizole, phenazone, and quinine.

In some embodiments, the active agent(s) is/are an anxiolytic(s). Exemplary anxiolytics include without limitation benzodiazepines (e.g. alprazolam, bromazepam, chlordiazepoxide, clonazepam, clorazepate, diazepam, flurazepam, lorazepam, oxazepam, temazepam, triazolam, and tofisopam), serotonergic antidepressants (e.g. selective serotonin reuptake inhibitors, serotonin-norepinephrine reuptake inhibitors, tricyclic antidepressants (e.g. imipramine, doxepin, amitriptyline, nortriptyline, and desipramine), tetracyclic antidepressants (e.g. mirtazapine) and monoamine oxidase inhibitors (e.g., phenelzine, isocarboxazid, and tranylcypromine), mebicar, fabomotizole, selank, bromantane, emoxypine, azapirones, barbiturates, hydroxyzine, pregabalin, validol, beta blockers (e.g., acebutolol, atenolol, betaxolol, bisoprolol, carteolol, carvedilol, Propofol, racetam-based drugs (e.g., aniracetam), alcohol, esmolol, labetalol, metoprolol, nadolol, nebivolol, penbutolol, pindolol, propranolol, sotalol, and timolol), and carbamates (e.g., meprobamate, tybamate, lorbamate).

In some embodiments, the active agent(s) is/are an antipsychotic(s). Exemplary antipsychotics include without limitation benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, clotiapine, loxapine, prothipendyl, clomipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, bifeprunox, bitopertin, brexpiprazole, cannabidiol, cariprazine, pimavanserin, pomaglumetad methionil, vabicaserin, xanomeline, and zicronapine.

In some embodiments, the active agent(s) is/are an analgesic(s). Exemplary analgesics include without limitation paracetamol/acetaminophen, non-steroidal anti-inflammants (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

In some embodiments, the active agent(s) is/are an antispasmodic(s). Exemplary antispasmodics include without limitation mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene.

In some embodiments, the active agent(s) is/are an antihistamine(s). Exemplary antihistamines include without limitation H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and β2-adrenergic agonists.

In some embodiments, the active agent(s) is/are an anti-infective(s). As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals. Exemplary anti-infectives include without limitation amebicides (e.g., nitazoxanide, paromomycin, metronidazole, tinidazole, chloroquine, and iodoquinol), aminoglycosides (e.g., paromomycin, tobramycin, gentamicin, amikacin, kanamycin, and neomycin), anthelmintics (e.g., benzimidazoles (e.g., albendazole, mebendazole, thiabendazole, fenbendazole, triclabendazole, flubendazole) abamectin, ivermectin, diethylcarbamazine, pyrantel pamoate, levamisole, silicylanilides (e.g., niclosamide, oxyclozanide), nitazoxanide, praziquantel, octadepsipeptides (e.g., emodepside), monepantel, spiroindoles (e.g., derquantel), artemisinin, moxidectin, milbemycins (e.g., milbemycin oxime), antifungals (e.g. azole antifungals (e.g., itraconazole, fluconazole, posaconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proguanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobici stat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, zidovudine, stavudine, emtricitabine, zalcitabine, telbivudine, simeprevir, boceprevir, telaprevir, lopinavir/ritonavir, fosamprenavir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, ceftaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, ceftizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, beta lactam antibiotics (benzathine penicillin (benzathine and benzylpenicillin), phenoxymethylpenicillin, cloxacillin, flucloxacillin, methicillin, temocillin, mecillinam, azlocillin, mezlocillin, piperacillin, amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, cefazolin, cephalexin, cephalosporin C, cephalothin, cefaclor, cefamandole, cefuroxime, cefotetan, cefoxitin, cefixime, cefotaxime, cefpodoxime, ceftazidime, ceftriaxone, cefepime, cefpirome, ceftaroline, biapenem, doripenem, ertapenem, faropenem, imipenem, meropenem, panipenem, razupenem, tebipenem, thienamycin, aztreonam, tigemonam, nocardicin A, taboxinine, and beta-lactam), quinolones (e.g., lomefloxacin, norfloxacin, ofloxacin, gatifloxacin, moxifloxacin, ciprofloxacin, levofloxacin, gemifloxacin, moxifloxacin, cinoxacin, nalidixic acid, enoxacin, grepafloxacin, trovafloxacin, and sparfloxacin), sulfonamides (e.g., sulfamethoxazole/trimethoprim, sulfasalazine, and sulfisoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, fosfomycin, cinoxacin, nalidixic acid, trimethoprim, and methylene blue).

In some embodiments, the active agent(s) is/are a chemotherapeutic(s). Exemplary chemotherapeutics include, without limitation, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, Bacillus Calmette-Guerin (BCG), temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In some embodiments, the gene modifying agent is an RNA guided nuclease or programmable nuclease, such as a CRISPR-Cas system, Meganuclease, Zinc Finger Nuclease and the like. Such systems are described in greater detail elsewhere herein.

### Scaffold Materials

In some embodiments, the culture condition can be the scaffold material in which the sample is associated with within the addressable array. As used herein, “scaffold material” and “scaffolds” refer to materials that can support cells and tissue and/or induce, stimulate, support, or otherwise contribute to one or more cellular interactions and/or cellular functions of cells associated with the scaffold material. The scaffold material can be biocompatible. The scaffold material can be natural or synthetic. The scaffold material can be porous. The scaffold can be formed of an array of microchannels in an otherwise solid substance. The scaffold can be a native scaffold (e.g., ECM, tissues, and the like). The scaffold material can be acellular. The scaffold material can be decellularized.

The scaffold material can be in any suitable three-dimensional form, including without limitation, gels, hydrogels, chips, membranes, sheets, morsels, putty, beads, particles (including macroparticles, microparticles, and nanofibers), fibers (including microfibers and nanofibers), nanowires, block structures, etc.

Exemplary scaffold materials that can be used in the addressable array include without limitation: bone, bone components and bone products (e.g., bone, partially mineralized bone, demineralized bone, hydroxyapatite, tri-calcium phosphate), hydrogels (e.g., graphene hydrogels, graphene-based hydrogels), cellulose (complete and decellularized), chitin/chitosan, alginate, agar, silk, cell extracellular matrix and components thereof (e.g., collagen, proteoglycans), hyaluronic acid, gelatin, polymers (e.g. PLA, PEG, PLGA, PGA, PMMA, PDLLA, PEE, PEO, PBT, PLLA, PLCL, poly(epsilon-caprolactone), PVA, PU, PEVA, polystyrene, polyesters), graphene, ceramics, peptides (e.g., self-assembling peptides), proteins (e.g., keratin), glass, and combinations thereof. See also, e.g., Campuzano et al., May 2019. Front. Sustain. Food Syst. https://doi.org/10.3389/fsufs.2019.00038; Carletti et al. Methods Mol Biol. 2011; 695:17-39; Dhaliwal, Anadika. 3d Cell Culture: A review. 2020. //dx.doi.org/10.13070/mm.en.2.162; Knight and Przyborski. 2014. J Anatomy. https://doi.org/10.1111/joa.12257; O'Brien, Fergal J. 2011. 14(3):88-95; Evans et al. Materials Today. 2006. 9(12): 26-33; Willerth and Sakiyama-Elbert. 2019. Stem J. 1(1)1-2, DOI: 10.3233/STJ-1800015; Edmondson et al., 2014. Assay Drug. Dev. Technol. 12(4):207-218; Lv et al. 2017. Oncology Lett. 14(6), 6999-7010 (https://doi.org/10.3892/ol.2017.7134); Betriu et al. J. Vis. Exp. 2018. 136, e57259, doi:10.3791/57259; and Castiaux et al., 2019. Analytical Method. 11:4220-4232).

### Cell Culture Type

In some embodiments, the culture condition can be cell culture type. As used herein “cell culture type” refers to the general methodology of how the cells and/or tissues are cultured. Exemplary and non-mutually exclusive cell culture types include, but are not limited to, two-dimensional, 3-dimensional, adherent, suspension, aggregate, spheroid, organoid, microfluidic, supercritical fluid-based, scaffold-based, and scaffold free.

As used herein, “microfluidic 3D cell culture” refers to a culture system in which cells are maintained in a predefined compartment by micropillars or other physical barriers, allowing interplay between perfused media.

As used herein, “hanging drop” in the context of cell culture, is a term of art that refers to culture that occurs in a drop of liquid that is suspended from a cover substrate that is placed over a cavity or depression in another substrate. This form of culture is often used for spheroid formation.

### Abiotic Stress

In some embodiments, the culture condition applied can be an abiotic stress. As used herein “abiotic stress” refers to non-living factors that can be applied to a sample that can or has the potential to impact or stress (positively or negatively) the sample. Non-living factors can be chemical and physical stresses. Exemplary abiotic stresses include without limitation temperature, light level, salinity, metals, tension and other forces applied to the sample, chemical active agents, non-living biologic active agents, partial pressure of different gasses present in the culture medium, radiation, magnetic fields, atmospheric pressure, and the like.

### Chemical Stress

In some embodiments, the culture condition applied can be a chemical stress. As used herein, “chemical stress” refers to non-living factors that are chemical compositions, compounds, or elements that can be applied to a sample that can or has the potential to impact or stress (positively or negatively) the sample. Exemplary chemical stressors include without limitation chemical active agents (described elsewhere herein), metals (e.g., heavy metals), salt concentration (salinity), gasses present in the culture medium, pH, small molecule pharmaceutical compounds, and the like.

### Biologic Stress

In some embodiments, the culture condition applied can be a biologic stress. As used herein, “biologic stress” refers to living organisms and viruses that can be applied to a sample that can or has the potential to impact or stress (positively or negatively) the sample. Biologic stress includes pathogenic and non-pathogenic organisms.

In some embodiments, the biologic stress is a fungi. Exemplary fungi include, without limitation, any type of fungi. In some embodiments, the fungi are within the phyla of Ascomycota, Basidiomycota, Blastocladiomycota, Chytridiomycota, Glomeromycota, Microsporidia, and Neocallimastigomycota. In some embodiments, the fungi can be Candida (e.g., C. albicans), Aspergillus (e.g., A. fumigatus, A. flavus, A. clavatus), Cryptococcus (e.g., C. neoformans, C. gattii), Histoplasma (H. capsulatum), Pneumocystis (e.g., P. jiroveecii), Stachybotrys (e.g., S. chartarum) and any combination thereof.

In some embodiments the biologic stress is a bacterium. Exemplary bacterium include any of those of the genus Actinomyces (e.g., A. israelii), Bacillus (e.g., B. anthracis, B. cereus), Bactereoides (e.g., B. fragilis), Bartonella (B. henselae, B. quintana), Bordetella (B. pertussis), Borrelia (e.g., B. burgdorferi, B. garinii, B. afzelii, and B. recurreentis), Brucella (e.g. B. abortus, B. canis, B. melitensis, and B. suis), Campylobacter (e.g., C. jejuni), Chlamydia (e.g., C. pneumoniae and C. trachomatis), Chlamydophila (e.g., C. psittaci), Clostridium (e.g., C. botulinum, C. difficile, C. perfringens. C. tetani), Corynebacterium (e.g., C. diptheriae), Enterococcus (e.g., E. Faecalis, E. faecium), Ehrlichia (E. canis and E. chaffensis) Escherichia (e.g., E. coli), Francisella (e.g., F. tularensis), Haemophilus (e.g., H. influenzae), Helicobacter (H. pylori), Klebsiella (e.g., K. pneumoniae), Legionella (e.g., L. pneumophila), Leptospira (e.g., L. interrogans, L. santarosai, L. weilii, L. noguchii), Listereia (e.g., L. monocytogeenes), Mycobacterium (e.g., M. leprae, M. tuberculosis, M. ulcerans), Mycoplasma (M. pneumoniae), Neisseria (e.g., N. gonorrhoeae and N. menigitidis), Nocardia (e.g., N. asteeroides), Pseudomonas (P. aeruginosa), Rickettsia (e.g., R. rickettsia), Salmonella (S. typhi and S. typhimurium), Shigella (e.g., S. sonnei and S. dysenteriae), Staphylococcus (e.g., S. aureus, S. epidermidis, and S. saprophyticus), Streeptococcus (e.g., S. agalactiaee, S. pneumoniae, S. pyogenes), Treponema (e.g., T. pallidum), Ureeaplasma (e.g., U. urealyticum), Vibrio (e.g., V. cholerae), Yersinia (e.g., Y. pestis, Y. enteerocolitica, and Y. pseudotuberculosis), and/or combinations thereof.

In some embodiments, the biotic stress is a parasite. Exemplary parasites include without limitation, Acanthamoeba spp., Balamuthia mandrillaris, Babesiosis spp. (e.g., Babesia B. divergens, B. bigemina, B. equi, B. microfti, B. duncani), Balantidiasis spp. (e.g., Balantidium coli), Blastocystis spp., Cryptosporidium spp., Cyclosporiasis spp. (e.g., Cyclospora cayetanensis), Dientamoebiasis spp. (e.g., Dientamoeba fragilis), Amoebiasis spp. (e.g., Entamoeba histolytica), Giardiasis spp. (e.g., Giardia lamblia), Isosporiasis spp. (e.g., Isospora belli), Leishmania spp., Naegleria spp. (e.g., Naegleria fowleri), Plasmodium spp. (e.g., Plasmodium falciparum, Plasmodium vivax, Plasmodium ovale curtisi, Plasmodium ovale wallikeri, Plasmodium malariae, Plasmodium knowlesi), Rhinosporidiosis spp. (e.g., Rhinosporidium seeberi), Sarcocystosis spp. (e.g., Sarcocystis bovihominis, Sarcocystis suihominis), Toxoplasma spp. (e.g., Toxoplasma gondii), Trichomonas spp. (e.g., Trichomonas vaginalis), Trypanosoma spp. (e.g., Trypanosoma brucei), Trypanosoma spp. (e.g., Trypanosoma cruzi), Tapeworm (e.g., Cestoda, Taenia multiceps, Taenia saginata, Taenia solium), Diphyllobothrium latum spp., Echinococcus spp. (e.g., Echinococcus granulosus, Echinococcus multilocularis, E. vogeli, E. oligarthrus), Hymenolepis spp. (e.g., Hymenolepis nana, Hymenolepis diminuta), Bertiella spp. (e.g., Bertiella mucronata, Bertiella studeri), Spirometra (e.g. Spirometra erinaceieuropaei), Clonorchis spp. (e.g., Clonorchis sinensis; Clonorchis viverrini), Dicrocoelium spp. (e.g., Dicrocoelium dendriticum), Fasciola spp. (e.g., Fasciola hepatica, Fasciola gigantica), Fasciolopsis spp. (e.g., Fasciolopsis buski), Metagonimus spp. (e.g., Metagonimus yokogawai), Metorchis spp. (e.g., Metorchis conjunctus), Opisthorchis spp. (e.g., Opisthorchis viverrini, Opisthorchis felineus), Clonorchis spp. (e.g., Clonorchis sinensis), Paragonimus spp. (e.g., Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g., Schistosoma mansoni, Schistosoma haematobium, Schistosoma japonicum, Schistosoma mekongi, and Schistosoma intercalatum), Echinostoma spp. (e.g., E. echinatum), Trichobilharzia spp. (e.g., Trichobilharzia regent), Ancylostoma spp. (e.g., Ancylostoma duodenale), Necator spp. (e.g., Necator americanus), Angiostrongylus spp., Anisakis spp., Ascaris spp. (e.g., Ascaris lumbricoides), Baylisascaris spp. (e.g., Baylisascaris procyonis), Brugia spp. (e.g. Brugia malayi, Brugia timori), Dioctophyme spp. (e.g., Dioctophyme renale), Dracunculus spp. (e.g., Dracunculus medinensis), Enterobius spp. (e.g., Enterobius vermicularis, Enterobius gregorii), Gnathostoma spp. (e.g., Gnathostoma spinigerum, Gnathostoma hispidum), Halicephalobus spp. (e.g., Halicephalobus gingivalis), Loa loa spp. (e.g., Loa loa filaria), Mansonella spp. (e.g., Mansonella streptocerca), Onchocerca spp. (e.g., Onchocerca volvulus), Strongyloides spp. (e.g., Strongyloides stercoralis), Thelazia spp. (e.g., Thelazia californiensis, Thelazia callipaeda), Toxocara spp. (e.g., Toxocara canis, Toxocara cati, Toxascaris leonine), Trichinella spp. (e.g., Trichinella spiralis, Trichinella britovi, Trichinella nelsoni, Trichinella nativa), Trichuris spp. (e.g., Trichuris trichiura, Trichuris vulpis), Wuchereria spp. (e.g., Wuchereria bancrofti), Dermatobia spp. (e.g., Dermatobia hominis), Tunga spp. (e.g., Tunga penetrans), Cochliomyia spp. (e.g., Cochliomyia hominivorax), Linguatula spp. (e.g., Linguatula serrata), Archiacanthocephala sp., Moniliformis sp. (e.g. Moniliformis moniliformis), Pediculus spp. (e.g., Pediculus humanus capitis, Pediculus humanus humanus), Pthirus spp. (e.g., Pthirus pubis), Arachnida spp. (e.g., Trombiculidae, Ixodidae, Argaside), Siphonaptera spp (e.g., Siphonaptera: Pulicinae), Cimicidae spp. (e.g., Cimex lectularius and Cimex hemipterus), Diptera spp., Demodex spp. (e.g., Demodex folliculorum/brevis/canis), Sarcoptes spp. (e.g., Sarcoptes scabiei), Dermanyssus spp. (e.g., Dermanyssus gallinae), Ornithonyssus spp. (e.g., Ornithonyssus sylviarum, Ornithonyssus bursa, Ornithonyssus bacoti), Laelaps spp. (e.g., Laelaps echidnina), Liponyssoides spp. (e.g., Liponyssoides sanguineus), and/or combinations thereof.

In some embodiments, the biotic stress can be a virus. Exemplary viruses include without limitation a double-stranded DNA virus, a partly double-stranded DNA virus, a single-stranded DNA virus, a positive single-stranded RNA virus, a negative single-stranded RNA virus, or a double stranded RNA virus. In some embodiments, the pathogenic virus can be from the family Adenoviridae (e.g., Adenovirus), Herpesviridae (e.g., Herpes simplex, type 1, Herpes simplex, type 2, Varicella-zoster virus, Epstein-Barr virus, Human cytomegalovirus, Human herpesvirus, type 8), Papillomaviridae (e.g., Human papillomavirus), Polyomaviridae (e.g., BK virus, JC virus), Poxviridae (e.g., smallpox), Hepadnaviridae (e.g. Hepatitis B), Parvoviridae (e.g., Parvovirus B19), Astroviridae (e.g. Human astrovirus), Caliciviridae (e.g., Norwalk virus), Picornaviridae (e.g., coxsackievirus, hepatitis A virus, poliovirus, rhinovirus), Coronaviridae (e.g., Severe acute respiratory syndrome-related coronavirus, strains: Severe acute respiratory syndrome virus, Severe acute respiratory syndrome coronavirus 2 (COVID-19)), Flaviviridae (e.g., Hepatitis C virus, yellow fever virus, dengue virus, West Nile virus, TBE virus), Togaviridae (e.g., Rubella virus), Hepeviridae (e.g., Hepatitis E virus), Retroviridae (Human immunodeficiency virus (HIV)), Orthomyxoviridae (e.g., Influenza virus), Arenaviridae (e.g., Lassa virus), Bunyaviridae (e.g., Crimean-Congo hemorrhagic fever virus, Hantaan virus), Filoviridae (e.g. Ebola virus and Marburg virus), Paramyxoviridae (e.g., Measles virus, Mumps virus, Parainfluenza virus, Respiratory syncytial virus), Rhabdoviridae (e.g., Rabies virus), Hepatitis D virus, Reoviridae (e.g., Rotavirus, Orbivirus, Coltivirus, Banna virus), and combinations thereof.

### Physical Stress

In some embodiments, the culture conditions applied is a physical stress. As used herein, “physical stress” refers to non-living factors that relate to physical properties that can be applied to a sample that can or has the potential to impact or stress (positively or negatively) the sample. Exemplary physical stressors include without limitation atmospheric pressures above and below the pressure at sea level, temperature, electromagnetic force, current applied to the sample light (intensity and/or wavelength), axial tensions applied to the sample, axial compression to the sample, torsional tension applied to the sample, torsional compression applied to the sample, bending force applied to the sample, gravitational force, frictional force, centripetal force, and combinations thereof.

### CRISPR-Cas and Other Nucleic Acid Targeting Systems

In some embodiments, the combinatorial addressable array or array feature can be or include a CRISPR-Cas system or component thereof.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

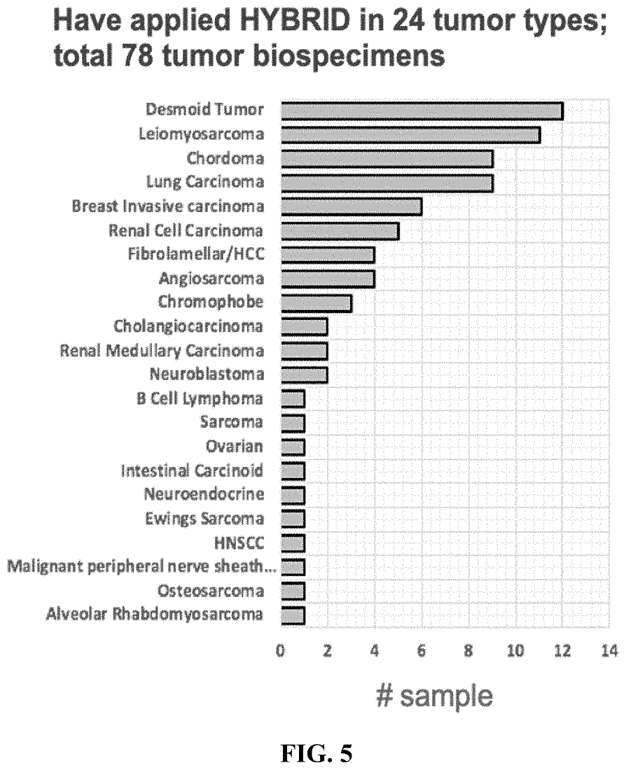

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83, particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system includes 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cash 1). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cash, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SETT/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Application Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstituble halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system. See e.g., Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

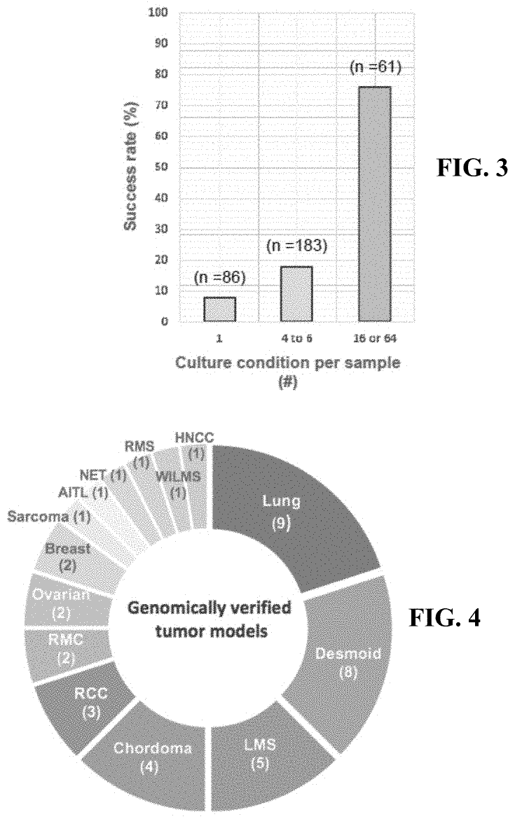

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, and 4.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 1 below shows several Cas polypeptides and the PAM sequence they recognize.

TABLE 1
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′ end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the MARC polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. See e.g., Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160. Increased cleavage specificity can be attained with decreased off target activity by use of paired ZFN heterodimers, each targeting different nucleotide sequences separated by a short spacer. See e.g., Doyon, Y. et al., 2011, Enhancing zinc-finger-nuclease activity with improved obligate heterodimeric architectures. Nat. Methods 8, 74-79. ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a MARC polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35), where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 1)
MDPIRSRTPSPARELLSGPQPDGVQPTADRGVSPPAGGPLDGLP
ARRTMSRTRLPSPPAPSPAFSADSFSDLLRQFDPSLFNTSLFDS
LPPFGAHHTEAATGEWDEVQSGLRAADAPPPTMRVAVTAARP
PRAKPAPRRRAAQPSDASPAAQVDLRTLGYSQQQQEKIKPKVR
STVAQHHEALVGHGFTHAHIVALSQHPAALGTVAVKYQDMIA
ALPEATHEAIVGVGKQWSGARALEALLTVAGELRGPPLQLDT
GQLLKIAKRGGVTAVEAVHAWRNALTGAPLN

An exemplary amino acid sequence of a C-terminal capping region is:

(SEQ ID NO: 2)
RPALESIVAQLSRPDPALAALTNDHLVALACLGGRPALDAVKK
GLPHAPALIKRTNRRIPERTSHRVADHAQVVRVLGFFQCHSHP
AQAFDDAMTQFGMSRHGLLQLFRRVGVTELEARSGTLPPASQ
RWDRILQASGMKRAKPSPTSTQTPDQASLHAFADSLERDLDAP
SPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a MARC polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### Sequences Related to Nucleus Targeting and Transportation

In some embodiments, one or more components of the CRISPR-Cas or other nucleic acid targeting system or a component thereof (e.g., the Cas protein and/or deaminase) can include one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 3) or PKKKRKVEAS (SEQ ID NO: 4); the NLS from nucleoplasmin (e.g., the nucleoplasmin bipartite NLS with the sequence KRPAATKKAGQAKKKK (SEQ ID NO: 5)); the c-myc NLS having the amino acid sequence PAAKRVKLD (SEQ ID NO: 6) or RQRRNELKRSP (SEQ ID NO: 7); the hRNPA1 M9 NLS having the sequence NQSSNFGPMKGGNFGGRSSGPYGGGGQYFAKPRNQGGY (SEQ ID NO: 8); the sequence RMRIZFKNKGKDTAELRRRRVEVSVELRKAKKDEQILKRRNV (SEQ ID NO: 9) of the IBB domain from importin-alpha; the sequences VSRKRPRP (SEQ ID NO: 10) and PPKKARED (SEQ ID NO: 11) of the myoma T protein; the sequence PQPKKKPL (SEQ ID NO: 12) of human p53; the sequence SALIKKKKKMAP (SEQ ID NO: 13) of mouse c-abl IV; the sequences DRLRR (SEQ ID NO: 14) and PKQKKRK (SEQ ID NO: 15) of the influenza virus NS1; the sequence RKLKKKIKKL (SEQ ID NO: 16) of the Hepatitis virus delta antigen; the sequence REKKKFLKRR (SEQ ID NO: 17) of the mouse Mx1 protein; the sequence KRKGDEVDGVDEVAKKKSKK (SEQ ID NO: 18) of the human poly(ADP-ribose) polymerase; and the sequence RKCLQAGMNLEARKTKK (SEQ ID NO: 19) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g., aptamers) for adapter proteins, which may be linked to or fused to a nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g., due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. In some embodiments, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the CRISPR-Cas system or other nucleic acid targeting system can include a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Samples

In some embodiments, one or more of the discrete locations of the plurality of discrete locations on the addressable array comprises a sample. The sample can be cell(s), tissue, spheroid, organoid, or a combination thereof. In some embodiments, one or more of the discrete locations of the plurality of discrete locations on the addressable array comprises non-sample cell(s), tissue, spheroid, organoid, or a combination thereof. The sample and/or or non-sample cells, tissue, and/or organoid are cancer cells, cancer tissue, or cancer organoid or are generated from one or more cancer cells. In some embodiments, sample and/or non-sample can be or contain a homogenous cell population. In some embodiments, sample and/or non-sample can be or contain a heterogenous cell population.

The samples that can be any cell, cell population, tissue, extracellular component, bodily fluid, bodily excretion, bodily secretion, or any combinations thereof. The sample can be from diseased tissue or cells. The sample can be from a solid tumor. The sample can be any cell or tissue type. The sample can be of limited nature, meaning that only a small amount of sample is available for analysis. As discussed elsewhere herein, the combinatorial array and methods described herein are capable of maximizing the amount of information about a sample and therefore are particularly useful for samples where a limited amount is available.

The sample can be obtained from a subject by any suitable method. In some embodiments the sample is obtained by a biopsy needle.

In some embodiments, the amount of sample that is available for use in the combinatorial array ranges from about 1 to about 100 ng, mg, or g or more. In some embodiments, the amount of sample that is available for use in the combinatorial array can be less than about 100, less than about 10, or less than about 1 mg, or ng.

### Array Fabrication

Arrays can be fabricated using any suitable fabrication method(s) or technique(s), including but not limited to additive manufacturing and other 3D printing techniques, etching, engraving, molding (e.g., injection, rotational, blow etc.), casting (e.g., centrifugal casting, continuous casting, die casting, evaporative-pattern casting, and investment casting), forming (e.g., end tube forming, forging, rolling, extrusion, pressing, and bending), joining (e.g., welding, brazing, soldering, sintering, bonding, fastening and press fitting), machining (e.g., milling, turning, drilling, reaming, countersinking, tapping, sawing, broaching, and shaping), labeling and painting (e.g., engraving, ink jet printing, chemical vapor deposition, etc.). In some embodiments, array fabrication can include using drop deposition of features. Such methods are described in detail, for example, in U.S. Pat. Nos. 6,242,266, 6,232,072, 6,180,351, 6,171,797, and 6,323,043, which are incorporated herein by reference.

### Array Systems

The combinatorial addressable arrays described herein can be part of a system that can facilitate use and/or automation of one or more aspects of the combinatorial addressable arrays described herein.

In some embodiments, the system can include one or more machines or devices configured to form one or more of the array features. Such machines or devices can be operatively coupled, fluidically coupled, or otherwise coupled to the combinatorial addressable array.

In some embodiments, the system can include one or more machines or devices configured to deliver and/or remove one or more reagents to one or more positions of the combinatorial addressable array. Such machines or devices can be operatively coupled, fluidically coupled, or otherwise coupled to the combinatorial addressable array.

In some embodiments, the system can include one or more machines, devices, or other component that is/are configured to maintain or modulate an array environment. Such machines, devices, and components, include but are not limited to, gas reservoirs, heaters, coolers, humidifiers, dehumidifiers, pressurizers, lights, shades (to create a dark environment), and combinations thereof.

In some embodiments, the system can include one or more machines or devices that are configured to deliver and/or remove a sample and/or sample progeny from the combinatorial addressable array. Such machines or devices can be operatively coupled, fluidically coupled, or otherwise coupled to the combinatorial addressable array.

In some embodiments, the system can include on one or more machines or devices that are configured to detect or measure a characteristic, product, feature, or other aspect of a sample or sample progeny present in the combinatorial addressable array. Such machines or devices can be operatively coupled, optically coupled, electrically coupled, magnetically coupled, biologically coupled, fluidically coupled, or otherwise coupled to the combinatorial addressable array. Exemplary devices include, but are not limited to sequencers, thermocyclers, microscopes, light sources, cell sorters (e.g., FACS sorters and microfluidic cell sorters), spectrometers, HPLCs, mass spectrometers, gas chromatographs, scintillators, and other live-cell analyzers (e.g., cytometers, imagers, metabolic analyzers).

In some embodiments, the system can include one or more machines or devices that are configured to receive, process, and analyze an output from the one or more machines or devices that are configured to detect or measure a characteristic, product, feature, or other aspect of a sample or sample progeny present in the combinatorial addressable array. Such devices can also be configured to provide an output to a user in a suitable format.

In some embodiments, the system can include one or more machines or devices that are configure to clean the one or more components of the system and/or combinatorial addressable array. Such machines or devices can be operatively coupled, optically coupled, electrically coupled, magnetically coupled, fluidically coupled, or otherwise coupled to the combinatorial addressable array.

### Methods of High-Throughput Empirical Determination of Optimal Culture Conditions

Also described herein are embodiments of high-throughput methods of empirically determining culture conditions effective to modify a biological sample that can include culturing a biological sample having an initial characteristic state in one or more of the discrete locations on the combinatorial addressable array described herein; and determining a change in the initial state of a characteristic of the biological sample, wherein the change in the initial state of the characteristic identifies one or more conditions effective to modify the characteristic in the biological sample.

In some embodiments determining a change in the characteristic of the biological sample comprises performing gene and/or genome sequencing, a gene expression analysis, an epigenetic analysis, a cell phenotype analysis, a cell morphology analysis, a growth analysis, a differentiation analysis, a cell volume analysis, a cell viability analysis, a cell metabolism analysis, a cell communication or signal transduction analysis, a cell reproduction analysis, a cell response analysis, a cell production or secretion analysis, a cell function analysis or a combination thereof.

In some embodiments, the step of determining a change in the characteristic of the sample can include generating a signature.

As used herein, the term “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. A signature can be composed of any number of genes, proteins epigenetic elements, and/or combinations thereof. For example, a gene signature may include a list of genes differentially expressed in a distinction of interest. The signature can be composed completely of or contain 1-1,000 or more genes, proteins or elements, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 or more genes, proteins and/or epigenetic elements, including any range of values therein (e.g. 1-10, 2-10, 5-273, etc.). In some embodiments, the signature can be composed completely of or contain 1-20 or more, 2-20 or more, 3-20 or more, 4-20 or more, 5-20 or more, 6-20 or more, 7-20 or more, 8-20 or more, 9-20 or more, 10-20 or more, 11-20 or more, 12-20 or more, 13-20 or more, 14-20 or more, 15-20 or more, 16-20 or more, 17-20 or more, 18-20 or more, 19-20 or more, or 20 or more genes, proteins and/or epigenetic elements.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signatures of the present invention may be discovered by analysis of expression profiles of single-cells within a population of cells from isolated samples (e.g., blood samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cancer cells that are linked to particular pathological condition (e.g., cancer grade), or linked to a particular outcome or progression of the disease or linked to a particular response to treatment of the disease.

In certain embodiments, a signature is characterized as being specific for a particular tumor cell or tumor cell (sub)population if it is upregulated or only present, detected or detectable in that particular tumor cell or tumor cell (sub)population, or alternatively is downregulated or only absent, or undetectable in that particular tumor cell or tumor cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

As discussed herein, differentially expressed genes/proteins, or differential epigenetic elements may be differentially expressed on a single cell level or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of tumor cells. As referred to herein, a “subpopulation” of cells preferably refers to a particular subset of cells of a particular cell type which can be distinguished or are uniquely identifiable and set apart from other cells of this cell type. The cell subpopulation may be phenotypically characterized and is preferably characterized by the signature as discussed herein. A cell (sub)population as referred to herein may constitute of a (sub)population of cells of a particular cell type characterized by a specific cell state.

Signatures may be functionally validated as being uniquely associated with a particular immune responder phenotype. Induction or suppression of a particular signature may consequentially be associated with or causally drive a particular immune responder phenotype.

Various aspects and embodiments of the invention may involve analyzing gene signatures, protein signature, and/or other genetic or epigenetic signature based on single cell analyses (e.g., single cell RNA sequencing) or alternatively based on cell population analyses, as is defined herein elsewhere.

In some embodiments, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

One or more biomarkers (genes, proteins, epigenetic features etc.) can be detected and may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g., diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

In some embodiments, the signature genes, biomarkers, and/or cells may be detected using an immunoassay. Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see e.g., ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Other formats can be used in connection with the combinatorial addressable arrays described herein. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

In some embodiments, the signature genes, biomarkers, and/or cells may be detected using a hybridization assay. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization with Nucleic Acid Probes”, Elsevier Science Publishers B.V. (1993) and Kricka, “Nonisotopic DNA Probe Techniques”, Academic Press, San Diego, Calif. (1992).

In some embodiments, the signature genes, biomarkers, and/or cells may be detected using a sequencing technique. Suitable sequencing techniques include but are not limited to, single molecule. Real time (SMART) sequencing (see e.g., Eid et al., 2009, Science. 323(5910):133-138), nanopore sequencing (see e.g., Bowden et al., 2019, Nature Comm. 10(1869), https://doi.org/10.1038/s41467-019-09637-5), massively parallel signature sequencing (MPSS) (see e.g., Brenner et al., 2000. Nature Biotech. 18(6):630-634) polony sequencing (see e.g., Shendure et al., 2009, Science. 309(5741):1728-1732), 454 pyrosequencing (see e.g., Margulies et al. Nature. 437 (7057):376-380), Solexa/Illumina sequencing (see e.g., Bentley et al. Nature. 456(72318):53-59, Mardis et al., 2008. Annu rev Genom Hum Genet. 9:387-402), combinatorial probe anchor synthesis (cPAS) sequencing (see e.g., Drmanac et al. 2010, Science. 327 (5961): 78-81), SOLiD sequencing (see e.g., Valouev et al., 2008. Genome Res. 18(7): 1051-1063), Ion torrent semiconductor sequencing (see e.g., Rusk et al., 2011. Nature Methods. 8(1):44), DNA nanoball sequencing (see e.g., Porreca G J. 2010. Nature Biotech. 28(1):43-44), heliscope single molecule sequencing (see e.g., Thompson et al., 2010. doi:10.1002/0471142727.mb0710s92), microfluidic system-based sequencing (including but not limited to droplet based microfluidic sequencing methods and digital microfluidic sequencing methods (see e.g., Abate et al., 2013. Lab on a Chip 13(24) doi:10.1039/c31c50905b, Pekin et al., 2011. Lab on a Chip 11(13):2156-2166; Fair et al., 2007. IEE Design & Test of Computers. 24(1):10-24, Boles et al., 2011. Anal. Chem. 83(22):8439-8447, Zilionis et al., 2017, Nature Protocols. 12(1):44-73, Kan et al., 2004. Electrophoresis. 25 (21-22): 3564-3588, and others described elsewhere herein), tunneling currents sequencing (see e.g., Di Ventra. 2013. Nanotechnology. 24(34):342501, sequencing by hybridization (see e.g., Hanna et al., 2000. J. Clin. Microbiol. 38(7):2715-2721, Morey et al., 2013. Molecular Genetics and Metabolism. 110(1-2):3-24), sequencing with mass spectrometry (see e.g., Edwards et al., 2005. Mutation Research. 573(1-2):3-12, Hall et al. 2005. Analytical Biochem. 344(1): 53-69, Monforte et al. 1997. Nature Medicine 3(3):360-362, Beres et al. PNAS. 107(9):4371-4376), microscopy-based sequencing techniques (see e.g., Bell et al., 2012. Microscopy and Microanalysis) 18 (5):1049-1053), and RNA polymerase-based sequencing (RNAP) and combinations thereof.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (See e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

In some embodiments, the expression of one or more genes, proteins, epigenetic features and the like can deviate from first measurement (such as in an initial cell state) and a second measurement taken after a period of time and/or under different culture condition(s).

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or 90% or 95% or even 00% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar. In some embodiments a machine learning or other statistical model, such as any of those described elsewhere herein can be used.

In some embodiments the method involves disrupting a sample present on the addressable array. In some embodiments them method does not disrupt the sample present on the addressable array. In some embodiments, the step of determining a change can occur multiple times (e.g., 2, 3, 4, 5, 6, 7, 8, 9, 10, 11, 12, 13, 14, 15 times or more) over a period of time after introducing the sample to the combinatorial addressable array. In some embodiments, the step of determining a change can occur after altering the culture conditions.

The characteristic measured can be any suitable and/or desired characteristic. Exemplary characteristics include, but are not limited to, the characteristic is growth, differentiation, proliferation, organoid formation, viability, death/apoptosis, cell product production and/or secretion, gene expression, protein expression, epigenome state, metabolism, cell volume, cell size, cell state, cell type or subtype, cell morphology, or a combination thereof.

### Trained Models to Optimize Cell Culture Conditions

Described herein are trained models, such as machine learning models, that can be used in conjunction with the combinatorial arrays described elsewhere herein to determine an optimal cell culture conditions. In some embodiments, the use of the trained model to optimize culture conditions in connection with the combinatorial arrays described herein can lead to a determination of particular cell culture conditions for a sample that could not previously be realized. This can be the case where the sample amount available is limited such that conventional techniques and methods cannot perform a sufficient amount or type of the targeted iterations of culture conditions so as to arrive at an optimal cell culture condition to further propagate the sample cells for additional analysis and diagnostics on the sample and/or sample progeny. Indeed, this has been a gate in developing personalized therapies for many diseases and cancers simply because there is not a sufficient amount of sample so as to allow for optimization of culture conditions so the in vitro assays and manipulations can be performed.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ, SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FP-growth algorithm; Hierarchical clustering, such as Single-linkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory.

In some embodiments, described herein are computer-implemented methods of training a statistical or machine learning model for determining and/or predicting culture conditions effective for growth of a biologic sample, comprising: collecting a set of sample culture parameters from a database to generate a collected set of sample culture parameters; applying one or more transformations to each sample culture parameters to create a modified set of sample culture parameters; creating a first training set comprising the collected set of sample culture parameters, the modified set of sample culture parameters, and a set of non-effective sample culture parameter results; training a statistical model or machine learning algorithm in a first stage using the first training set; optionally creating a second training set for a second stage of training comprising the first training set and optionally, sample culture parameters that are incorrectly detected as effective sample culture parameters after the first stage of training; and optionally training the neural network in a second stage using the second training set.

In some embodiments, the database comprises one or more of the following: one or more clinical annotations of biologic samples, treatment response history of biologic samples, cell culture condition response and/or optimal parameters of/for biologic samples, processing method history of biologic samples, phenotype of biologic samples, genomic profile of biologic samples, epigenomic profile of biologic samples, and biologic sample source annotations. In some embodiments, the one or more clinical annotations can be any one or more of those set forth in Appendix A, U.S. Provisional Application Ser. No. 63/057,812, which is incorporated by reference as if expressed in its entirety herein.

In some embodiments, the computer-implemented the statistical model or machine learning algorithm is configured as a neural network, decision tree, support vector machine, linear regression, logistical regression, random forest, gradient boosted trees, naive bayes, nearest neighbor, k-means clustering, t-SNE, principal component analysis, association rule, Q-learning, temporal difference, Monte-Carlo tree search, asynchronous actor-critic agents, or any permissible combination thereof. Other suitable configurations will be appreciated in view of the description herein.

### Methods of Using the Trained Models.

The trained models described herein can be used in a method to determine and/or predict culture conditions effective for growth of a biologic sample, such as a sample present in the combinatorial addressable array. In some embodiments, the computer-implemented method for determining and/or predicting culture conditions effective for growth of a biologic sample, comprises: receiving biologic sample data; optionally applying one or more filters to the biologic sample data; using the received biologic sample data or filtered biologic sample data as input and applying a one or more classifiers to determine and/or predict one or more effective biologic sample biologic sample culture conditions based on a computer-accessible database, trained statistical or machine-learning model trained to predict effective biologic sample culture conditions based on the one or more classifiers, a statistical data analysis methodology, or a combination thereof.

In some embodiments, the one or more determined and/or predicted effective biologic sample culture conditions are passed through one or more additional filters to further optimize the determined and/or predicted effective biologic sample culture conditions.

In some embodiments, the computer-implemented method can further comprise applying one or more additional classifiers to the one or more determined and/or predicted effective biologic sample culture conditions or further optimized determined and/or predicted effective culture conditions to determine and/or predict one or more effective biologic sample biologic sample culture conditions based on the computer-accessible database and/or trained machine-learning model trained to predict effective biologic sample culture conditions based on the one or more additional classifiers.

In some embodiments the trained statistical or machine-learning model is produced by a method as described elsewhere herein (see e.g., “Trained Models to Optimize Cell Culture Conditions”). In some embodiments, the biologic sample data is received from user input, one or more sensors, one or more detection devices, one or more sample characteristic measurement and/or analysis devices, a database, or a combination thereof.

In some embodiments, the biological sample is contained in a combinatorial addressable array as described elsewhere herein.

In some embodiments, described herein are computer-implemented methods to determine and/or predict culture conditions effective growth for growth of a biologic sample, comprising: receiving data of one or more parameters from the biologic sample in a format usable by a computing device; executing processing logic configured to generate feature data from the received data, filter the received data and/or the feature data, and/or process the feature data and/or received data with one or more trained machine learning models that is/are trained to predict effective biologic sample culture conditions based on the received data and/or feature data; and executing processing logic configured to cause a list of the effective biologic sample culture conditions to be displayed via an electronic display, transmitted to a user interface program, and/or be saved to a non-transitory computer readable memory.

In some embodiments, at least one of the one or more trained statistical or machine learning models are produced by a method as described elsewhere herein (see e.g., “Trained Models to Optimize Cell Culture Conditions”).

In some embodiments, the data of one or more parameters is received from user input, one or more sensors, one or more detection devices, one or more sample characteristic measurement and/or analysis devices, a database, or a combination thereof.

In some embodiments, the biological sample is contained in a combinatorial addressable array as described elsewhere herein.

### Devices and Systems for Determining Optimal Cell Culture Conditions Using the Trained Models.

Described herein are devices and systems that can be used to determine optimal cell culture conditions using the trained models. Such systems can also, in some embodiments, be capable of developing and/or training the trained models described elsewhere herein.

In some embodiments, a non-transitory computer readable medium comprises computer-executable instructions recorded thereon for causing a computer to perform a computer-implemented method described elsewhere herein.

In some embodiments, a system can comprise non-transitory computer-readable medium; and a processor configured to execute instructions stored on the non-transitory computer readable medium which, when executed, cause the processor to perform a computer-implemented method described elsewhere herein.

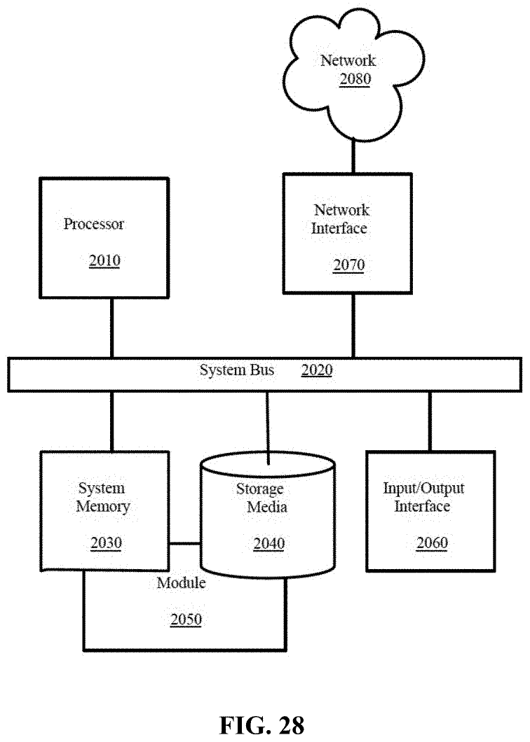

FIG. 28 depicts a computing machine 2000 and a module 2050 in accordance with certain example embodiments. The computing machine 2000 may correspond to any of the various computers, servers, mobile devices, embedded systems, or computing systems presented herein. The module 2050 may comprise one or more hardware or software elements configured to facilitate the computing machine 2000 in performing the various methods and processing functions presented herein. The computing machine 2000 may include various internal or attached components such as a processor 2010, system bus 2020, system memory 2030, storage media 2040, input/output interface 2060, and a network interface 2070 for communicating with a network 2080.

The computing machine 2000 may be implemented as a conventional computer system, an embedded controller, a laptop, a server, a mobile device, a smartphone, a set-top box, a kiosk, a vehicular information system, one more processors associated with a television, a customized machine, any other hardware platform, or any combination or multiplicity thereof. The computing machine 2000 may be a distributed system configured to function using multiple computing machines interconnected via a data network or bus system.

The processor 2010 may be configured to execute code or instructions to perform the operations and functionality described herein, manage request flow and address mappings, and to perform calculations and generate commands. The processor 2010 may be configured to monitor and control the operation of the components in the computing machine 2000. The processor 2010 may be a general purpose processor, a processor core, a multiprocessor, a reconfigurable processor, a microcontroller, a digital signal processor (“DSP”), an application specific integrated circuit (“ASIC”), a graphics processing unit (“GPU”), a field programmable gate array (“FPGA”), a programmable logic device (“PLD”), a controller, a state machine, gated logic, discrete hardware components, any other processing unit, or any combination or multiplicity thereof. The processor 2010 may be a single processing unit, multiple processing units, a single processing core, multiple processing cores, special purpose processing cores, coprocessors, or any combination thereof. According to certain embodiments, the processor 2010 along with other components of the computing machine 2000 may be a virtualized computing machine executing within one or more other computing machines.

The system memory 2030 may include non-volatile memories such as read-only memory (“ROM”), programmable read-only memory (“PROM”), erasable programmable read-only memory (“EPROM”), flash memory, or any other device capable of storing program instructions or data with or without applied power. The system memory 2030 may also include volatile memories such as random access memory (“RAM”), static random access memory (“SRAM”), dynamic random access memory (“DRAM”), and synchronous dynamic random access memory (“SDRAM”). Other types of RAM also may be used to implement the system memory 2030. The system memory 2030 may be implemented using a single memory module or multiple memory modules. While the system memory 2030 is depicted as being part of the computing machine 2000, one skilled in the art will recognize that the system memory 2030 may be separate from the computing machine 2000 without departing from the scope of the subject technology. It should also be appreciated that the system memory 2030 may include, or operate in conjunction with, a non-volatile storage device such as the storage media 2040.

The storage media 2040 may include a hard disk, a floppy disk, a compact disc read only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 2040 may store one or more operating systems, application programs and program modules such as module 2050, data, or any other information. The storage media 2040 may be part of, or connected to, the computing machine 2000. The storage media 2040 may also be part of one or more other computing machines that are in communication with the computing machine 2000 such as servers, database servers, cloud storage, network attached storage, and so forth.

The module 2050 may comprise one or more hardware or software elements configured to facilitate the computing machine 2000 with performing the various methods and processing functions presented herein. The module 2050 may include one or more sequences of instructions stored as software or firmware in association with the system memory 2030, the storage media 2040, or both. The storage media 2040 may therefore represent examples of machine or computer readable media on which instructions or code may be stored for execution by the processor 2010. Machine or computer readable media may generally refer to any medium or media used to provide instructions to the processor 2010. Such machine or computer readable media associated with the module 2050 may comprise a computer software product. It should be appreciated that a computer software product comprising the module 2050 may also be associated with one or more processes or methods for delivering the module 2050 to the computing machine 2000 via the network 2080, any signal-bearing medium, or any other communication or delivery technology. The module 2050 may also comprise hardware circuits or information for configuring hardware circuits such as microcode or configuration information for an FPGA or other PLD. [0131] The input/output (“I/O”) interface 2060 may be configured to couple to one or more external devices, to receive data from the one or more external devices, and to send data to the one or more external devices. Such external devices along with the various internal devices may also be known as peripheral devices. The I/O interface 2060 may include both electrical and physical connections for operably coupling the various peripheral devices to the computing machine 2000 or the processor 2010. The I/O interface 2060 may be configured to communicate data, addresses, and control signals between the peripheral devices, the computing machine 2000, or the processor 2010. The I/O interface 2060 may be configured to implement any standard interface, such as small computer system interface (“SCSI”), serial-attached SCSI (“SAS”), fiber channel, peripheral component interconnect (“PCP”), PCI express (PCIe), serial bus, parallel bus, advanced technology attached (“ATA”), serial ATA (“SAT A”), universal serial bus (“USB”), Thunderbolt, Fire Wire, various video buses, and the like. The I/O interface 2060 may be configured to implement only one interface or bus technology. Alternatively, the I/O interface 2060 may be configured to implement multiple interfaces or bus technologies. The I/O interface 2060 may be configured as part of, all of, or to operate in conjunction with, the system bus 2020. The I/O interface 2060 may include one or more buffers for buffering transmissions between one or more external devices, internal devices, the computing machine 2000, or the processor 2010.

The I/O interface 2060 may couple the computing machine 2000 to various input devices including mice, touch-screens, scanners, biometric readers, electronic digitizers, sensors, receivers, touchpads, trackballs, cameras, microphones, keyboards, any other pointing devices, or any combinations thereof. The I/O interface 2060 may couple the computing machine 2000 to various output devices including video displays, speakers, printers, projectors, tactile feedback devices, automation control, robotic components, actuators, motors, fans, solenoids, valves, pumps, transmitters, signal emitters, lights, and so forth.

The computing machine 2000 may operate in a networked environment using logical connections through the network interface 2070 to one or more other systems or computing machines across the network 2080. The network 2080 may include wide area networks (WAN), local area networks (LAN), intranets, the Internet, wireless access networks, wired networks, mobile networks, telephone networks, optical networks, or combinations thereof. The network 2080 may be packet switched, circuit switched, of any topology, and may use any communication protocol. Communication links within the network 2080 may involve various digital or an analog communication media such as fiber optic cables, free-space optics, waveguides, electrical conductors, wireless links, antennas, radio-frequency communications, and so forth.

The processor 2010 may be connected to the other elements of the computing machine 2000 or the various peripherals discussed herein through the system bus 2020. It should be appreciated that the system bus 2020 may be within the processor 2010, outside the processor 2010, or both. According to some embodiments, any of the processor 2010, the other elements of the computing machine 2000, or the various peripherals discussed herein may be integrated into a single device such as a system on chip (“SOC”), system on package (“SOP”), or ASIC device.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions. Further, a skilled programmer would be able to write such a computer program to implement an embodiment of the disclosed embodiments based on the appended flow charts and associated description in the application text. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

The example embodiments described herein can be used with computer hardware and software that perform the methods and processing functions described herein. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

Further embodiments are illustrated in the following Examples which are given for illustrative purposes only and are not intended to limit the scope of the invention.

### Optimized Cell Culture Conditions and Uses Thereof

Also described herein are optimized cell culture conditions for any particular sample that can be cultured and analyzed suing the combinatorial addressable array, models, and methods described herein. Such optimized conditions can be used to culture the samples such that they can be used in, among other things, in vitro assays to facilitate development of personalized and targeted therapeutics.

In some embodiments, described herein are cell culture conditions effective to modify a characteristic of a biological sample during culture comprising: a cell culture condition identified by performing a method of using the combinatorial addressable array, trained models, and/or computer implemented methods described elsewhere herein. Modification in this context refers to any measurable change in the characteristic being evaluated. In some embodiments, the change is a statistically significant change or a change that crosses a threshold value. In some embodiments, the change is an increase or a decrease.

In some embodiments, described herein are methods of creating a cell line or organoid, the method comprising: culturing a sample isolated from a subject in (a) one or more culture conditions of described elsewhere herein; (b) a combinatorial addressable array as described in greater detail elsewhere herein; or (c) a combination thereof. The method can further include measuring a characteristic of a cultured cell, optionally applying a trained model described elsewhere herein to choose cell culture conditions, and/or optionally training a machine learning or statistical model using the measurement of one or more characteristic of the cultured sample. In some embodiments the sample can contain a cell or cells. In some embodiments, the cell or cells cultured can form a spheroid, an organoid, a spheroid, a cell suspension model, an adherent cell model, or a combination thereof. In some embodiments, the sample and/or the cell or cells isolated from the subject is/are a cancer cell(s).

Exemplary culture techniques generally known in the art can be used in connection with determining the optimal culture conditions described herein and using the optimized culture conditions to, for example, culture a sample and/or progeny thereof for subsequent in vitro assays. The subsequent in vitro assays can be used, without limitation, to further research the sample and develop personalized therapies. In some embodiments, culturing comprises passaging the cells one or more times. In some embodiments, culturing does not comprise passaging. In some embodiments, culturing comprises expanding the cells. As used herein, “culturing” refers to maintaining cells under conditions in which they can proliferate and avoid senescence as a group of cells. “Culturing” can also include conditions in which the cells also or alternatively differentiate or change in some way, such as gaining or losing one or more functionalities and/or expressing different signatures. The term of art “passaging” refers to the removal of the medium and transfer of cells from a previous culture into fresh growth medium. Passaging differs from a simple media change, freshen or exchange. In the case of adherent cells, passaging includes detaching the cells from the culture dish or vessel surface, typically by enzymatic release such as trypsinization. Culture conditions that can be optimized included, media, pH, salinity, osmolarity, growth factors, antibacterials, antifungals, serum used, culture type etc. Other culture conditions are discussed elsewhere herein and can be optimized as discussed here.

### EXAMPLES

### Example 1—High-Throughput Empiric Culture Strategy for Optimizing Culture Conditions

Generally, conventional methods for evaluating a patient's sample only allow for a few culture conditions to be evaluated. This can be particularly problematic for limited samples. This has resulted in poor success with analyzing these samples and development of personalized and targeted therapies for patients. The high-throughput empiric culture strategies demonstrated herein can significantly increase the rate at which optimized culture conditions for a sample can be determined. This system is also referred to as the “HYBRID system” herein.

FIG. 1 shows a general workflow comparison between conventional cell culture analysis and embodiments of a high-throughput combinatorial assay described herein. FIGS. 2-3 show exemplary high-throughput combinatorial addressable array employing an empirical and dual media strategy. The high-throughput combinatorial addressable array can increase the success rate in identifying suitable culture conditions.

Over the last about 1.5 years, more than 37 genomically confirmed rare tumor models have been generated. FIG. 4 shows genomically confirmed rare tumor models generated using optimized culture conditions identified using embodiments of the high-throughput combinatorial addressable arrays and/or statistical and/or machine learning models described herein, which were then used to develop tumor models.

FIG. 5 shows exemplary samples by tumor type developed using optimal culture conditions identified using embodiments of the high-throughput combinatorial addressable arrays and/or statistical and/or machine learning models described herein.

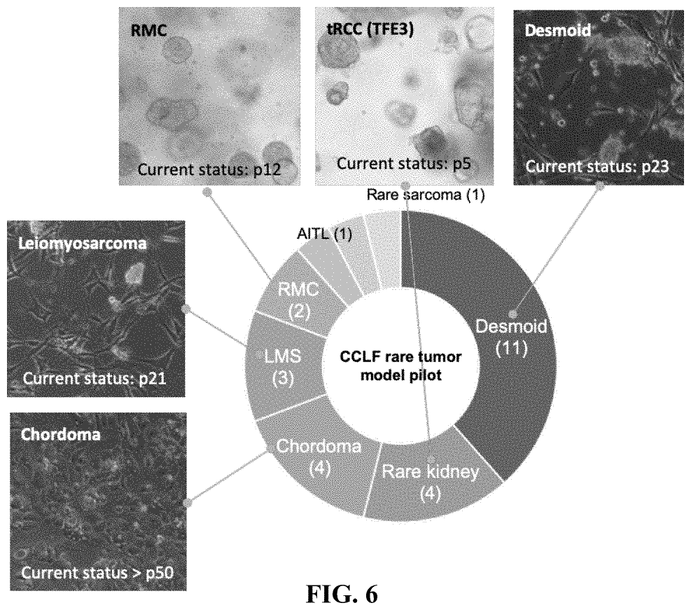

FIG. 6 shows exemplary rare tumor models generated using optimized culture conditions identified using embodiments of the high-throughput combinatorial addressable arrays and/or statistical and/or machine learning models described herein, which were then used to develop tumor models.

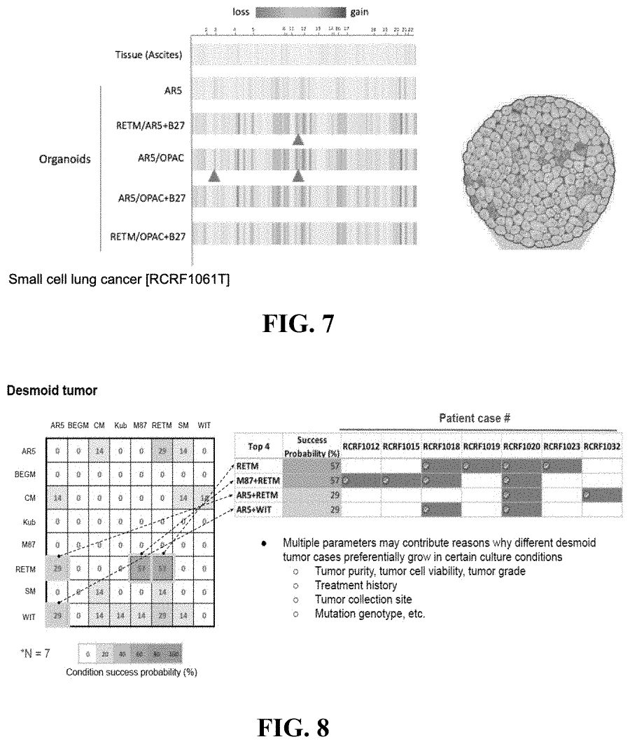

It was examined if different culture conditions would support propagation of different subclonal populations. FIG. 7 shows that different cell culture conditions can support propagation of different subclonal populations. FIG. 8 shows that different cell culture conditions supported different desmoid tumors to grow.

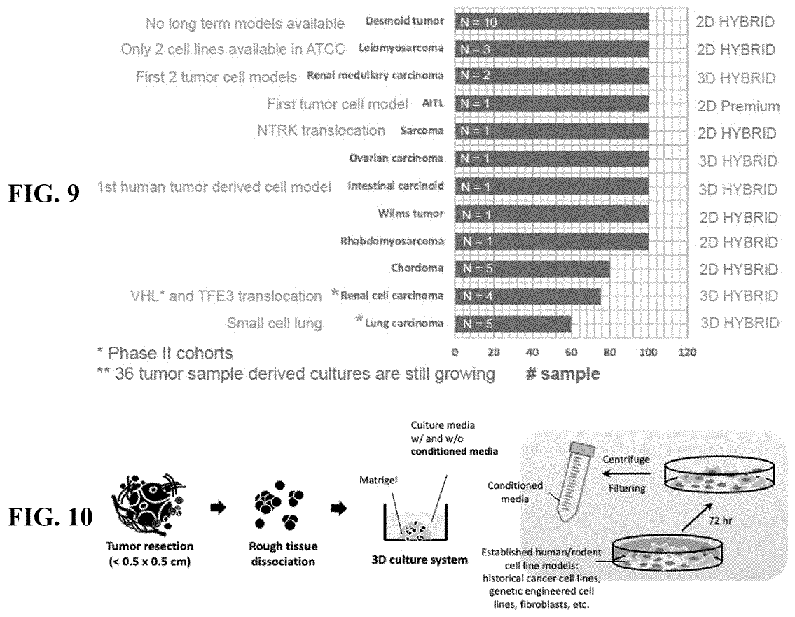

FIG. 9 shows model generation success broken down by tumor type.

Conditioned media can provide multiple different cellular factors at a time and can be used as a culture condition to subject a sample to. FIG. 10 shows exemplary development and collection of conditioned media from robust growing cell lines. Conditioned media can contain various bioactive factors (e.g., cytokines, growth factors, ECMs, etc.). In some embodiments, established cancer cell line collections, such as historical cancer cell lines and genetically engineered cell line model, can be used to generate conditioned media. Conditioned media can be used in connection with the HYBRID system and methods described herein as shown in FIG. 10.

### Example 3—Machine Learning for In Vitro Tumor Growth and Culture Conditions

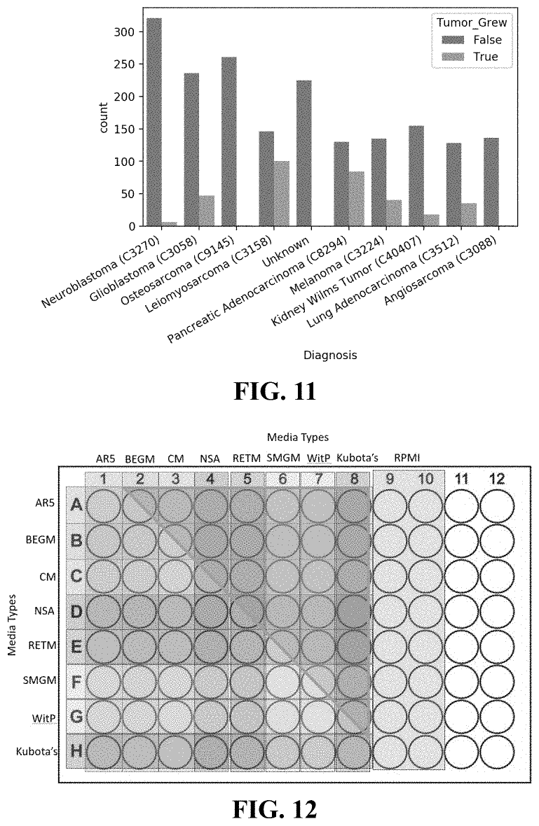

Although some success has been achieved with some cell lines (some approaching 60% success rate), many still have less than a 5% growth success rate. This means there is much wasted efforts and resources on conditions and techniques that are not working for many cell types and patients. Further, in many cases, the samples are limited in material and thus such waste can severely hinder the success of treatment as without in vitro patient samples, then practitioners must rely on population outcomes (which may or may not apply) and in some cases random chance. FIG. 11 can demonstrate tumor growth per diagnosis. The HYBRID technology is a matrix of different culture conditions and as discussed and demonstrated elsewhere herein. As is further discussed, it can be used to determine and predict growth conditions for a sample. FIG. 12 shows an exemplary combinatorial addressable array that contains a matrix of different media types, that can optionally be selected using a trained statistical or machine learning model. This can reduce the cost, labor, and amount of sample needed while increasing the success rate of achieving viable cell growth, particularly for rare and difficult cell types.

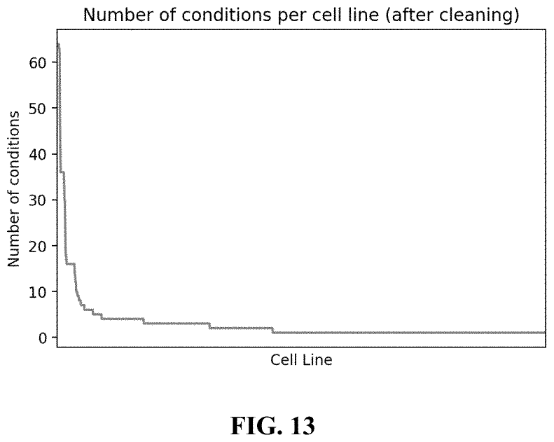

It was assessed if a machine-implemented model could be used to predict sample growth. Information is available about a sample, including clinical annotation (see also e.g., Appendix A of U.S. Provisional Application Ser. No. 63/057,812, which is incorporated by reference as if expressed in its entirety herein), and culture conditions. FIG. 13 shows the number of conditions per cell line after data cleaning. Information available to predict tumor growth were clinical annotations and culture conditions. The clinical annotations included tissue site, tumor type, and many others. The culture conditions tested were various (hundreds were tested) and included, for example, culture type (e.g., 2D or 3D), media type, etc. The HYBRID array/methodology required few samples to test many conditions (16 samples to test 64 different culture conditions) while the standard methodology required many samples to evaluate only 1-4 different culture conditions. Source of the data was divided by 1-4. Total raw data: 10,000 samples, 100 features (real time input in LIMS). Total cleaned data: 4500 samples, 14 features. Data was L-shaped. LIMS was used in conjunction with BSP and JIRA. Clinical features included cohort, diagnosis, primary disease, material type (e.g., fresh tissue, needle biopsy, blood, or cryopreserved), tissue site, tumor type (e.g., primary, metastasis, etc.), date and time of tumor collection. Culture condition features included flask coating, growth properties (e.g., 2D, 3D, and/or suspension), incubation condition (e.g., regular or hypoxia), media type (at initiation), starting media condition (native, 50/50 native/conditioned), 50/50 native/conditioned with supplementation).

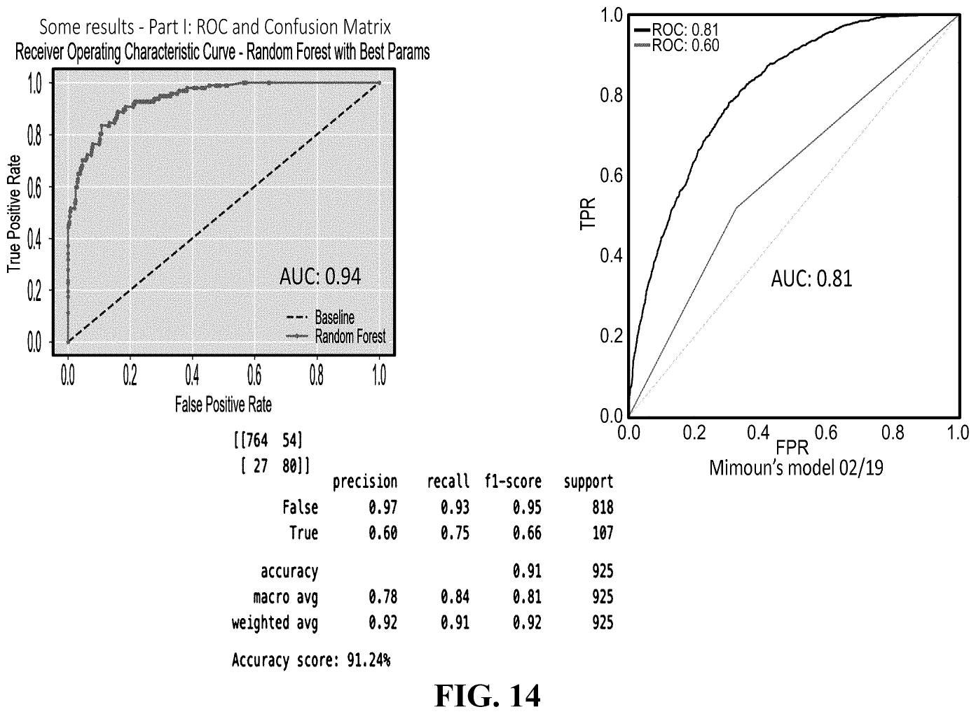

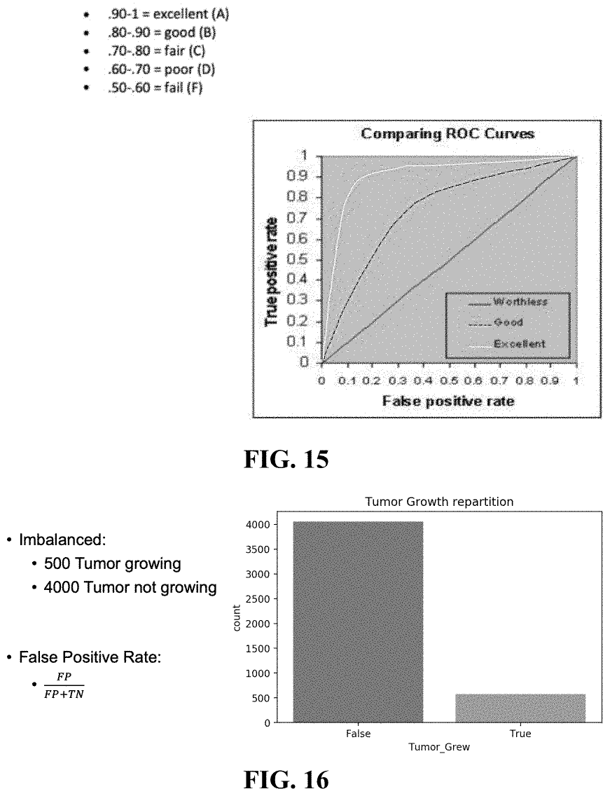

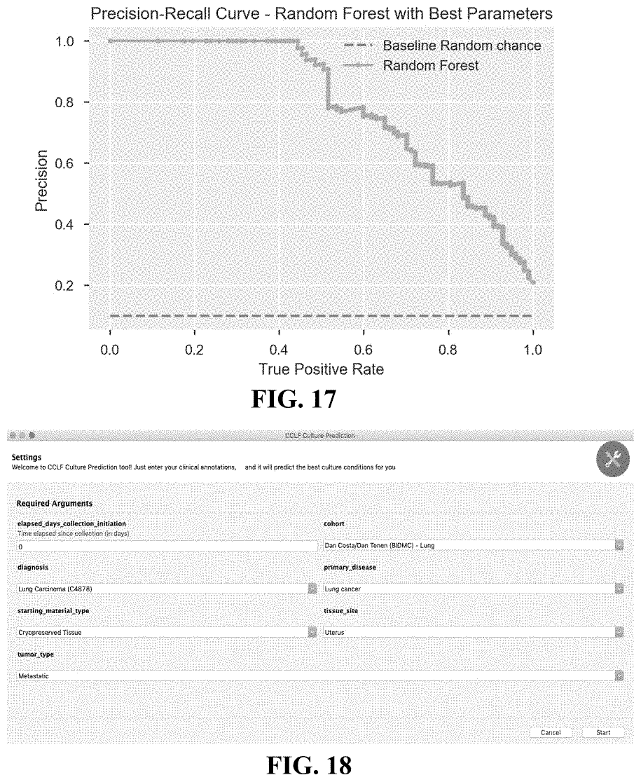

Consistent model training was applied. Methods incorporated in the training included train test split, OneHotEncoding or LabelEncoding, and grid search for best parameters with cross validation. Evaluation was performed using different types of models including logistic regression, decision tree analysis, random forests analysis and custom Bayesian models. FIG. 14 shows the model performance as demonstrated by ROC and Confusion Matrix. FIG. 15 shows a rough guide for classifying the accuracy of a diagnostic test based on the traditional academic point system. FIG. 15 shows three ROC curves representing excellent, good, and poor tests plotted on the same graph. The accuracy of the test depends on how well the test separates the group being tested into those with and without the disease or condition in question. Accuracy is measured by the area under the ROC curve. An area of 1 represents a perfect test. An area of 0.5 represents a poor test that does not provide any useful information. FIG. 16 shows imbalanced data. FIG. 17 shows a precision recall curve. AUC was about 70%. Precision was constant because recensions will always be the same whether 10 or 1000 items are classified.

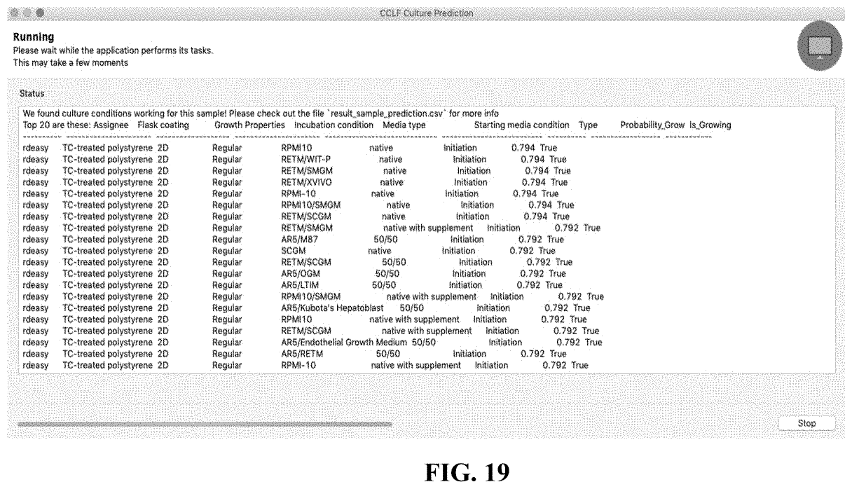

FIG. 18 shows a screen shot of cell culture prediction algorithm tool clinical annotation input page. Clinical annotations can be input by a user and the statistical or trained learning algorithm will determine culture conditions based upon the clinical annotation and other inputs to provide recommended culture conditions for that particular sample. FIG. 19 shows a screen shot of data output from the cell culture prediction algorithm.

### Example 4—Model Expansion Optimization

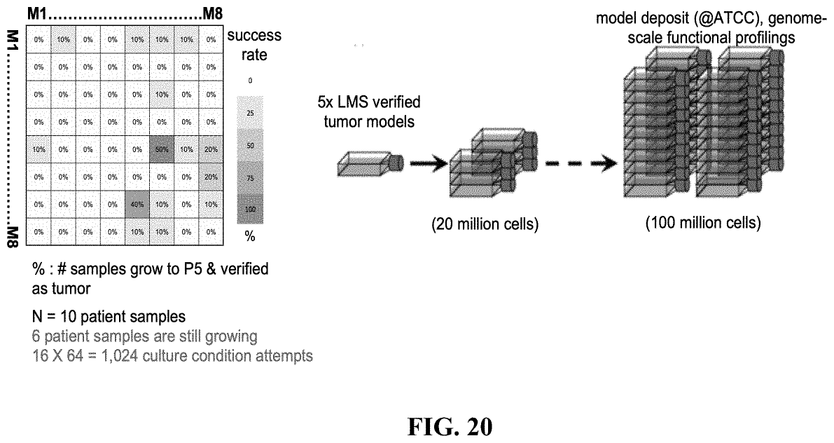

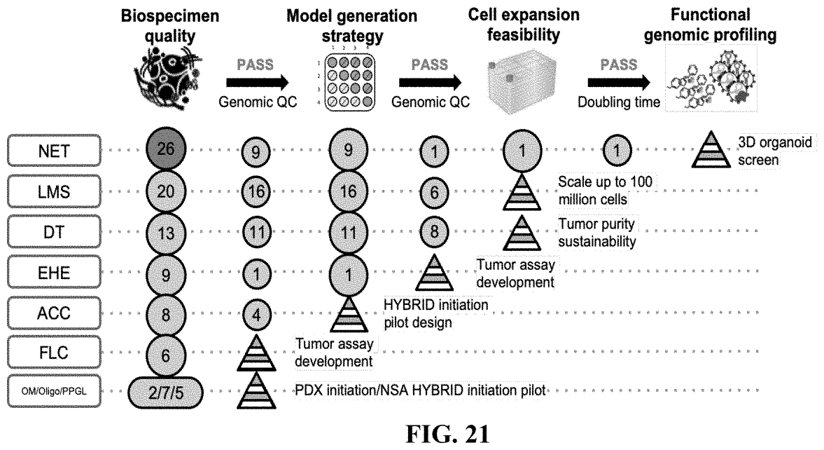

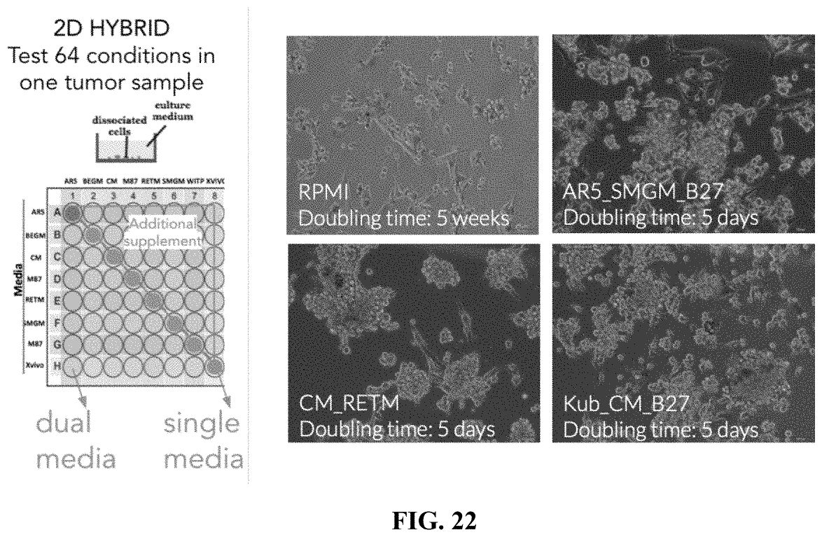

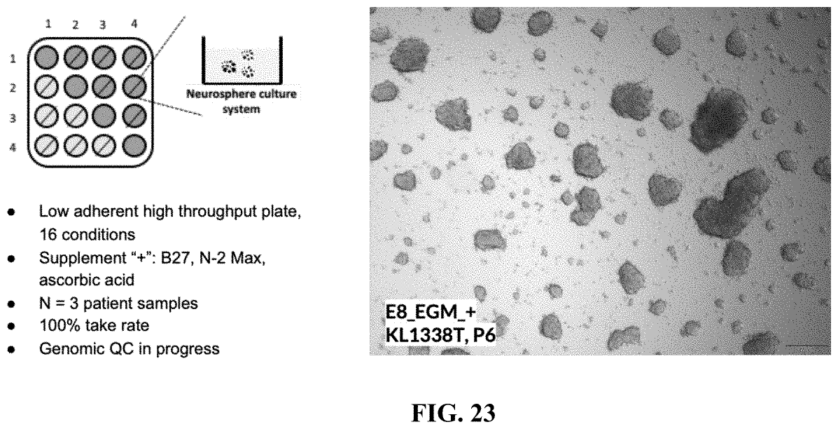

This Example can demonstrate model expansion optimization through a paracrine support screen for LMS model propagation as outlined in FIG. 20. FIG. 21 shows a strategy for onboarding nine major cohorts to generate a rare cancer dependency map. FIG. 22 shows that HYBRID technology can reduce doubling time of OM established cell lines. FIG. 23 shows the generation of a brain tumor model utilizing an embodiment of a high-throughput combinatorial addressable array and technique(s) as described herein and a neurosphere culture (e.g., medulloblastoma).

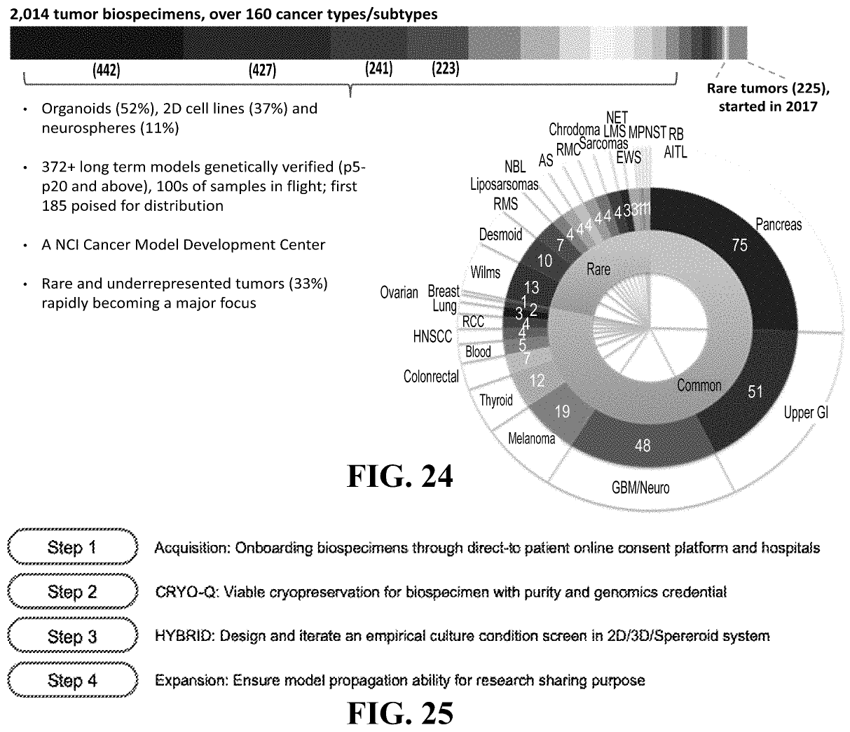

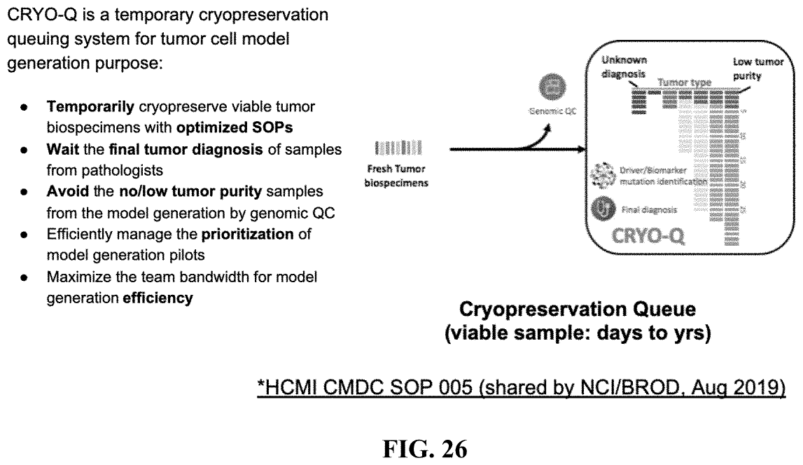

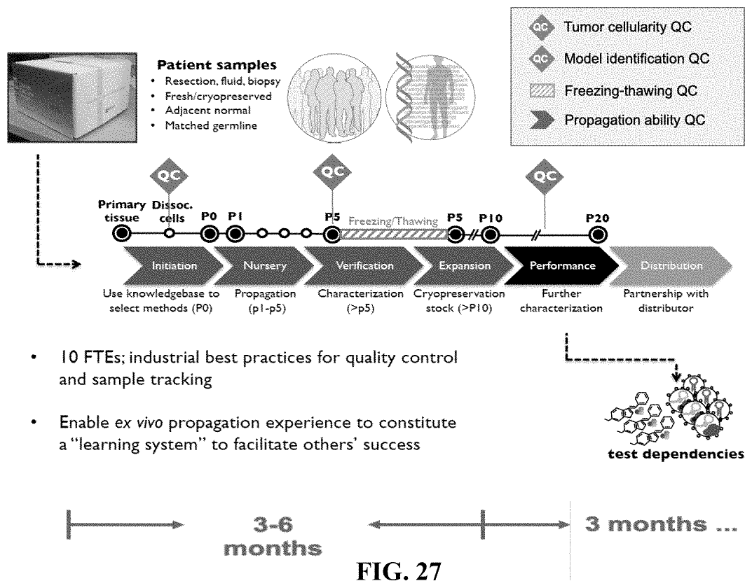

The HYBRID technology can be used to generate model cell, organoid, and tissue lines through the Cancer Cell Line Factory and for other repositories. FIG. 24 shows an overview of the Cancer Cell Line Factory (CCLF) which has developed organoids, 2D cell lines and neurospheres as and for the development of patient models. CCLF has developed over 37 long-term genetically verified (p5-p20 and above), 100s of samples in flight. Currently, organoids represent 52% of lines developed, 2D cell lines represent about 37% of lines developed and neurospheres represent about 11% of cell lines developed. FIG. 25 shows steps in developing a rare cancer dependency map. FIG. 26 shows the CRYO-Q workflow. CRYO-Q is a temporary cryopreservation queuing system for tumor cell model generation. It can be used in connection with the HYBRID technology described herein. FIG. 27 shows the workflow of generating a model cell, tissue, or organoid line at CCLF. Success is considered when growing cells can be passaged at least 5 times with genomic verification.

### Example 5—Verified Cancer Models Generated Using the HYBRID Approach

The combinatorial addressable array and analysis methods described herein and also referred to herein as HYBRID were used to generate cancer models. These exemplary verified cell models are shown in Table 2. A glossary of terms used in Table 2 are shown in Table 3.

TABLE 2
Verified Cancer Models Generated using HYBRID

| | Arxspan ID, | CCLF Publication ID, | | |
|---|---|---|---|---|
| CCLF Model ID | if applicable | if applicable | Organ System | Lineage |
| BID011T | TBD | TBD | Respiratory Tract | Lung |
| BID018T_ASC | TBD | TBD | Respiratory Tract | Lung |
| CCLF_BU1012T | TBD | TBD | Upper Aerodigestive | Head and Neck |
| | | | Tract | |
| CCLF_BU1017T | ACH-002867 | CCLF_LUPA_0001_T | Respiratory Tract | Lung |
| CCLF_cRCRF1018T | TBD | CCLF_UPGI_0055_T | Skin | Skin |
| CCLF_cRCRF1082T | TBD | CCLF_UPGI_0064_T | Skin | Skin |
| CCLF_cRCRF1084T | TBD | TBD | Urinary System | Kidney |
| CCLF_cRCRF1084T | TBD | TBD | Urinary System | Kidney |
| CCLF_cRCRF1086T | TBD | TBD | Reproductive System | Uterus |
| CCLF_CY1002T | TBD | CCLF_MELM_0013_T | Skin | Skin |
| CCLF_CY1005T | ACH-002770 | CCLF_MELM_0015_T | Skin | Skin |
| CCLF_CY1006T | TBD | CCLF_NEURO_0084_T | Skin | Skin |
| CCLF_CY1008T | ACH-002771 | CCLF_MELM_0016_T | Skin | Skin |
| CCLF_CY1009T | ACH-002772 | CCLF_MELM_0017_T | Skin | Skin |
| CCLF_CY1010T | ACH-002773 | CCLF_MELM_0018_T | Skin | Skin |
| CCLF_CY1010T | TBD | CCLF_MELM_0020_T | Skin | Skin |
| CCLF_CY1010T | TBD | CCLF_MELM_0021_T | Skin | Skin |
| CCLF_CY1013T | TBD | CCLF_NEURO_0085_T | Skin | Skin |
| CCLF_CY1018T | TBD | CCLF_NEURO_0087_T | Skin | Skin |
| CCLF_CY1020T | TBD | TBD | Skin | Skin |
| CCLF_CY1025T | TBD | CCLF_NEURO_0090_T | Skin | Skin |
| CCLF_CY1026T | TBD | TBD | Skin | Skin |
| CCLF_CY1028T | TBD | TBD | Skin | Skin |
| CCLF_CY1029T | TBD | TBD | Skin | Skin |
| CCLF_CY1030T | TBD | TBD | Skin | Skin |
| CCLF_CY1032T | TBD | TBD | Skin | Skin |
| CCLF_CY1033T | TBD | TBD | Skin | Skin |
| CCLF_CY1034T | TBD | TBD | Skin | Skin |
| CCLF_CY1035T | TBD | TBD | Skin | Skin |
| CCLF_CY1036T | TBD | CCLF_NEURO_0091_T | Skin | Skin |
| CCLF_CY1037T | TBD | CCLF_NEURO_0092_T | Skin | Skin |
| CCLF_CY1038T | TBD | CCLF_NEURO_0093_T | Skin | Skin |
| CCLF_CY1039T | TBD | CCLF_NEURO_0094_T | Skin | Skin |
| CCLF_CY1040T | TBD | CCLF_MELM_0022_T | Skin | Skin |
| CCLF_CY1041T | TBD | CCLF_MELM_0023_T | Skin | Skin |
| CCLF_CY1043T | TBD | TBD | Skin | Skin |
| CCLF_CY1046T | TBD | CCLF_MELM_0024_T | Skin | Skin |
| CCLF_KL1017T | TBD | CCLF_NEURO_0020_T | Connective and Soft | Soft Tissue |
| | | | Tissue | |
| CCLF_KL1051T | TBD | TBD | Skin | Skin |
| CCLF_KL1100T | TBD | TBD | Nervous System | Central Nervous |
| | | | | System |
| CCLF_KL1105T | TBD | CCLF_NEURO_0042_T | Skin | Skin |
| CCLF_KL1117T | TBD | CCLF_NEURO_0043_T | Respiratory Tract | Lung |
| CCLF_KL1122T | ACH-002457 | CCLF_NEURO_0037_T | Nervous System | Central Nervous |
| | | | | System |
| CCLF_KL1131T | TBD | TBD | Skin | Skin |
| CCLF_KL1185T | TBD | TBD | Skin | Skin |
| CCLF_KL1187T | TBD | CCLF_NEURO_0049_T | Skin | Skin |
| CCLF_KL1194T | TBD | TBD | Nervous System | Central Nervous |
| | | | | System |
| CCLF_KL1197T | TBD | CCLF_NEURO_0060_T | Respiratory Tract | Lung |
| CCLF_KL1198T | TBD | TBD | Respiratory Tract | Lung |
| CCLF_KL1212T | TBD | TBD | Nervous System | Central Nervous |
| | | | | System |
| CCLF_KL1214T | TBD | TBD | Respiratory Tract | Lung |
| CCLF_KL1217T | TBD | CCLF_NEURO_0062_T | Skin | Skin |
| CCLF_KL1235T | TBD | CCLF_NEURO_0065_T | Respiratory Tract | Lung |
| CCLF_KL1244T | TBD | CCLF_MELM_0025_T | Skin | Skin |
| CCLF_KL1270T | ACH-002752 | CCLF_NEURO_0085_T | Respiratory Tract | Lung |
| CCLF_KL1272T | TBD | CCLF_NEURO_0073_T | Skin | Skin |
| CCLF_KL1273T | TBD | TBD | Respiratory Tract | Lung |
| CCLF_KL1273T | TBD | TBD | Respiratory Tract | Lung |
| CCLF_KL1274T | ACH-002866 | CCLF_NEURO_0096_T | Urinary System | Kidney |
| CCLF_KL1274T | TBD | TBD | Urinary System | Kidney |
| CCLF_KL1282T | TBD | CCLF_NEURO_0074_T | Reproductive System | Uterus |
| CCLF_KL1283T | ACH-002769 | CCLF_NEURO_0084_T | Respiratory Tract | Lung |
| CCLF_KL1308T | ACH-002775 | CCLF_NEURO_0083_T | Skin | Skin |
| CCLF_KL1328T | ACH-002774 | CCLF_NEURO_0082_T | Skin | Skin |
| CCLF_KL1332T | TBD | TBD | Respiratory Tract | Lung |
| CCLF_KL1338T | TBD | CCLF_NEURO_0095_T | Nervous System | Central Nervous |
| | | | | System |
| CCLF_KL1345T | TBD | TBD | Respiratory Tract | Lung |
| CCLF_KL1345T | TBD | TBD | Respiratory Tract | Lung |
| CCLF_KL1361T | TBD | CCLF_NEURO_0089_T | Nervous System | Central Nervous |
| | | | | System |
| CCLF_KL1374T | TBD | TBD | Skin | Skin |
| CCLF_KL1412T | TBD | TBD | Skin | Skin |
| CCLF_KL1412T | TBD | CCLF_MELM_0026_T | Skin | Skin |
| CCLF_PEDS1011T | TBD | TBD | Urinary System | Kidney |
| CCLF_PEDS1012T | ACH-002438 | CCLF_PEDS_0026_T | Urinary System | Kidney |
| CCLF_PEDS1041T | TBD | CCLF_PEDS_0034_T | Connective and Soft | Soft Tissue |
| | | | Tissue | |
| CCLF_PEDS1046T | TBD | CCLF_PEDS_0035_T | Connective and Soft | Soft Tissue |
| | | | Tissue | |
| CCLF_PEDS1064T | TBD | CCLF_PEDS_0036_T | Connective and Soft | Soft Tissue |
| | | | Tissue | |
| CCLF_PEDS1065T | TBD | CCLF_PEDS_0037_T | Connective and Soft | Soft Tissue |
| | | | Tissue | |
| CCLF_PEDS1094T | TBD | CCLF_PEDS_0038_T | Urinary System | Kidney |
| CCLF_PEDS1113T | TBD | CCLF_PEDS_0039_T | Urinary System | Kidney |
| CCLF_PEDS1198T | TBD | TBD | Urinary System | Kidney |
| CCLF_RCRF1008T | TBD | TBD | Connective and Soft | Bone |
| | | | Tissue | |
| CCLF_RCRF1009T | TBD | CCLF_RARE_0001_T | Urinary System | Kidney |
| CCLF_RCRF1015T | TBD | CCLF_RARE_0003_T | Connective and Soft | Soft Tissue |
| | | | Tissue | |
| CCLF_RCRF1018T | ACH-002843 | TBD | Connective and Soft | Soft Tissue |
| | | | Tissue | |
| CCLF_RCRF1025T | TBD | CCLF_RARE_0004_T | Connective and Soft | Soft Tissue |
| | | | Tissue | |
| CCLF_RCRF1033T | TBD | CCLF_RARE_0006_T | Connective and Soft | Soft Tissue |
| | | | Tissue | |
| CCLF_RCRF1049T | TBD | CCLF_RARE_0007_T | Digestive System | Intestine |
| CCLF_RCRF1052T | TBD | TBD | Connective and Soft | Bone |
| | | | Tissue | |
| CCLF_RCRF1061T | TBD | CCLF_LUPA_0002_T | Respiratory Tract | Lung |
| CCLF_RCRF1072T | TBD | TBD | Integumentary System | Eye |
| CCLF_SK1006T | TBD | TBD | Skin | Skin |
| CCLF_SK1018T | TBD | TBD | Skin | Skin |
| CCLF_SS1002T | TBD | CCLF_KIPA_0001_T | Urinary System | Kidney |
| CCLF_SS1009T | TBD | CCLF_KIPA_0002_T | Urinary System | Kidney |
| CCLF_SS1010T | TBD | TBD | Urinary System | Kidney |
| CCLF_SS1013T | TBD | TBD | Urinary System | Kidney |
| CCLF_SS1016T | TBD | TBD | Urinary System | Kidney |
| CCLF_SS1018T | TBD | TBD | Urinary System | Kidney |
| CCLF_SS1022T | TBD | TBD | Urinary System | Kidney |
| CCLF_SS1035T | TBD | TBD | Urinary System | Kidney |
| CCLF_SS1036T | TBD | CCLF_KIPA_0003_T | Urinary System | Kidney |
| CCLF_SS1041T | TBD | TBD | Urinary System | Kidney |
| CCLF_SS1044T | TBD | TBD | Urinary System | Kidney |
| CY001T | ACH-002403 | CCLF_MELM_0001_T | Skin | Skin |
| CY002T | ACH-002404 | CCLF_MELM_0002_T | Skin | Skin |
| CY003T_sup | ACH-002405 | CCLF_MELM_0003_T | Skin | Skin |
| CY004T | TBD | CCLF_MELM_0004_T | Skin | Skin |
| CY006T | ACH-002407 | CCLF_MELM_0005_T | Skin | Skin |
| CY014T | TBD | CCLF_MELM_0007_T | Skin | Skin |
| CY016T | TBD | TBD | Skin | Skin |
| CY018T | TBD | CCLF_MELM_0010_T | Skin | Skin |
| CY020T | TBD | CCLF_MELM_0011_T | Skin | Skin |
| CY025T_1 | TBD | CCLF_MELM_0012_T | Skin | Skin |
| CY025T_2 | TBD | CCLF_MELM_0012_T2 | Skin | Skin |
| DW019T | TBD | CCLF_HEME_0001_T | Hematopoietic and | Lymphocyte |
| | | | Lymphoid System | |
| DW020T | TBD | CCLF_HEME_0002_T | Hematopoietic and | Lymphocyte |
| | | | Lymphoid System | |
| DW023T | TBD | CCLF_HEME_0003_T | Hematopoietic and | Lymphocyte |
| | | | Lymphoid System | |
| DW031T | TBD | TBD | Hematopoietic and | Lymphocyte |
| | | | Lymphoid System | |
| DW033T | TBD | TBD | Hematopoietic and | Lymphocyte |
| | | | Lymphoid System | |
| DW048T | TBD | TBD | Hematopoietic and | Blood |
| | | | Lymphoid System | |
| EH07T16 | TBD | TBD | Urinary System | Kidney |
| EH07T16 | TBD | TBD | Urinary System | Kidney |
| EH09T | TBD | TBD | Urinary System | Kidney |
| EW009T | TBD | TBD | Connective and Soft | Soft Tissue |
| | | | Tissue | |
| EW010T | TBD | TBD | Connective and Soft | Soft Tissue |
| | | | Tissue | |
| EW012T | TBD | TBD | Connective and Soft | Soft Tissue |
| | | | Tissue | |
| HG002T | TBD | TBD | Upper Aerodigestive | Head and Neck |
| | | | Tract | |
| HG011T | TBD | CCLF_NPDX_0002_T | Connective and Soft | Soft Tissue |
| | | | Tissue | |
| JL16 | TBD | CCLF_THYR_0001_T | Head and Neck | Thyroid |
| JL27 | TBD | CCLF_THYR_0002_T | Head and Neck | Thyroid |
| JL30 | TBD | CCLF_THYR_0003_T | Head and Neck | Thyroid |
| JL36 | TBD | CCLF_THYR_0004_T | Head and Neck | Thyroid |
| JL37 | TBD | CCLF_THYR_0005_T | Head and Neck | Thyroid |
| JL42 | TBD | CCLF_THYR_0006_T | Head and Neck | Thyroid |
| JL48T | TBD | TBD | Head and Neck | Thyroid |
| JL50T_PF2 | ACH-002440 | CCLF_THYR_0008_T | Head and Neck | Thyroid |
| PEDS005T_PF_AD | TBD | TBD | Urinary System | Kidney |
| PEDS005T_PF_SUS | TBD | CCLF_PEDS_0045_T | Urinary System | Kidney |
| PEDS012T_P | ACH-001423 | CCLF_PEDS_0002_T | Urinary System | Kidney |
| PEDS015T | ACH-001164 | CCLF_PEDS_0003_T | Connective and Soft | Soft Tissue |
| | | | Tissue | |
| PEDS018T | TBD | CCLF_PEDS_0044_T | Connective and Soft | Soft Tissue |
| | | | Tissue | |
| PEDS022T | TBD | TBD | Urinary System | Kidney |
| PEDS040T | TBD | TBD | Urinary System | Kidney |
| PEDS051T | TBD | TBD | Connective and Soft | Soft Tissue |
| | | | Tissue | |
| PEDS063T | TBD | TBD | Urinary System | Kidney |
| PEDS078T_1 | ACH-001427 | CCLF_PEDS_0007_T1 | Connective and Soft | Bone |
| | | | Tissue | |
| PEDS092T_PF | ACH-001433 | CCLF_PEDS_0008_T | Connective and Soft | Soft Tissue |
| | | | Tissue | |
| PEDS109T_AT | TBD | TBD | Urinary System | Kidney |
| PEDS110T_PF | ACH-001429 | CCLF_PEDS_0009_T | Connective and Soft | Bone |
| | | | Tissue | |
| PEDS117T_PF | ACH-001428 | CCLF_PEDS_0010_T | Connective and Soft | Bone |
| | | | Tissue | |
| PEDS118T | TBD | CCLF_PEDS_0042_T | Connective and Soft | Endothelial Cells |
| | | | Tissue | |
| PEDS145T_PDX | TBD | TBD | Urinary System | Kidney |
| PEDS149T | TBD | CCLF_PEDS_0013_T | Connective and Soft | Soft Tissue |
| | | | Tissue | |
| PEDS157T_AT | ACH-002431 | CCLF_PEDS_0014_T1 | Nervous System | Peripheral |
| | | | | Nervous System |
| PEDS160T | TBD | TBD | Urinary System | Kidney |
| PEDS172T_PF | TBD | CCLF_PEDS_0043_T | Connective and Soft | Soft Tissue |
| | | | Tissue | |
| PEDS182T | ACH-002433 | CCLF_PEDS_0019_T | Connective and Soft | Bone |
| | | | Tissue | |
| PEDS187T | TBD | CCLF_PEDS_0020_T | Urinary System | Kidney |
| PEDS195T_4_PF | TBD | TBD | Nervous System | Peripheral |
| | | | | Nervous System |
| PEDS196T | ACH-002435 | CCLF_PEDS_0022_T | Urinary System | Kidney |
| PEDS204T | ACH-002436 | CCLF_PEDS_0023_T | Urinary System | Kidney |
| SM045T_PrimWITP | TBD | TBD | Reproductive System | Uterus |
| SP020T | TBD | TBD | Upper Aerodigestive | Head and Neck |
| | | | Tract | |
| SP022T | TBD | CCLF_HNSC_0001_T | Upper Aerodigestive | Head and Neck |
| | | | Tract | |
| SP025T_P | TBD | CCLF_HNSC_0002_T | Upper Aerodigestive | Head and Neck |
| | | | Tract | |
| CCLF_BU1037T | TBD | | Respiratory Tract | Lung |
| CCLF_BU1037T | TBD | | Respiratory Tract | Lung |
| CCLF_BU1043T | TBD | | Respiratory Tract | Lung |
| CCLF_BU1043T | TBD | | Respiratory Tract | Lung |
| CCLF_BU1043T | TBD | | Respiratory Tract | Lung |
| CCLF_cRCRF1073T | TBD | | Respiratory Tract | Lung |
| CCLF_cRCRF1074T | TBD | | Respiratory Tract | Lung |
| CCLF_cRCRF1074T | TBD | | Respiratory Tract | Lung |
| CCLF_cRCRF1074T | TBD | | Respiratory Tract | Lung |
| CCLF_cRCRF1074T | TBD | | Respiratory Tract | Lung |
| CCLF_cRCRF1074T | TBD | | Respiratory Tract | Lung |
| CCLF_CY1046T | TBD | | Skin | Skin |
| CCLF_GC1004T | TBD | | Connective and Soft | Soft Tissue |
| | | | Tissue | |
| CCLF_GC1004T | TBD | | Connective and Soft | Soft Tissue |
| | | | Tissue | |
| CCLF_GC1004T | TBD | | Connective and Soft | Soft Tissue |
| | | | Tissue | |
| CCLF_JL1003T | TBD | | Head and Neck | Thyroid |
| CCLF_KF1002T | TBD | | | |
| CCLF_KF1002T | TBD | | | |
| CCLF_KF1002T | TBD | | | |
| CCLF_KF1002T | TBD | | | |
| CCLF_KL1445T | TBD | | Respiratory Tract | Lung |
| CCLF_KL1445T | TBD | | Respiratory Tract | Lung |
| CCLF_KL1445T | TBD | | Respiratory Tract | Lung |
| CCLF_MDA1016T | TBD | | Connective and Soft | Soft Tissue |
| | | | Tissue | |
| CCLF_MDA1019T | TBD | | Connective and Soft | Soft Tissue |
| | | | Tissue | |
| CCLF_MDA1019T | TBD | | Connective and Soft | Soft Tissue |
| | | | Tissue | |
| CCLF_MDA1019T | TBD | | Connective and Soft | Soft Tissue |
| | | | Tissue | |
| CCLF_MDA1019T | TBD | | Connective and Soft | Soft Tissue |
| | | | Tissue | |
| CCLF_MDA1019T | TBD | | Connective and Soft | Soft Tissue |
| | | | Tissue | |
| CCLF_PEDS1164T | TBD | | Urinary System | Kidney |
| CCLF_PEDS1164T | TBD | | Urinary System | Kidney |
| CCLF_PEDS1164T | TBD | | Urinary System | Kidney |
| CCLF_PEDS1164T | TBD | | Urinary System | Kidney |
| CCLF_PEDS1164T | TBD | | Urinary System | Kidney |
| CCLF_PEDS1164T | TBD | | Urinary System | Kidney |
| CCLF_PEDS1164T | TBD | | Urinary System | Kidney |
| CCLF_PEDS1164T | TBD | | Urinary System | Kidney |
| CCLF_RCRF1011T | TBD | | Connective and Soft | Soft Tissue |
| | | | Tissue | |
| CCLF_RCRF1011T | TBD | | Connective and Soft | Soft Tissue |
| | | | Tissue | |
| CCLF_RCRF1011T | TBD | | Connective and Soft | Soft Tissue |
| | | | Tissue | |
| CCLF_RCRF1022T | TBD | | Connective and Soft | Soft Tissue |
| | | | Tissue | |
| CCLF_RCRF1056T | TBD | | Connective and Soft | Soft Tissue |
| | | | Tissue | |
| CCLF_RCRF1070T | TBD | | Connective and Soft | Soft Tissue |
| | | | Tissue | |
| CCLF_RCRF1078T | TBD | | Connective and Soft | Soft Tissue |
| | | | Tissue | |
| CCLF_RCRF1103T | TBD | | Connective and Soft | Soft Tissue |
| | | | Tissue | |
| CCLF_RCRF1161T | TBD | | Connective and Soft | Soft Tissue |
| | | | Tissue | |
| CCLF_RCRF1161T | TBD | | Connective and Soft | Soft Tissue |
| | | | Tissue | |
| CCLF_SS1042T | TBD | | Urinary System | Kidney |
| CCLF_SS1066T | TBD | | Urinary System | Kidney |

| | | Rare or | | Short term or |
|---|---|---|---|---|
| | Diagnosis | Common Cancer | Verification | Long term |
| Diagnosis | Subtype | Model? | method | culture? |
| Lung Carcinoma | | Common | Genomic QC Panel | Long term |
| Lung Carcinoma | | Common | Genomic QC Panel | TBD |
| Head and Neck Squamous | | Common | Genomic QC Panel | Short term |
| Cell Carcinoma | | | | |
| Lung Carcinoma | | Common | Genomic QC Panel | Long term |
| Melanoma | | Common | Genomic QC Panel | Long term |
| Melanoma | | Common | Genomic QC Panel | Long term |
| Renal Cell Carcinoma | Non Clear Cell Renal Cell | Common | Genomic QC Panel | TBD |
| | Carcinoma | | | |
| Renal Cell Carcinoma | Non Clear Cell Renal Cell | Common | Genomic QC Panel | TBD |
| | Carcinoma | | | |
| Uterine Carcinoma | | Common | Genomic QC Panel | TBD |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | TBD |
| Melanoma | | Common | Genomic QC Panel | Long term |
| Melanoma | | Common | Genomic QC Panel | Long term |
| Melanoma | | Common | Genomic QC Panel | TBD |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | TBD |
| Melanoma | | Common | Genomic QC Panel | Long term |
| Spindle Cell Sarcoma | | Rare | Genomic QC Panel | Long term |
| Melanoma | | Common | Genomic QC Panel | Short term |
| Meningioma | Atypical Meningioma | Rare | Genomic QC Panel | Long term |
| Melanoma | | Common | Genomic QC Panel | Long term |
| Lung Carcinoma | | Common | Genomic QC Panel | Long term |
| Meningioma | Anaplastic Meningioma | Rare | Genomic QC Panel | Long term |
| Melanoma | | Common | Genomic QC Panel | Short term |
| Melanoma | | Common | Genomic QC Panel | Long term |
| Melanoma | | Common | Genomic QC Panel | Long term |
| Meningioma | Meningioma, WHO Grade I | Common | Genomic QC Panel | Long term |
| Lung Carcinoma | | Common | Genomic QC Panel | Long term |
| Lung Carcinoma | | Common | Genomic QC Panel | Long term |
| Meningioma | Meningioma, WHO Grade I | Rare | Genomic QC Panel | Long term |
| Lung Carcinoma | | Common | Genomic QC Panel | Short term |
| Melanoma | | Common | Genomic QC Panel | Long term |
| Lung Carcinoma | | Common | Genomic QC Panel | Long term |
| Melanoma | | Common | Genomic QC Panel | Long term |
| Lung Carcinoma | | Common | Genomic QC Panel | Short term |
| Melanoma | | Common | Genomic QC Panel | Long term |
| Lung Carcinoma | | Common | Genomic QC Panel | Short term |
| Lung Carcinoma | | Common | Genomic QC Panel | Short term |
| Renal Cell Carcinoma | Clear Cell Renal Cell | Common | Genomic QC Panel | Long term |
| | Carcinoma | | | |
| Renal Cell Carcinoma | Clear Cell Renal Cell | Common | Genomic QC Panel | TBD |
| | Carcinoma | | | |
| Mullerian Carcinoma | | Common | Genomic QC Panel | Short term |
| Lung Carcinoma | Small Cell Lung Cancer | Common | Genomic QC Panel | Short term |
| Melanoma | | Common | Genomic QC Panel | Long term |
| Melanoma | | Common | Genomic QC Panel | Long term |
| Lung Carcinoma | | Common | Genomic QC Panel | Short term |
| Medulloblastoma | | Rare | Genomic QC Panel | Long term |
| Lung Carcinoma | Small Cell Lung Cancer | Common | Genomic QC Panel | TBD |
| Lung Carcinoma | Small Cell Lung Cancer | Common | Genomic QC Panel | Short term |
| Glioma | Oligodendroglioma | Rare | Genomic QC Panel | Short term |
| Melanoma | | Common | Genomic QC Panel | Long term |
| Melanoma | | Common | Genomic QC Panel | Long term |
| Melanoma | | Common | Genomic QC Panel | Long term |
| Wilms Tumor | | Rare | Genomic QC Panel | Long term |
| Wilms Tumor | | Rare | Genomic QC Panel | Long term |
| Rhabdomyosarcoma | Alveolar | Rare | RT-PCR | Long term |
| | Rhabdomyosarcoma | | | |
| Rhabdomyosarcoma | Alveolar | Rare | RT-PCR | Long term |
| | Rhabdomyosarcoma | | | |
| Rhabdomyosarcoma | Alveolar | Rare | RT-PCR | Long term |
| | Rhabdomyosarcoma | | | |
| Rhabdomyosarcoma | Congenital/Infantile | Rare | RT-PCR | Long term |
| | Rhabdomyosarcoma | | | |
| Wilms Tumor | | Rare | Genomic QC Panel | Short term |
| Wilms Tumor | | Rare | Genomic QC Panel | Long term |
| Wilms Tumor | | Rare | Genomic QC Panel | Long term |
| Chordoma | | Rare | ELISA | Short term |
| Renal Cell Carcinoma | Renal Medullary Carcinoma | Rare | Genomic QC Panel | Long term |
| Desmoid Tumor | | Rare | Genomic QC Panel | Short term |
| Desmoid Tumor | | Rare | PCR | Long term |
| Leiomyosarcoma | | Rare | Genomic QC Panel | Long term |
| Leiomyosarcoma | | Rare | Genomic QC Panel | Long term |
| Neuroendocrine Tumor | Intestinal Carcinoid | Rare | Genomic QC Panel | Long term |
| Chordoma | | Rare | ELISA | Short term |
| Lung Carcinoma | Small Cell Lung Cancer | Common | Genomic QC Panel | Long term |
| Uveal Melanoma | | Rare | Genomic QC Panel | TBD |
| Melanoma | | Common | Genomic QC Panel | Long term |
| Melanoma | | Common | Genomic QC Panel | Long term |
| Renal Cell Carcinoma | Clear Cell Renal Cell | Common | Genomic QC Panel | Long term |
| | Carcinoma | | | |
| Renal Cell Carcinoma | Clear Cell Renal Cell | Common | Genomic QC Panel | Long term |
| | Carcinoma | | | |
| Renal Cell Carcinoma | Translocation Renal Cell | Rare | ELISA | Short term |
| | Carcinoma | | | |
| Renal Cell Carcinoma | Clear Cell Renal Cell | Common | Genomic QC Panel | TBD |
| | Carcinoma | | | |
| Renal Cell Carcinoma | Translocation Renal Cell | Rare | Genomic QC Panel | Long term |
| | Carcinoma | | | |
| Renal Cell Carcinoma | Chromophobe Renal Cell | Rare | Genomic QC Panel | Long term |
| | Carcinoma | | | |
| Renal Cell Carcinoma | Clear Cell Renal Cell | Common | Genomic QC Panel | TBD |
| | Carcinoma | | | |
| Renal Cell Carcinoma | Clear Cell Renal Cell | Common | Genomic QC Panel | Short term |
| | Carcinoma | | | |
| Renal Cell Carcinoma | Clear Cell Renal Cell | Common | Genomic QC Panel | Short term |
| | Carcinoma | | | |
| Renal Cell Carcinoma | Papillary Renal Cell | Rare | Genomic QC Panel | TBD |
| | Carcinoma | | | |
| Renal Cell Carcinoma | Clear Cell Renal Cell | Common | Genomic QC Panel | Short term |
| | Carcinoma | | | |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Slow-growing |
| Melanoma | | Common | Genomic QC Panel | Long term |
| Melanoma | | Common | Genomic QC Panel | Long term |
| Melanoma | | Common | Genomic QC Panel | Long term |
| Melanoma | | Common | Genomic QC Panel | Long term |
| Non-Hodgkin Lymphoma | Double Hit Diffuse Large B- | Rare | Genomic QC Panel | Long term |
| | Cell Lymphoma | | | |
| Non-Hodgkin Lymphoma | Double Hit Diffuse Large B- | Rare | Genomic QC Panel | Long term |
| | Cell Lymphoma | | | |
| Non-Hodgkin Lymphoma | Angioimmunoblastic T-Cell | Rare | Genomic QC Panel | Long term |
| | Lymphoma | | | |
| Non-Hodgkin Lymphoma | Peripheral T-Cell | Rare | Genomic QC Panel | Long term |
| | Lymphoma | | | |
| Non-Hodgkin Lymphoma | Peripheral T-Cell | Rare | Genomic QC Panel | Long term |
| | Lymphoma | | | |
| Prolymphocytic Leukemia | T-Cell Prolymphocytic | Rare | Genomic QC Panel | TBD |
| | Leukemia | | | |
| Angiomyolipoma | | Rare | Genomic QC Panel | Long term |
| Angiomyolipoma | | Rare | Genomic QC Panel | Long term |
| Angiomyolipoma | | Rare | Genomic QC Panel | TBD |
| Liposarcoma | | Rare | Genomic QC Panel | TBD |
| Liposarcoma | | Rare | Genomic QC Panel | Long term |
| Translocation Driven | NTRK Translocation Driven | Rare | Genomic QC Panel | TBD |
| Sarcoma | Sarcoma | | | |
| Head and Neck Squamous | | Common | Genomic QC Panel | Long term |
| Cell Carcinoma | | | | |
| Liposarcoma | | Rare | Genomic QC Panel | TBD |
| Thyroid Carcinoma | Anaplastic Thyroid | Rare | Genomic QC Panel | Long term |
| | Carcinoma | | | |
| Thyroid Carcinoma | Anaplastic Thyroid | Rare | Genomic QC Panel | Long term |
| | Carcinoma | | | |
| Thyroid Carcinoma | Anaplastic Thyroid | Rare | Genomic QC Panel | Long term |
| | Carcinoma | | | |
| Thyroid Carcinoma | Anaplastic Thyroid | Rare | Genomic QC Panel | Long term |
| | Carcinoma | | | |
| Thyroid Carcinoma | Anaplastic Thyroid | Rare | Genomic QC Panel | Long term |
| | Carcinoma | | | |
| Thyroid Carcinoma | Anaplastic Thyroid | Rare | Genomic QC Panel | Long term |
| | Carcinoma | | | |
| Thyroid Carcinoma | Papillary Thyroid Carcinoma | Rare | Genomic QC Panel | Short term |
| Thyroid Carcinoma | Papillary Thyroid Carcinoma | Rare | Genomic QC Panel | Long term |
| Renal Cell Carcinoma | Renal Medullary Carcinoma | Rare | Genomic QC Panel | Short term |
| Renal Cell Carcinoma | Renal Medullary Carcinoma | Rare | Genomic QC Panel | Long term |
| Wilms Tumor | | Rare | Genomic QC Panel | Long term |
| Undifferentiated Sarcoma | | Rare | Genomic QC Panel | Long term |
| Spindle Cell Sarcoma | | Rare | Genomic QC Panel | Long term |
| Wilms Tumor | | Rare | Genomic QC Panel | Short term |
| Wilms Tumor | | Rare | Genomic QC Panel | Short term |
| Rhabdomyosarcoma | Alveolar | Rare | Genomic QC Panel | Short term |
| | Rhabdomyosarcoma | | | |
| Wilms Tumor | | Rare | Genomic QC Panel | Short term |
| Ewing Sarcoma | | Rare | Genomic QC Panel | Long term |
| Epithelioid Sarcoma | | Rare | Genomic QC Panel | Long term |
| Wilms Tumor | | Rare | Genomic QC Panel | Short term |
| Ewing Sarcoma | | Rare | Genomic QC Panel | Long term |
| Ewing Sarcoma | | Rare | Genomic QC Panel | Long term |
| Angiosarcoma | | Rare | Genomic QC Panel | Long term |
| Wilms Tumor | | Rare | Genomic QC Panel | Short term |
| Rhabdomyosarcoma | Embryonal | Rare | Genomic QC Panel | Long term |
| | Rhabdomyosarcoma | | | |
| Neuroblastoma | | Rare | Genomic QC Panel | Long term |
| Wilms Tumor | | Rare | Genomic QC Panel | Short term |
| Rhabdomyosarcoma | Alveolar | Rare | RT-PCR | Long term |
| | Rhabdomyosarcoma | | | |
| Osteosarcoma | | Rare | Genomic QC Panel | Long term |
| Wilms Tumor | | Rare | Genomic QC Panel | Long term |
| Neuroblastoma | | Rare | Genomic QC Panel | Long term |
| Wilms Tumor | | Rare | Genomic QC Panel | Long term |
| Wilms Tumor | | Rare | Genomic QC Panel | Long term |
| Uterine Carcinosarcoma | | Rare | Genomic QC Panel | Long term |
| Head and Neck Squamous | | Common | Genomic QC Panel | TBD |
| Cell Carcinoma | | | | |
| Head and Neck Squamous | | Common | Genomic QC Panel | Long term |
| Cell Carcinoma | | | | |
| Head and Neck Squamous | | Common | Genomic QC Panel | Long term |
| Cell Carcinoma | | | | |
| Lung Carcinoma | | Common | Genomic QC Panel | TBD |
| Lung Carcinoma | | Common | Genomic QC Panel | TBD |
| Lung Carcinoma | | Common | Genomic QC Panel | Long term |
| Lung Carcinoma | | Common | Genomic QC Panel | TBD |
| Lung Carcinoma | | Common | Genomic QC Panel | TBD |
| Lung Carcinoma | Non Small Cell Lung Cancer | Common | Genomic QC Panel | Long term |
| Lung Carcinoma | Small Cell Lung Cancer | Common | Genomic QC Panel | Long term |
| Lung Carcinoma | Small Cell Lung Cancer | Common | Genomic QC Panel | Long term |
| Lung Carcinoma | Small Cell Lung Cancer | Common | Genomic QC Panel | Long term |
| Lung Carcinoma | Small Cell Lung Cancer | Common | Genomic QC Panel | Long term |
| Lung Carcinoma | Small Cell Lung Cancer | Common | Genomic QC Panel | Long term |
| Melanoma | | Common | Genomic QC Panel | TBD |
| Leiomyosarcoma | | Rare | Genomic QC Panel | TBD |
| Leiomyosarcoma | | Rare | Genomic QC Panel | TBD |
| Leiomyosarcoma | | Rare | Genomic QC Panel | Long term |
| Thyroid Carcinoma | | Rare | Genomic QC Panel | Long term |
| Uveal Melanoma | | Rare | Genomic QC Panel | TBD |
| Uveal Melanoma | | Rare | Genomic QC Panel | Long term |
| Uveal Melanoma | | Rare | Genomic QC Panel | TBD |
| Uveal Melanoma | | Rare | Genomic QC Panel | TBD |
| Lung Carcinoma | Small Cell Lung Cancer | Common | Genomic QC Panel | Long term |
| Lung Carcinoma | Small Cell Lung Cancer | Common | Genomic QC Panel | TBD |
| Lung Carcinoma | Small Cell Lung Cancer | Common | Genomic QC Panel | TBD |
| Leiomyosarcoma | | Rare | Genomic QC Panel | Short term |
| Liposarcoma | | Rare | Genomic QC Panel | TBD |
| Liposarcoma | | Rare | Genomic QC Panel | TBD |
| Liposarcoma | | Rare | Genomic QC Panel | TBD |
| Liposarcoma | | Rare | Genomic QC Panel | TBD |
| Liposarcoma | | Rare | Genomic QC Panel | TBD |
| Wilms Tumor | | Rare | Genomic QC Panel | Short term |
| Wilms Tumor | | Rare | Genomic QC Panel | TBD |
| Wilms Tumor | | Rare | Genomic QC Panel | TBD |
| Wilms Tumor | | Rare | Genomic QC Panel | TBD |
| Wilms Tumor | | Rare | Genomic QC Panel | TBD |
| Wilms Tumor | | Rare | Genomic QC Panel | TBD |
| Wilms Tumor | | Rare | Genomic QC Panel | TBD |
| Wilms Tumor | | Rare | Genomic QC Panel | Short term |
| Leiomyosarcoma | | Rare | Genomic QC Panel | Long term |
| Leiomyosarcoma | | Rare | Genomic QC Panel | TBD |
| Leiomyosarcoma | | Rare | Genomic QC Panel | TBD |
| Leiomyosarcoma | | Rare | Genomic QC Panel | TBD |
| Leiomyosarcoma | | Rare | Genomic QC Panel | Short term |
| Leiomyosarcoma | | Rare | Genomic QC Panel | TBD |
| Leiomyosarcoma | | Rare | Genomic QC Panel | Long term |
| Leiomyosarcoma | | Rare | Genomic QC Panel | Long term |
| Clear Cell Sarcoma | | Rare | Genomic QC Panel | TBD |
| Clear Cell Sarcoma | | Rare | Genomic QC Panel | Long term |
| Renal Cell Carcinoma | Clear Cell Renal Cell | Common | Genomic QC Panel | TBD |
| | Carcinoma | | | |
| Renal Cell Carcinoma | Papillary Renal Cell | Rare | Genomic QC Panel | TBD |
| | Carcinoma | | | |

| | | | Status in model | Patient tumor |
|---|---|---|---|---|
| Culture | Culture | Pass | derivation | tissue or PDX |
| system | media | Expansion? | pipeline | tumor tissue? |
| 3D organoid | BEGM, OPAC | Yes | Expansion Complete | Patient tumor |
| | | | | tissue |
| 3D organoid | AR5 | No attempt yet | Nursery Complete | Patient tumor |
| | | | | tissue |
| 3D organoid | CM, SMGM | No | Terminated | Patient tumor |
| | | | | tissue |
| 3D organoid | AR5, OPAC | Yes | Expansion Complete | Patient tumor |
| | | | | tissue |
| 2D adherent | SMGM | Yes | Expansion Complete | Patient tumor |
| | | | | tissue |
| 2D adherent | RETM | Yes | Expansion Complete | Patient tumor |
| | | | | tissue |
| 3D organoid | OPAC | In Progress | Expansion | Patient tumor |
| | | | | tissue |
| 3D organoid | AR5, SMGM | In Progress | Expansion | Patient tumor |
| | | | | tissue |
| 3D organoid | BCXJ, M87, SMGM | In Progress | Expansion | Patient tumor |
| | | | | tissue |
| 2D adherent | AR5 | Yes | Expansion Complete | Patient tumor |
| | | | | tissue |
| 2D adherent | SMGM | Yes | Expansion Complete | Patient tumor |
| | | | | tissue |
| 2D adherent | SMGM | Yes | Expansion Complete | Patient tumor |
| | | | | tissue |
| 2D adherent | AR5 | Yes | Expansion Complete | Patient tumor |
| | | | | tissue |
| 2D adherent | SMGM | Yes | Expansion Complete | Patient tumor |
| | | | | tissue |
| 2D adherent | CM | Yes | Expansion Complete | Patient tumor |
| | | | | tissue |
| 2D adherent | AR5 | Yes | Expansion Complete | Patient tumor |
| | | | | tissue |
| 2D adherent | CM | Yes | Expansion Complete | Patient tumor |
| | | | | tissue |
| 2D adherent | CM | Yes | Expansion Complete | Patient tumor |
| | | | | tissue |
| 2D adherent | SMGM | Yes | Expansion Complete | Patient tumor |
| | | | | tissue |
| 2D adherent | SMGM | In Progress | Expansion | Patient tumor |
| | | | | tissue |
| 2D adherent | SMGM | Yes | Expansion Complete | Patient tumor |
| | | | | tissue |
| 2D adherent | SMGM | Yes | Expansion Complete | Patient tumor |
| | | | | tissue |
| 2D adherent | AR5 | In Progress | Expansion | Patient tumor |
| | | | | tissue |
| 2D adherent | AR5 | Yes | Expansion Complete | Patient tumor |
| | | | | tissue |
| 2D adherent | RETM | Yes | Expansion Complete | Patient tumor |
| | | | | tissue |
| 2D adherent | SMGM | Yes | Expansion Complete | Patient tumor |
| | | | | tissue |
| 2D adherent | AR5 | Yes | Expansion Complete | Patient tumor |
| | | | | tissue |
| 2D adherent | SMGM | Yes | Expansion Complete | Patient tumor |
| | | | | tissue |
| 2D adherent | RETM | Yes | Expansion Complete | Patient tumor |
| | | | | tissue |
| 2D adherent | DMEM-10 | Yes | Expansion Complete | Patient tumor |
| | | | | tissue |
| 2D adherent | DMEM-10 | Yes | Expansion Complete | Patient tumor |
| | | | | tissue |
| 2D adherent | DMEM-10 | Yes | Expansion Complete | Patient tumor |
| | | | | tissue |
| 2D adherent | DMEM-10 | Yes | Expansion Complete | Patient tumor |
| | | | | tissue |
| 2D adherent | AR5 | Yes | Expansion Complete | Patient tumor |
| | | | | tissue |
| 2D adherent | AR5 | Yes | Expansion Complete | Patient tumor |
| | | | | tissue |
| 2D adherent | SMGM | In Progress | Expansion | Patient tumor |
| | | | | tissue |
| 2D adherent | RETM | Yes | Expansion Complete | Patient tumor |
| | | | | tissue |
| 2D adherent | CM | Yes | Expansion Complete | Patient tumor |
| | | | | tissue |
| 2D adherent | CM | No | Terminated | Patient tumor |
| | | | | tissue |
| 2D adherent | NSA | Yes | On Hold | Patient tumor |
| | | | | tissue |
| 2D adherent | SMGM | Yes | Expansion Complete | Patient tumor |
| | | | | tissue |
| 2D mix | AR5 | Yes | Expansion Complete | Patient tumor |
| | | | | tissue |
| 2D adherent | NSA | Yes | On Hold | Patient tumor |
| | | | | tissue |
| 2D adherent | CM | No | Terminated | Patient tumor |
| | | | | tissue |
| 2D adherent | SMGM | Yes | Expansion Complete | Patient tumor |
| | | | | tissue |
| 2D adherent | AR5 | Yes | Expansion Complete | Patient tumor |
| | | | | tissue |
| 2D adherent | RETM | Yes | Expansion Complete | Patient tumor |
| | | | | tissue |
| 2D mix | CM | Yes | Expansion Complete | Patient tumor |
| | | | | tissue |
| 2D adherent | CM | Yes | Expansion Complete | Patient tumor |
| | | | | tissue |
| 2D adherent | NSA | Yes | Expansion Complete | Patient tumor |
| | | | | tissue |
| 3D organoid | AR5, OPAC | No | Terminated | Patient tumor |
| | | | | tissue |
| 2D mix | AR5 | Yes | Expansion Complete | Patient tumor |
| | | | | tissue |
| 3D organoid | AR5, OPAC | Yes | Expansion Complete | Patient tumor |
| | | | | tissue |
| 2D adherent | AR5 | Yes | Expansion Complete | Patient tumor |
| | | | | tissue |
| 3D organoid | OPAC, RETM | No | Terminated | Patient tumor |
| | | | | tissue |
| 2D adherent | CM | Yes | Expansion Complete | Patient tumor |
| | | | | tissue |
| 3D organoid | OPAC | No | Terminated | Patient tumor |
| | | | | tissue |
| 3D organoid | OPAC, RETM | No | Terminated | Patient tumor |
| | | | | tissue |
| 3D organoid | AR5, SMGM | Yes | Expansion Complete | Patient tumor |
| | | | | tissue |
| 3D organoid | AR5, SMGM | In Progress | Expansion | Patient tumor |
| | | | | tissue |
| 3D organoid | SMGM | No | Terminated | Patient tumor |
| | | | | tissue |
| 3D organoid | AR5, RETM | No | Terminated | Patient tumor |
| | | | | tissue |
| 2D adherent | AR5 | Yes | Expansion Complete | Patient tumor |
| | | | | tissue |
| 2D adherent | SMGM | Yes | Expansion Complete | Patient tumor |
| | | | | tissue |
| 3D organoid | OPAC, RETM | No | Terminated | Patient tumor |
| | | | | tissue |
| 2D suspension | EGM2, OPAC | Yes | Expansion Complete | Patient tumor |
| | | | | tissue |
| 3D organoid | OPAC, RETM | In Progress | Expansion | Patient tumor |
| | | | | tissue |
| 3D organoid | AR5, OPAC | No | Terminated | Patient tumor |
| | | | | tissue |
| 2D suspension | NSA | No | Terminated | Patient tumor |
| | | | | tissue |
| 2D adherent | AR5 | Yes | Expansion Complete | Patient tumor |
| | | | | tissue |
| 2D adherent | RETM | Yes | Expansion Complete | Patient tumor |
| | | | | tissue |
| 2D adherent | AR5 | Yes | Expansion Complete | Patient tumor |
| | | | | tissue |
| 2D adherent | RETM | Yes | Expansion Complete | Patient tumor |
| | | | | tissue |
| 2D adherent | CM | Yes | Expansion Complete | Patient tumor |
| | | | | tissue |
| 2D adherent | SMGM | Yes | Expansion Complete | Patient tumor |
| | | | | tissue |
| 2D adherent | SMGM | Yes | Expansion Complete | Patient tumor |
| | | | | tissue |
| 2D adherent | CM | Yes | Expansion Complete | Patient tumor |
| | | | | tissue |
| 2D adherent | OGM | Yes | Expansion Complete | Patient tumor |
| | | | | tissue |
| 2D adherent | HT Media Screen | No | Terminated | Patient tumor |
| | | | | tissue |
| 2D adherent | CM | Yes | Expansion Complete | Patient tumor |
| | | | | tissue |
| 2D adherent | CM, RETM | Yes | Expansion Complete | Patient tumor |
| | | | | tissue |
| 2D adherent | CM, RETM | No | Terminated | Patient tumor |
| | | | | tissue |
| 2D adherent | AR5, CM | Yes | Expansion Complete | Patient tumor |
| | | | | tissue |
| 2D adherent | M87, RETM | No | Terminated | Patient tumor |
| | | | | tissue |
| 2D adherent | DMEM | Yes | Expansion Complete | Patient tumor |
| | | | | tissue |
| 2D adherent | AR5, RETM | Yes | Expansion Complete | Patient tumor |
| | | | | tissue |
| 2D adherent | AR5, M87 | Yes | Expansion Complete | Patient tumor |
| | | | | tissue |
| 3D organoid | AR5, EGM2 | Yes | Expansion Complete | Patient tumor |
| | | | | tissue |
| 2D adherent | AR5, RETM | No | Terminated | Patient tumor |
| | | | | tissue |
| 3D organoid | OPAC, RETM | Yes | Expansion Complete | Patient tumor |
| | | | | tissue |
| 2D suspension | AR5 | In Progress | Expansion | Patient tumor |
| | | | | tissue |
| 2D adherent | AR5 | Yes | Expansion Complete | Patient tumor |
| | | | | tissue |
| 2D adherent | CM | Yes | Expansion Complete | Patient tumor |
| | | | | tissue |
| 2D adherent | AR5, SMGM | Yes | Expansion Complete | Patient tumor |
| | | | | tissue |
| 2D adherent | AR5, SMGM | Yes | Expansion Complete | Patient tumor |
| | | | | tissue |
| 3D organoid | BEGM, SMGM | No | Terminated | Patient tumor |
| | | | | tissue |
| 3D organoid | EGM2, SMGM | In Progress | Expansion | Patient tumor |
| | | | | tissue |
| 3D organoid | AR5, SMGM | Yes | Expansion Complete | Patient tumor |
| | | | | tissue |
| 3D organoid | EGM2, SMGM | Yes | Expansion Complete | Patient tumor |
| | | | | tissue |
| 3D organoid | BEGM, SMGM | In Progress | Expansion | Patient tumor |
| | | | | tissue |
| 3D organoid | BEGM, SMGM | No | Terminated | Patient tumor |
| | | | | tissue |
| 3D organoid | AR5, CM | No | Terminated | Patient tumor |
| | | | | tissue |
| 3D organoid | CM, SMGM | In Progress | Expansion | Patient tumor |
| | | | | tissue |
| 3D organoid | AR5, CM | No | Terminated | Patient tumor |
| | | | | tissue |
| 2D adherent | RETM | Yes | Expansion Complete | Patient tumor |
| | | | | tissue |
| 2D adherent | RETM | Yes | Expansion Complete | Patient tumor |
| | | | | tissue |
| 2D adherent | SMGM | Yes | Expansion Complete | Patient tumor |
| | | | | tissue |
| 2D adherent | SMGM | Yes | Expansion Complete | Patient tumor |
| | | | | tissue |
| 2D adherent | RETM | Yes | Expansion Complete | Patient tumor |
| | | | | tissue |
| 2D adherent | SMGM | Yes | Expansion Complete | Patient tumor |
| | | | | tissue |
| 2D adherent | SMGM | Yes | Expansion Complete | Patient tumor |
| | | | | tissue |
| 2D adherent | SMGM | Yes | Expansion Complete | Patient tumor |
| | | | | tissue |
| 2D adherent | SMGM | Yes | Expansion Complete | Patient tumor |
| | | | | tissue |
| 2D adherent | AR5 | Yes | Expansion Complete | Patient tumor |
| | | | | tissue |
| 2D adherent | RETM | Yes | Expansion Complete | Patient tumor |
| | | | | tissue |
| 2D suspension | XVIVO | Yes | Expansion Complete | PDX |
| 2D suspension | XVIVO | Yes | Expansion Complete | PDX |
| 2D suspension | AR5 | Yes | Expansion Complete | PDX |
| 2D suspension | XVIVO | Yes | Expansion Complete | Patient tumor |
| | | | | tissue |
| 2D suspension | XVIVO | Yes | Expansion Complete | Patient tumor |
| | | | | tissue |
| 2D suspension | XVIVO | No attempt yet | On Hold | PDX |
| 2D adherent | M87 | Yes | Expansion Complete | Patient tumor |
| | | | | tissue |
| 2D adherent | RETM | Yes | Expansion Complete | Patient tumor |
| | | | | tissue |
| 2D adherent | RETM | No attempt yet | On Hold | Patient tumor |
| | | | | tissue |
| 2D adherent | SMGM | No attempt yet | On Hold | PDX |
| 2D adherent | CM | Yes | Expansion Complete | PDX |
| 2D adherent | AR5, RETM | No attempt yet | On Hold | PDX |
| 2D adherent | RETM | Yes | Expansion Complete | PDX |
| 2D adherent | RETM | On Hold | On Hold | PDX |
| 2D adherent | CM | Yes | Expansion Complete | Patient tumor |
| | | | | tissue |
| 2D adherent | CM | Yes | Expansion Complete | Patient tumor |
| | | | | tissue |
| 2D adherent | CM | Yes | Expansion Complete | Patient tumor |
| | | | | tissue |
| 2D adherent | CM | Yes | Expansion Complete | Patient tumor |
| | | | | tissue |
| 2D adherent | CM | Yes | Expansion Complete | Patient tumor |
| | | | | tissue |
| 2D adherent | RPMI-10 | Yes | Expansion Complete | Patient tumor |
| | | | | tissue |
| 2D adherent | CM | No | Terminated | Patient tumor |
| | | | | tissue |
| 2D adherent | CM | Yes | Expansion Complete | Patient tumor |
| | | | | tissue |
| 2D adherent | CM | No | Terminated | Patient tumor |
| | | | | tissue |
| 2D mix | CM | Yes | Expansion Complete | Patient tumor |
| | | | | tissue |
| 2D adherent | CM | Yes | Expansion Complete | Patient tumor |
| | | | | tissue |
| 2D adherent | RPMI-10 | Yes | Expansion Complete | Patient tumor |
| | | | | tissue |
| 2D mix | CM | Yes | Expansion Complete | Patient tumor |
| | | | | tissue |
| 2D adherent | CM | No | Terminated | Patient tumor |
| | | | | tissue |
| 2D adherent | CM | No | Terminated | Patient tumor |
| | | | | tissue |
| 2D adherent | CM | No | Terminated | Patient tumor |
| | | | | tissue |
| 2D adherent | CM | No | Terminated | Patient tumor |
| | | | | tissue |
| 2D mix | SMGM | Yes | Expansion Complete | Patient tumor |
| | | | | tissue |
| 2D adherent | SMGM | Yes | Expansion Complete | Patient tumor |
| | | | | tissue |
| 2D adherent | CM | No | Terminated | Patient tumor |
| | | | | tissue |
| 2D mix | CM | Yes | Expansion Complete | Patient tumor |
| | | | | tissue |
| 2D adherent | CM | Yes | Expansion Complete | Patient tumor |
| | | | | tissue |
| 2D adherent | SMGM | Yes | Expansion Complete | Patient tumor |
| | | | | tissue |
| 2D adherent | CM | No | Terminated | PDX |
| 2D adherent | CM | Yes | Expansion Complete | Patient tumor |
| | | | | tissue |
| 2D adherent | RETM | Yes | Expansion Complete | Patient tumor |
| | | | | tissue |
| 2D adherent | CM | No | Terminated | Patient tumor |
| | | | | tissue |
| 2D mix | CM | Yes | Expansion Complete | Patient tumor |
| | | | | tissue |
| 2D adherent | SMGM | Yes | Expansion Complete | Patient tumor |
| | | | | tissue |
| 2D adherent | CM | Yes | Expansion Complete | Patient tumor |
| | | | | tissue |
| 2D adherent | CM | Yes | Expansion Complete | Patient tumor |
| | | | | tissue |
| 2D mix | CM | Yes | Expansion Complete | Patient tumor |
| | | | | tissue |
| 2D adherent | CM | Yes | Expansion Complete | Patient tumor |
| | | | | tissue |
| 2D suspension | WiT-P | Yes | Expansion Complete | Patient tumor |
| | | | | tissue |
| 3D organoid | OPAC | No attempt yet | Nursery Complete | Patient tumor |
| | | | | tissue |
| 3D organoid | CM | Yes | Expansion Complete | Patient tumor |
| | | | | tissue |
| 3D organoid | OPAC | Yes | Expansion Complete | Patient tumor |
| | | | | tissue |
| 3D organoid | AR5/M26/C5a/BMP9 | In Progress | Expansion | Patient tumor |
| | | | | tissue |
| 3D organoid | RETM/M26/C5a/BMP9 | No attempt yet | Nursery Complete | Patient tumor |
| | | | | tissue |
| 3D organoid | AR5/MBM | Yes | Expansion Complete | Patient tumor |
| | | | | tissue |
| 3D organoid | OPAC/MBM | No attempt yet | Nursery Complete | Patient tumor |
| | | | | tissue |
| 3D organoid | OPAC/MBM | No attempt yet | Nursery Complete | Patient tumor |
| | | | | tissue |
| 3D organoid | AR5/M26/C5a/BMP9 | Yes | Expansion Complete | Patient tumor |
| | | | | tissue |
| 3D organoid | AR5/OPAC | Yes | Expansion Complete | Patient tumor |
| | | | | tissue |
| 3D organoid | AR5/OPAC/C5a/BMP9/Nutlin | Yes | Expansion Complete | Patient tumor |
| | | | | tissue |
| 3D organoid | OPAC/C5a/BMP9/Nutlin | Yes | Expansion Complete | Patient tumor |
| | | | | tissue |
| 3D organoid | OPAC/MBM | Yes | Expansion Complete | Patient tumor |
| | | | | tissue |
| 3D organoid | OPAC/MBM/C5a/BMP9/Nutlin | Yes | Expansion Complete | Patient tumor |
| | | | | tissue |
| 2D adherent | AR5 | No attempt yet | Nursery Complete | Patient tumor |
| | | | | tissue |
| 2D adherent | BEGM/AR5 | No attempt yet | Nursery Complete | Patient tumor |
| | | | | tissue |
| 2D adherent | CM/BEGM | No attempt yet | Nursery Complete | Patient tumor |
| | | | | tissue |
| 2D adherent | RETM/SMGM | Yes | Expansion Complete | Patient tumor |
| | | | | tissue |
| 2D adherent | RETM | Yes | Expansion Complete | Patient tumor |
| | | | | tissue |
| 2D adherent | AR5/RETM/B27 | No attempt yet | Nursery Complete | Patient tumor |
| | | | | tissue |
| 2D adherent | CM/RETM/B27 | Yes | Expansion Complete | Patient tumor |
| | | | | tissue |
| 2D adherent | RETM/SMGM/B27 | No attempt yet | Nursery Complete | Patient tumor |
| | | | | tissue |
| 2D adherent | SMGM/B27 | No attempt yet | Nursery Complete | Patient tumor |
| | | | | tissue |
| 3D organoid | AR5/M26/C5a/BMP9 | Yes | Expansion Complete | Patient tumor |
| | | | | tissue |
| 3D organoid | OPAC/M26/C5a/BMP9 | No attempt yet | Nursery Complete | Patient tumor |
| | | | | tissue |
| 3D organoid | RETM/M26/C5a/BMP9 | No attempt yet | Nursery Complete | Patient tumor |
| | | | | tissue |
| 2D adherent | BEGM/CM | No | Terminated | Patient tumor |
| | | | | tissue |
| 2D adherent | RETM | No attempt yet | Nursery Complete | Patient tumor |
| | | | | tissue |
| 2D adherent | RETM/AR5 | No attempt yet | Nursery Complete | Patient tumor |
| | | | | tissue |
| 2D adherent | RETM/E8 | No attempt yet | Nursery Complete | Patient tumor |
| | | | | tissue |
| 2D adherent | RETM/SMGM | No attempt yet | Nursery Complete | Patient tumor |
| | | | | tissue |
| 2D adherent | SMGM/E8 | No attempt yet | Nursery Complete | Patient tumor |
| | | | | tissue |
| 3D organoid | AR5 | No | Terminated | Patient tumor |
| | | | | tissue |
| 3D organoid | BEGM/AR5 | No attempt yet | Nursery Complete | Patient tumor |
| | | | | tissue |
| 3D organoid | CM | No attempt yet | Nursery Complete | Patient tumor |
| | | | | tissue |
| 3D organoid | CM/AR5 | No attempt yet | Nursery Complete | Patient tumor |
| | | | | tissue |
| 3D organoid | CM/BEGM | No attempt yet | Nursery Complete | Patient tumor |
| | | | | tissue |
| 3D organoid | CM/SMGM | No attempt yet | Nursery Complete | Patient tumor |
| | | | | tissue |
| 3D organoid | SMGM | In Progress | Expansion | Patient tumor |
| | | | | tissue |
| 3D organoid | SMGM/AR5 | No | Terminated | Patient tumor |
| | | | | tissue |
| 2D mix | Des23CM/EGM | Yes | Expansion Complete | Patient tumor |
| | | | | tissue |
| 2D mix | EGM/E8 | No attempt yet | Nursery Complete | Patient tumor |
| | | | | tissue |
| 2D mix | SMGM/E8 | No attempt yet | Nursery Complete | Patient tumor |
| | | | | tissue |
| 2D adherent | EGM/E8 | In Progress | Expansion | Patient tumor |
| | | | | tissue |
| 2D adherent | AR5/E8 | No | Terminated | Patient tumor |
| | | | | tissue |
| 2D adherent | SMGM/AR5 | No attempt yet | Nursery Complete | Patient tumor |
| | | | | tissue |
| 2D adherent | CM/RETM | Yes | Expansion Complete | Patient tumor |
| | | | | tissue |
| 2D adherent | M87/RETM | Yes | Expansion Complete | Patient tumor |
| | | | | tissue |
| 2D adherent | M87/E8 | No attempt yet | Nursery Complete | Patient tumor |
| | | | | tissue |
| 2D adherent | RETM/E8 | Yes | Expansion Complete | Patient tumor |
| | | | | tissue |
| 3D organoid | SMGM/AR5 | In Progress | Expansion | Patient tumor |
| | | | | tissue |
| 3D organoid | SMGM/AR5 | In Progress | Expansion | Patient tumor |
| | | | | tissue |

| HCMI | Deposited at | | Patient tumor | |
|---|---|---|---|---|
| model? | ATCC? | Accessibility | type | Gender |
| No | N/A | Currently at CCLF | Metastatic | |
| TBD | TBD | Currently at CCLF | Metastatic | |
| N/A | N/A | Currently at CCLF | Primary | Male |
| Yes | Yes | Deposited at ATCC; not yet | Primary | Male |
| | | available online | | |
| Yes | Yes | Available at ATCC | Metastatic | Female |
| Yes | Yes | Deposited at ATCC; not yet | Metastatic | Male |
| | | available online | | |
| TBD | TBD | Currently at CCLF | Primary | Male |
| TBD | TBD | Currently at CCLF | Primary | Male |
| TBD | TBD | Currently at CCLF | Metastatic | Female |
| Yes | Yes | Available at ATCC | Metastatic | Male |
| Yes | Yes | Available at ATCC | Metastatic | Female |
| Yes | Yes | Deposited at ATCC; not yet | Metastatic | Female |
| | | available online | | |
| Yes | Yes | Deposited at ATCC; not yet | Unknown | Female |
| | | available online | | |
| TBD | TBD | Currently at CCLF | Secondary | Female |
| Yes | Yes | Deposited at ATCC; not yet | Metastatic | Male |
| | | available online | | |
| TBD | TBD | Currently at CCLF | Metastatic | Male |
| TBD | TBD | Currently at CCLF | Metastatic | Male |
| Yes | Yes | Available at ATCC | Metastatic | Male |
| Yes | Yes | Available at ATCC | Unknown | Female |
| TBD | TBD | Currently at CCLF | Metastatic | Female |
| Yes | Yes | Deposited at ATCC; not yet | Metastatic | Female |
| | | available online | | |
| Yes | Yes | Deposited at ATCC; not yet | Metastatic | Male |
| | | available online | | |
| TBD | TBD | Currently at CCLF | Metastatic | Male |
| Yes | Yes | Deposited at ATCC; not yet | Metastatic | Male |
| | | available online | | |
| Yes | Yes | Deposited at ATCC; not yet | Metastatic | Male |
| | | available online | | |
| Yes | Yes | Deposited at ATCC; not yet | Metastatic | Male |
| | | available online | | |
| Yes | Yes | Deposited at ATCC; not yet | Metastatic | Male |
| | | available online | | |
| Yes | Yes | Deposited at ATCC; not yet | Metastatic | Female |
| | | available online | | |
| Yes | Yes | Deposited at ATCC; not yet | Metastatic | Male |
| | | available online | | |
| No | N/A | Currently at CCLF | Metastatic | Male |
| Yes | Yes | Deposited at ATCC; not yet | Metastatic | Female |
| | | available online | | |
| No | N/A | Currently at CCLF | Unknown | Unknown |
| Yes | Yes | Deposited at ATCC; not yet | Metastatic | Male |
| | | available online | | |
| Yes | Yes | Deposited at ATCC; not yet | Metastatic | Female |
| | | available online | | |
| Yes | Yes | Deposited at ATCC; not yet | Metastatic | Male |
| | | available online | | |
| TBD | TBD | Currently at CCLF | Primary | Male |
| Yes | Yes | Deposited at ATCC; not yet | Metastatic | Female |
| | | available online | | |
| Yes | Yes | Available at ATCC | Metastatic | Male |
| N/A | N/A | Currently at CCLF | Metastatic | Female |
| TBD | TBD | Currently at CCLF | Primary | Male |
| Yes | Yes | Available at ATCC | Metastatic | Male |
| Yes | Yes | Available at ATCC | Metastatic | Male |
| TBD | TBD | Currently at CCLF | Primary | Male |
| N/A | N/A | Currently at CCLF | Metastatic | Unknown |
| No | N/A | Currently at CCLF | Metastatic | Male |
| No | N/A | Currently at CCLF | Metastatic | Female |
| No | N/A | Currently at CCLF | Metastatic | Female |
| Yes | Yes | Available at ATCC | Metastatic | Male |
| No | N/A | Currently at CCLF | Primary | Male |
| No | N/A | Currently at CCLF | Primary | Female |
| N/A | N/A | Currently at CCLF | Metastatic | Female |
| Yes | Yes | Deposited at ATCC; not yet | Metastatic | Female |
| | | available online | | |
| Yes | Yes | Deposited at ATCC; not yet | Metastatic | Male |
| | | available online | | |
| Yes | Yes | Deposited at ATCC; not yet | Metastatic | Female |
| | | available online | | |
| N/A | N/A | Currently at CCLF | Metastatic | Female |
| Yes | Yes | Deposited at ATCC; not yet | Metastatic | Male |
| | | available online | | |
| N/A | N/A | Currently at CCLF | Metastatic | Female |
| N/A | N/A | Currently at CCLF | Metastatic | Female |
| Pending | Pending | Currently at CCLF | Metastatic | Female |
| TBD | TBD | Currently at CCLF | Metastatic | Female |
| N/A | N/A | Currently at CCLF | Metastatic | Female |
| N/A | N/A | Currently at CCLF | Metastatic | Female |
| Yes | Yes | Deposited at ATCC; not yet | Metastatic | Female |
| | | available online | | |
| Yes | Yes | Deposited at ATCC; not yet | Metastatic | Female |
| | | available online | | |
| N/A | N/A | Currently at CCLF | Metastatic | Female |
| Yes | Yes | Deposited at ATCC; not yet | Primary | Male |
| | | available online | | |
| TBD | TBD | Currently at CCLF | Metastatic | Female |
| No | N/A | Currently at CCLF | Metastatic | Female |
| N/A | N/A | Currently at CCLF | Recurrent | Female |
| Yes | Yes | Deposited at ATCC; not yet | Metastatic | Female |
| | | available online | | |
| Yes | Yes | Deposited at ATCC; not yet | Metastatic | Female |
| | | available online | | |
| No | N/A | Currently at CCLF | Metastatic | Female |
| No | N/A | Currently at CCLF | Primary | Female |
| Yes | Yes | Available at ATCC | Metastatic | Unknown |
| Yes | Yes | Available at ATCC | Metastatic | Male |
| Yes | Yes | Available at ATCC | Metastatic | Male |
| Yes | Yes | Deposited at ATCC; not yet | Primary | Female |
| | | available online | | |
| Yes | Yes | Available at ATCC | Primary | Female |
| N/A | N/A | Currently at CCLF | Primary | Female |
| Yes | Yes | Deposited at ATCC; not yet | Primary | Male |
| | | available online | | |
| Yes | Yes | Deposited at ATCC; not yet | Primary | Male |
| | | available online | | |
| N/A | N/A | Currently at CCLF | Primary | Male |
| Yes | Pending | Currently at CCLF | Primary | Female |
| No | N/A | Currently at CCLF | Primary | Male |
| Yes | Pending | Currently at CCLF | Metastatic | Female |
| Yes | Pending | Currently at CCLF | Primary | Female |
| Yes | Yes | Deposited at ATCC; not yet | Primary | Female |
| | | available online | | |
| Yes | Yes | Deposited at ATCC; not yet | Metastatic | Female |
| | | available online | | |
| N/A | N/A | Currently at CCLF | Primary | Female |
| Yes | Yes | Deposited at ATCC; not yet | Metastatic | Female |
| | | available online | | |
| TBD | TBD | Currently at CCLF | Metastatic | Female |
| Yes | Yes | Deposited at ATCC; not yet | Metastatic | Male |
| | | available online | | |
| Yes | Yes | Deposited at ATCC; not yet | Metastatic | Male |
| | | available online | | |
| No | N/A | Currently at CCLF | Primary | |
| Yes | Yes | Deposited at ATCC; not yet | Primary | Male |
| | | available online | | |
| N/A | N/A | Currently at CCLF | Primary | Female |
| TBD | TBD | Currently at CCLF | Primary | Male |
| Yes | Yes | Deposited at ATCC; not yet | Primary | Female |
| | | available online | | |
| Yes | Yes | Deposited at ATCC; not yet | Primary | Male |
| | | available online | | |
| TBD | TBD | Currently at CCLF | Primary | Male |
| N/A | N/A | Currently at CCLF | Primary | Female |
| N/A | N/A | Currently at CCLF | Primary | Male |
| Pending | Pending | Currently at CCLF | Primary | Male |
| N/A | N/A | Currently at CCLF | Primary | Male |
| Yes | Yes | Available at ATCC | Metastatic | |
| Yes | Yes | Deposited at ATCC; not yet | Metastatic | |
| | | available online | | |
| No | N/A | Currently at CCLF | Metastatic | |
| Yes | Yes | Deposited at ATCC; not yet | Primary | |
| | | available online | | |
| Yes | Yes | Available at ATCC | Metastatic | |
| Yes | Yes | Available at ATCC | Metastatic | |
| No | N/A | Currently at CCLF | Metastatic | |
| Yes | Yes | Available at ATCC | Metastatic | |
| Yes | Yes | Available at ATCC | Metastatic | |
| Yes | Yes | Available at ATCC | Metastatic | |
| Yes | Yes | Available at ATCC | Metastatic | |
| Yes | N/A | Currently at CCLF | Primary | |
| Yes | N/A | Currently at CCLF | Primary | |
| Yes | N/A | Currently at CCLF | Recurrent | |
| Yes | N/A | Currently at CCLF | Primary | |
| Yes | N/A | Currently at CCLF | Primary | |
| No | N/A | Currently at CCLF | Primary | |
| No | N/A | Currently at CCLF | Metastatic | |
| No | N/A | Currently at CCLF | Metastatic | |
| TBD | TBD | Currently at CCLF | Primary | |
| TBD | TBD | Currently at CCLF | Primary | |
| No | N/A | Currently at CCLF | Primary | |
| TBD | TBD | Currently at CCLF | | |
| No | N/A | Currently at CCLF | Primary | Male |
| TBD | TBD | Currently at CCLF | Primary | Male |
| No | N/A | Currently at CCLF | Metastatic | |
| No | N/A | Currently at CCLF | Primary | |
| No | N/A | Currently at CCLF | Metastatic | |
| No | N/A | Currently at CCLF | Primary | |
| No | N/A | Currently at CCLF | Metastatic | |
| Yes | N/A | Currently at CCLF | Metastatic | |
| N/A | N/A | Currently at CCLF | Primary | |
| No | N/A | Currently at CCLF | Metastatic | |
| N/A | N/A | Currently at CCLF | Primary | Male |
| Pending | Pending | Currently at CCLF | Primary | Male |
| Pending | Pending | Currently at CCLF | Primary | Female |
| Yes | Yes | Available at ATCC | Metastatic | Male |
| Yes | Yes | Available at ATCC | Primary | Female |
| No | N/A | Currently at CCLF | Primary | |
| No | N/A | Currently at CCLF | Primary | |
| No | N/A | Currently at CCLF | Primary | |
| N/A | N/A | Currently at CCLF | Primary | Unknown |
| Yes | Yes | Available at ATCC | Metastatic | Male |
| Yes | Yes | Available at ATCC | Metastatic | Female |
| No | N/A | Currently at CCLF | Primary | |
| Yes | Yes | Available at ATCC | Metastatic | Male |
| Yes | Yes | Available at ATCC | Metastatic | Male |
| No | N/A | Currently at CCLF | Primary | |
| N/A | N/A | Currently at CCLF | Primary | |
| Yes | Yes | Deposited at ATCC; not yet | Primary | Female |
| | | available online | | |
| Yes | Yes | Deposited at ATCC; not yet | Primary | Male |
| | | available online | | |
| No | N/A | Currently at CCLF | Primary | |
| Yes | Yes | Available at ATCC | Metastatic | Male |
| Yes | Yes | Available at ATCC | Primary | Male |
| Yes | Yes | Deposited at ATCC; not yet | Primary | Female |
| | | available online | | |
| No | N/A | Currently at CCLF | Metastatic | Male |
| Yes | Yes | Deposited at ATCC; not yet | Primary | Male |
| | | available online | | |
| Yes | Yes | Deposited at ATCC; not yet | Primary | Female |
| | | available online | | |
| Yes | N/A | Currently at CCLF | Primary | Female |
| TBD | TBD | Currently at CCLF | Primary | |
| Yes | N/A | Currently at CCLF | Primary | Unknown |
| Yes | N/A | Currently at CCLF | Primary | Unknown |
| TBD | TBD | Currently at CCLF | Primary | Female |
| TBD | TBD | Currently at CCLF | Primary | Female |
| Yes | Pending | Currently at CCLF | Primary | Female |
| No | N/A | Currently at CCLF | Primary | Female |
| No | N/A | Currently at CCLF | Primary | Female |
| Yes | Pending | Currently at CCLF | Metastatic | Female |
| Yes | Pending | Currently at CCLF | Metastatic | Female |
| No | N/A | Currently at CCLF | Metastatic | Female |
| No | N/A | Currently at CCLF | Metastatic | Female |
| No | N/A | Currently at CCLF | Metastatic | Female |
| No | N/A | Currently at CCLF | Metastatic | Female |
| No | N/A | Currently at CCLF | Metastatic | Female |
| No | N/A | Currently at CCLF | Metastatic | Female |
| No | N/A | Currently at CCLF | Metastatic | Female |
| Yes | Pending | Currently at CCLF | Metastatic | Female |
| Yes | Pending | Currently at CCLF | Metastatic | Unknown |
| No | N/A | Currently at CCLF | Unknown | Unknown |
| Yes | Pending | Currently at CCLF | Unknown | Unknown |
| No | N/A | Currently at CCLF | Unknown | Unknown |
| No | N/A | Currently at CCLF | Unknown | Unknown |
| Yes | Pending | Currently at CCLF | Metastatic | Male |
| No | N/A | Currently at CCLF | Metastatic | Male |
| No | N/A | Currently at CCLF | Metastatic | Male |
| No | N/A | Currently at CCLF | Primary | Male |
| TBD | TBD | Currently at CCLF | Recurrent | Male |
| TBD | TBD | Currently at CCLF | Recurrent | Male |
| TBD | TBD | Currently at CCLF | Recurrent | Male |
| TBD | TBD | Currently at CCLF | Recurrent | Male |
| TBD | TBD | Currently at CCLF | Recurrent | Male |
| No | N/A | Currently at CCLF | Primary | Female |
| TBD | TBD | Currently at CCLF | Primary | Female |
| TBD | TBD | Currently at CCLF | Primary | Female |
| TBD | TBD | Currently at CCLF | Primary | Female |
| TBD | TBD | Currently at CCLF | Primary | Female |
| TBD | TBD | Currently at CCLF | Primary | Female |
| TBD | TBD | Currently at CCLF | Primary | Female |
| No | N/A | Currently at CCLF | Primary | Female |
| TBD | TBD | Currently at CCLF | Metastatic | Female |
| No | N/A | Currently at CCLF | Metastatic | Female |
| No | N/A | Currently at CCLF | Metastatic | Female |
| TBD | TBD | Currently at CCLF | Primary | Unknown |
| No | N/A | Currently at CCLF | Primary | Unknown |
| TBD | TBD | Currently at CCLF | Primary | Unknown |
| Yes | TBD | Currently at CCLF | Metastatic | Unknown |
| Yes | Yes | Deposited at ATCC; not yet | Metastatic | Female |
| | | available online | | |
| No | N/A | Currently at CCLF | Unknown | Female |
| Yes | Pending | Currently at CCLF | Unknown | Female |
| TBD | TBD | Currently at CCLF | Primary | Male |
| TBD | TBD | Currently at CCLF | Primary | Male |

| Age at | Race or | Treatment | Status in | Quarter model |
|---|---|---|---|---|
| Collection | Ethnicity | History | DepMap | added to this list |
| | | | N/A | Q2 2018 |
| | | | N/A | Q1 2019 |
| 49 | Black or African American | Treatment Naive | N/A | Q1 2019 |
| 64 | Black or African American | Treatment Naive | N/A | Q2 2019 |
| 56 | White | Previously received | N/A | Q2 2019 |
| | | treatment | | |
| 71 | White | Currently undergoing active | N/A | Q3 2019 |
| | | treatments | | |
| 52 | White | Unknown | N/A | Q1 2021 |
| 52 | White | Unknown | N/A | Q1 2021 |
| 72 | White | Previously received | N/A | Q4 2019 |
| | | treatment | | |
| 65 | White | Currently undergoing active | N/A | Q2 2019 |
| | | treatments | | |
| 79 | Unknown | Unknown | N/A | Q2 2019 |
| 59 | White | Previously Received | N/A | Q1 2019 |
| | | Treatment | | |
| 84 | Unknown | Unknown | N/A | Q2 2019 |
| 64 | White | Unknown | N/A | Q2 2019 |
| 79 | White | Currently undergoing active | N/A | Q3 2019 |
| | | treatments | | |
| 79 | White | Currently undergoing active | N/A | Q1 2021 |
| | | treatment | | |
| 79 | White | Currently undergoing active | N/A | Q1 2021 |
| | | treatment | | |
| 55 | White | Unknown | N/A | Q3 2019 |
| 69 | White | Unknown | N/A | Q4 2019 |
| 46 | White | Treatment Naive | N/A | Q1 2021 |
| 48 | White | Unknown | N/A | Q4 2019 |
| 69 | White | Treatment Naive | N/A | Q1 2020 |
| 50 | White | Previously received | N/A | Q1 2021 |
| | | treatment | | |
| 67 | White | Previously Received | N/A | Q1 2020 |
| | | Treatment | | |
| 75 | White | Unknown | N/A | Q4 2019 |
| 67 | White | Unknown | N/A | Q1 2020 |
| 58 | White | Unknown | N/A | Q1 2020 |
| 64 | White | Unknown | N/A | Q1 2020 |
| 77 | White | Unknown | N/A | Q1 2020 |
| Unknown | Unknown | Unknown | N/A | Q1 2020 |
| Unknown | Unknown | Unknown | N/A | Q1 2020 |
| Unknown | Unknown | Unknown | N/A | Q1 2020 |
| Unknown | Unknown | Previously Received | N/A | Q1 2020 |
| | | Treatment | | |
| 80 | White | Unknown | N/A | Q1 2021 |
| 50 | White | Unknown | N/A | Q1 2021 |
| 67 | White | Unknown | N/A | Q1 2021 |
| 60 | White | Previously received | N/A | Q1 2021 |
| | | treatment | | |
| 79 | White | | N/A | Q2 2017 |
| 40 | White | | N/A | Q4 2017 |
| 61 | White | Treatment Naive | N/A | Q4 2017 |
| 75 | White | Currently undergoing active | N/A | Q1 2018 |
| | | treatments | | |
| 49 | White | Currently undergoing active | N/A | Q1 2018 |
| | | treatments | | |
| 6 | Unknown | Unknown | N/A | Q1 2018 |
| 83 | White | Previously received | N/A | Q1 2018 |
| | | treatment | | |
| 38 | White | Previously Received | N/A | Q4 2018 |
| | | Treatment | | |
| 74 | White | Previously Received | N/A | Q3 2018 |
| | | Treatment | | |
| 74 | White | Previously Received | N/A | Q3 2018 |
| | | Treatment | | |
| 73 | White | Previously Received | N/A | Q3 2018 |
| | | Treatment | | |
| 61 | White | Treatment Naive | N/A | Q4 2018 |
| 57 | White | Treatment Naive | N/A | Q3 2018 |
| 73 | Black or African American | Previously Received | N/A | Q3 2018 |
| | | Treatment | | |
| 67 | White | Previously Received | N/A | Q1 2019 |
| | | Treatment | | |
| 66 | White | Unknown | N/A | Q2 2019 |
| 49 | White | Treatment Naive | N/A | Q1 2021 |
| 72 | White | Currently undergoing active | N/A | Q2 2019 |
| | | treatments | | |
| 32 | White | Unknown | N/A | Q2 2019 |
| 62 | White | Previously Received | N/A | Q1 2020 |
| | | Treatment | | |
| 62 | White | Previously Received | N/A | Q1 2020 |
| | | Treatment | | |
| 66 | White | Previously Received | N/A | Q3 2019 |
| | | Treatment | | |
| 66 | White | Previously received | N/A | Q1 2021 |
| | | treatment | | |
| 75 | White | Treatment Naive | N/A | Q2 2019 |
| 62 | White | Currently undergoing active | N/A | Q2 2019 |
| | | treatments | | |
| 69 | White | Previously Received | N/A | Q3 2019 |
| | | Treatment | | |
| 54 | White | Previously Received | N/A | Q3 2019 |
| | | Treatment | | |
| 79 | White | Currently undergoing active | N/A | Q1 2020 |
| | | treatments | | |
| 1 | White | Treatment Naive | N/A | Q4 2019 |
| 65 | White | Currently undergoing active | N/A | Q1 2020 |
| | | treatments | | |
| 65 | White | Currently undergoing active | N/A | Q1 2021 |
| | | treatment | | |
| 31 | White | Previously Received | N/A | Q1 2020 |
| | | Treatment | | |
| 74 | White | Treatment Naive | N/A | Q1 2020 |
| 58 | White | Treatment Naive | N/A | Q1 2020 |
| 58 | White | Treatment Naive | N/A | Q1 2021 |
| 3 | White | | N/A | Q2 2017 |
| | Unknown | | in GPP, On Hold | Q1 2017 |
| 13 | White | | in DMX | Q3 2018 |
| 13 | White | Currently undergoing active | in DMX | Q3 2018 |
| | | treatments | | |
| 1 | Not reported | Currently undergoing active | N/A | Q3 2018 |
| | | treatments | | |
| 0 | Not reported | Treatment Naive | in DMX | Q4 2018 |
| 17 | White | Treatment Naive | N/A | Q3 2018 |
| 2 | White | Currently undergoing active | in DMX | Q4 2018 |
| | | treatments | | |
| 1 | Black or African American | Treatment Naive | N/A | Q1 2020 |
| 31 | Hispanic or Latino | Treatment Naive | N/A | Q2 2018 |
| 14 | Black or African American | Treatment Naive | N/A | Q3 2018 |
| 32 | White | Unknown | N/A | Q4 2017 |
| 16 | White | Unknown | in DMX | Q3 2018 |
| 43 | White | Unknown | in DMX | Q3 2018 |
| 35 | White | Unknown | in DMX | Q3 2018 |
| 66 | White | Unknown | N/A | Q4 2018 |
| 38 | Hispanic or Latino | Treatment Naive | N/A | Q2 2019 |
| 56 | White | Unknown | N/A | Q4 2018 |
| 64 | White | Unknown | N/A | Q4 2019 |
| 66 | White | Currently undergoing active | N/A | Q1 2020 |
| | | treatments | | |
| 66 | White | Currently undergoing active | N/A | Q1 2020 |
| | | treatments | | |
| | | | N/A | Q1 2018 |
| 61 | Asian | | N/A | Q4 2017 |
| 35 | White | Currently undergoing active | N/A | Q3 2019 |
| | | treatments | | |
| 47 | White | Previously received | N/A | Q1 2020 |
| | | treatment | | |
| 47 | White | Treatment Naive | N/A | Q1 2020 |
| 64 | White | Treatment Naive | N/A | Q1 2020 |
| 65 | White | Treatment Naive | N/A | Q1 2020 |
| 57 | White | Treatment Naive | N/A | Q1 2020 |
| 73 | White | Treatment Naive | N/A | Q4 2019 |
| 54 | Black or African American | Treatment Naive | N/A | Q1 2020 |
| 61 | White | Treatment Naive | N/A | Q1 2020 |
| | | | in GPP, On Hold | Q2 2016 |
| | | | in GPP, On Hold | Q2 2016 |
| | | | in GPP, On Hold | Q3 2016 |
| | | | N/A | Q4 2016 |
| | | | in GPP, On Hold | Q4 2016 |
| | | | N/A | Q2 2018 |
| | | | N/A | Q4 2017 |
| | | | N/A | Q1 2018 |
| | | | N/A | Q4 2017 |
| | | | N/A | Q2 2018 |
| | | | N/A | Q3 2018 |
| | | | N/A | Q4 2016 |
| | | | N/A | Q4 2016 |
| | | | N/A | Q1 2016 |
| | | | N/A | 2016 |
| | | | N/A | 2016 |
| | | | N/A | Q2 2017 |
| | | | N/A | Q1 2016 |
| | | | N/A | Q1 2016 |
| | | | N/A | Q2 2016 |
| | | | N/A | Q2 2015 |
| | | | N/A | Q2 2015 |
| | | | N/A | Q2 2018 |
| | | | N/A | Q4 2016 |
| | | | N/A | Q4 2016 |
| | | | N/A | Q2 2014 |
| | | | N/A | Q4 2018 |
| | | | N/A | Q4 2018 |
| | | | N/A | Q2 2015 |
| | | | N/A | Q2 2015 |
| | | | N/A | Q1 2016 |
| | | | N/A | Q2 2016 |
| | | | N/A | Q2 2016 |
| 15 | Black or African American | | N/A | Q2 2016 |
| 15 | Black or African American | | in DMX | Q2 2017 |
| 6 | White | | N/A | Q1 2015 |
| 12 | White | | Complete | Q4 2015 |
| 26 | White | | in DMX | Q1 2017 |
| | | | N/A | Q1 2015 |
| | | | N/A | Q1 2015 |
| | | | N/A | Q1 2015 |
| Unknown | | | N/A | Q3 2017 |
| 8 | White | | in GPP, On Hold | Q2 2015 |
| 17 | White | | Complete | Q4 2015 |
| | | | N/A | Q4 2015 |
| 26 | White | | in GPP, Active | Q4 2015 |
| 15 | White | | in GPP, Active | Q4 2015 |
| | | | in DMX | Q2 2016 |
| | | | N/A | Q4 2016 |
| 2 | White | | N/A | Q2 2016 |
| 2 | Hispanic or Latino | | in GPP, On Hold | Q2 2016 |
| | | | N/A | Q2 2016 |
| 13 | Black or African American | | in DMX | Q4 2016 |
| 16 | White | | in GPP, On Hold | Q4 2016 |
| 6 | White | | in DMX | Q4 2016 |
| 3 | White | | N/A | Q1 2017 |
| 0 | White | | in GPP, On Hold | Q1 2017 |
| 7 | White | | in GPP, On Hold | Q1 2017 |
| | | | N/A | Q2 2016 |
| | | | N/A | Q2 2016 |
| | | | N/A | Q3 2016 |
| | | | N/A | Q3 2016 |
| 60 | Hispanic or Latino | Treatment Naive | N/A | Q2 2021 |
| 60 | Hispanic or Latino | Treatment Naive | N/A | Q2 2021 |
| 66 | Black or African American | Treatment Naive | N/A | Q2 2021 |
| 66 | Black or African American | Treatment Naive | N/A | Q2 2021 |
| 66 | Black or African American | Treatment Naive | N/A | Q2 2021 |
| 47 | Asian | Previously received | N/A | Q2 2021 |
| | | treatment | | |
| 57 | White | Previously received | N/A | Q2 2021 |
| | | treatment | | |
| 57 | White | Previously received | N/A | Q2 2021 |
| | | treatment | | |
| 57 | White | Previously received | N/A | Q2 2021 |
| | | treatment | | |
| 57 | White | Previously received | N/A | Q2 2021 |
| | | treatment | | |
| 57 | White | Previously received | N/A | Q2 2021 |
| | | treatment | | |
| 60 | White | Previously received | N/A | Q2 2021 |
| | | treatment | | |
| 49 | Unknown | Treatment Naive | N/A | Q2 2021 |
| 49 | Unknown | Treatment Naive | N/A | Q2 2021 |
| 49 | Unknown | Treatment Naive | N/A | Q2 2021 |
| | Unknown | Unknown | N/A | Q2 2021 |
| | Unknown | Unknown | N/A | Q2 2021 |
| | Unknown | Unknown | N/A | Q2 2021 |
| | Unknown | Unknown | N/A | Q2 2021 |
| | Unknown | Unknown | N/A | Q2 2021 |
| 57 | White | Previously received | N/A | Q2 2021 |
| | | treatment | | |
| 57 | White | Previously received | N/A | Q2 2021 |
| | | treatment | | |
| 57 | White | Previously received | N/A | Q2 2021 |
| | | treatment | | |
| 83 | Not reported | Previously received | N/A | Q2 2021 |
| | | treatment | | |
| 58 | Unknown | Previously received | N/A | Q2 2021 |
| | | treatment | | |
| 58 | Unknown | Previously received | N/A | Q2 2021 |
| | | treatment | | |
| 58 | Unknown | Previously received | N/A | Q2 2021 |
| | | treatment | | |
| 58 | Unknown | Previously received | N/A | Q2 2021 |
| | | treatment | | |
| 58 | Unknown | Previously received | N/A | Q2 2021 |
| | | treatment | | |
| 10 | White | Treatment Naive | N/A | Q2 2021 |
| 10 | White | Treatment Naive | N/A | Q2 2021 |
| 10 | White | Treatment Naive | N/A | Q2 2021 |
| 10 | White | Treatment Naive | N/A | Q2 2021 |
| 10 | White | Treatment Naive | N/A | Q2 2021 |
| 10 | White | Treatment Naive | N/A | Q2 2021 |
| 10 | White | Treatment Naive | N/A | Q2 2021 |
| 10 | White | Treatment Naive | N/A | Q2 2021 |
| Unknown | Unknown | Unknown | N/A | Q2 2021 |
| Unknown | Unknown | Unknown | N/A | Q2 2021 |
| Unknown | Unknown | Unknown | N/A | Q2 2021 |
| Unknown | Unknown | Unknown | N/A | Q2 2021 |
| Unknown | Unknown | Unknown | N/A | Q2 2021 |
| Unknown | Unknown | Unknown | N/A | Q2 2021 |
| Unknown | Unknown | Unknown | N/A | Q2 2021 |
| 68 | White | Unknown | N/A | Q2 2021 |
| 34 | White | Unknown | N/A | Q2 2021 |
| 34 | White | Unknown | N/A | Q2 2021 |
| 65 | White | Treatment Naive | N/A | Q2 2021 |
| 78 | White | Currently undergoing active | N/A | Q2 2021 |
| | | treatments | | |

TABLE 3
Glossary of Terms for Table 2

| | |
|---|---|
| CCLF Model ID | |
| Arxspan ID, if applicable | The ID registered with the Cancer Dependency Map |
| CCLF Publication ID, if applicable | |
| Lineage | |
| Diagnosis | |
| Diagnosis Subtype | |
| Rare or Common Cancer Model? | |
| Verification method | The method used to confirm a cell line as tumor |
| Short term or Long term culture? | Short term passage = ~p3-p5; Long term passage = beyond |
| | passage 5 and higher, through the end of Expansion |
| Culture system | 2D adherent; 2D suspension; 2D mix (mix of adherent and |
| | suspension cells); 3D organoid cultured in matrigel |
| Culture media | |
| Pass Expansion? | After the cell line was verifed as tumor, did it successfully reach |
| | the end of Expansion? (Yes; No; In Progress; No attempt yet; |
| | On Hold) |
| Status in model derivation pipeline | Stages in the CCLF pipeline: Nursery, Nursery Complete, |
| | Expansion, Expansion Complete, On Hold, Terminated |
| Patient tumor tissue or PDX tumor tissue? | |
| HCMI model? | Is this cancer model a part of the Human Cancer Models |
| | Initiative? (Yes; No; TBD = model derivation still in progress; |
| | pending = awaiting documentation; N/A = model failed |
| | expansion) |
| Deposited at ATCC? | Has this cancer model been submitted to ATCC? (Yes; To be |
| | shipped; Pending, N/A) |
| Accessibility | Where this cell line is currently accessible: (1) Available at |
| | ATCC; (2) Deposited at ATCC; not yet available online; (3) |
| | Currently at CCLF |
| Patient tumor type | Primary; Secondary; Metastatic; Recurrent; Unknown |
| Gender | |
| Age at Collection | |
| Race or Ethnicity | |
| Treatment History | Patient treatment history at the time of sample collection |
| Status in DepMap | |
| Quarter model added to this list | The quarter this cancer model was confirmed to be “verified |
| | tumor” |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A combinatorial addressable array configured for high-throughput analysis of a sample comprising:
an addressable array configured to receive the sample and allocate the sample to a plurality of discrete locations across the addressable array,
wherein two or more of the discrete locations of the addressable array comprises at least two different culture conditions, and
wherein, for each of the at least two different culture conditions, there is at least two other discrete locations on the addressable array that each comprise only that culture condition.

2. The combinatorial addressable array of claim 1, wherein the at least two different culture conditions are each independently selected from the group consisting of: a culture media, a biological agent, a chemical agent, a pharmaceutical agent, a genetic modifying agent, a radioactive agent, a scaffold material, a culture type, a physical stress, a chemical stress, a biological stress, and a combination thereof.

3. The combinatorial addressable array of claim 2, wherein the cell culture media is a conditioned cell culture media.

4. The combinatorial addressable array of claim 1, wherein the two different culture conditions are each a cell culture media and wherein the cell culture medias are different from each other.

5. The combinatorial addressable array of claim 4, wherein one or both of the cell culture medias is/are a conditioned media.

6. The combinatorial addressable array of claim 5, wherein the condition media is conditioned media generated from a cancer cell line, a non-diseased cell line, a tumor organoid, a non-disease organoid, an engineered cell line, or a combination thereof.

7. The combinatorial addressable array of claim 1, wherein one or more of the discrete locations of the plurality of discrete locations on the addressable array comprises cells, tissue, an organoid, or a combination thereof.

8. The combinatorial addressable array of claim 7, wherein the cells, tissue, organoid, or any combination thereof are cancer cells, cancer tissue, a cancer organoid, or are generated from one or more cancer cells.

9. The combinatorial addressable array of claim 1, wherein the addressable array comprises a plurality of wells, one or more microfluidic channels, a 2D polymer, a 3D polymer, a gel, a planar surface, a non-planar surface, or any combination thereof.

10. A high-throughput method of empirically determining culture conditions effective to modify a biological sample, comprising:
culturing a biological sample having an initial characteristic state in one or more of the discrete locations on the combinatorial addressable array of claim 1; and
determining a change or no change in the initial state of a characteristic of the biological sample, wherein a change in the initial state of the characteristic identifies one or more conditions effective to modify the characteristic in the biological sample.

11. The method of claim 10, wherein determining a change or no change in the characteristic of the biological sample comprises performing gene sequencing, genome sequencing, a gene expression analysis, an epigenetic analysis, a cell phenotype analysis, a cell morphology analysis, a growth analysis, a differentiation analysis, a cell volume analysis, a cell viability analysis, a cell metabolism analysis, a cell communication or signal transduction analysis, a cell reproduction analysis, a cell response analysis, a cell production or secretion analysis, a cell function analysis or any combination thereof.

12. The method of claim 10, wherein the characteristic is growth, differentiation, proliferation, organoid formation, viability, cell death, apoptosis, cell product production, cell product secretion, gene expression, protein expression, epigenome state, metabolism, cell volume, cell size, cell state, cell type, cell subtype, cell morphology, or any combination thereof.

13. The method of claim 10, wherein the biological sample comprises a cell or cell population, a tissue, an organoid, or any combination thereof.

14. The method of claim 13, wherein the cell population is a heterogenous cell population or is a homogenous cell population.

15. The method of claim 10, wherein the biological sample comprises a cancer cell, a cancer tissue, a cancer organoid, or any combination thereof.

16. The method of claim 10, wherein the biological sample is cultured under two-dimensional culture conditions, three-dimensional culture conditions, suspension conditions, spheroid conditions, adherent conditions, aerobic conditions, anaerobic conditions, or any permissible combination thereof.

17. A cell culture condition effective to modify a characteristic of a biological sample during culture comprising:
a cell culture condition identified by performing a method as in claim 10.

18. A method of creating a cell line or organoid, the method comprising:
culturing a cell or cells isolated from a subject in a culture condition as in claim 17.

19. The method of claim 18, wherein the cell or cells forms an organoid, a spheroid, a cell suspension model, an adherent cell model, or a combination thereof.

20. The method of claim 18, wherein the cell or cells isolated from the subject is/are a cancer cell(s).

21. The method of claim 18, wherein culturing comprises passaging the cell or cells one or more times.

22. The method of claim 18, wherein culturing does not comprise passaging.

23. The method of claim 18, wherein culturing comprises expanding the cell or cells.

24. A computer-implemented method of training a statistical or machine learning model for determining culture conditions, predicting culture conditions, or both, effective for growth of a biologic sample, comprising:
collecting a set of sample culture parameters from a database to generate a collected set of sample culture parameters;
applying one or more transformations to each sample culture parameters to create a modified set of sample culture parameters;
creating a first training set comprising the collected set of sample culture parameters, the modified set of sample culture parameters, and a set of non-effective sample culture parameter results;
training a statistical model or a machine learning algorithm in a first stage using the first training set;
optionally creating a second training set for a second stage of training comprising the first training set and optionally, sample culture parameters that are incorrectly detected as effective sample culture parameters after the first stage of training; and
optionally training a neural network in a second stage using the second training set.

25. The computer-implemented method of claim 24, wherein the database comprises one or more of the following: one or more clinical annotations of biologic samples, treatment response history of biologic samples, cell culture condition response of biologic samples, optimal parameters for biologic samples, processing method history of biologic samples, phenotype of biologic samples, genomic profile of biologic samples, epigenomic profile of biologic samples, biologic sample source annotations, or any combination thereof.

26. The computer-implemented method of claim 25, wherein the one or more clinical annotations is/are any one or more of those set forth in Appendix A.

27. The computer-implemented method of claim 24, wherein the statistical model or the machine learning algorithm is configured as a neural network, a decision tree, a support vector machine, a linear regression, a logistical regression, a random forest, a gradient boosted trees, a naive bayes, a nearest neighbor, a k-means clustering, a t-SNE, a principal component analysis, an association rule, a Q-learning, a temporal difference, a Monte-Carlo tree search, an asynchronous actor-critic agents, or any permissible combination thereof.

28. A computer-implemented method for determining culture conditions, predicting culture conditions, or both, effective for growth of a biologic sample, comprising:
receiving biologic sample data;
optionally applying one or more filters to the biologic sample data;
using the received biologic sample data or filtered biologic sample data as input and applying a one or more classifiers to determine and/or predict one or more effective biologic sample biologic sample culture conditions based on a computer-accessible database, trained statistical or machine-learning model trained to predict effective biologic sample culture conditions based on the one or more classifiers, a statistical data analysis methodology, or any combination thereof.

29. The computer-implemented method of claim 28, wherein the one or more determined effective biological sample culture condition(s), predicted effective biologic sample culture condition(s), or both, are passed through one or more additional filters to further optimize the determined effective biologic sample culture conditions, predicted effective biologic sample culture conditions, or both.

30. The computer-implemented method of claim 29, further comprising
applying one or more additional classifiers to the one or more determined effective biologic sample culture conditions, predicted effective biologic sample culture conditions, or both;
applying one or more additional classifiers to the one or more further optimized determined effective biologic sample culture conditions, one or more further optimized predicted effective culture conditions, or both; or
both;
to determine, predict, or both one or more effective biologic sample biologic sample culture conditions based on the computer-accessible database, trained machine-learning model trained to predict effective biologic sample culture conditions based on the one or more additional classifiers, or both.

31. The computer-implemented method of claim 28, wherein the trained statistical or machine-learning model is produced by a computer-implemented method of training a statistical or machine learning model for determining culture conditions, predicting culture conditions, or both, effective for growth of a biologic sample, comprising:
collecting a set of sample culture parameters from a database to generate a collected set of sample culture parameters;
applying one or more transformations to each sample culture parameters to create a modified set of sample culture parameters;
creating a first training set comprising the collected set of sample culture parameters, the modified set of sample culture parameters, and a set of non-effective sample culture parameter results;
training a statistical model or a machine learning algorithm in a first stage using the first training set;
optionally creating a second training set for a second stage of training comprising the first training set and optionally, sample culture parameters that are incorrectly detected as effective sample culture parameters after the first stage of training; and
optionally training a neural network in a second stage using the second training set.

32. The computer-implemented method of claim 24, wherein the biologic sample data is received from a user input, one or more sensors, one or more detection devices, one or more sample characteristic measurement devices, one or more sample characteristic analysis devices, a database, or any combination thereof.

33. The computer-implemented method of claim 28, wherein the biological sample is contained in an addressable array as in claim 1.

34. A computer-implemented method to determine, predict, or both culture conditions effective growth for growth of a biologic sample, comprising:
receiving data of one or more parameters from the biologic sample in a format usable by a computing device;
executing processing logic configured to generate feature data from the received data, filter the received data, filter the feature data, process the feature data, process the received data, or any combination thereof with one or more trained machine learning models that is/are trained to predict effective biologic sample culture conditions based on the received data, feature data, or both; and
executing processing logic configured to cause a list of the effective biologic sample culture conditions to be displayed via an electronic display, transmitted to a user interface program, be saved to a non-transitory computer readable memory, or any combination thereof.

35. The computer-implemented method of claim 34, wherein at least one of the one or more trained statistical or machine learning models are produced by a computer-implemented method of training a statistical or machine learning model for determining culture conditions, predicting culture conditions, or both, effective for growth of a biologic sample, comprising:
collecting a set of sample culture parameters from a database to generate a collected set of sample culture parameters;
applying one or more transformations to each sample culture parameters to create a modified set of sample culture parameters;
creating a first training set comprising the collected set of sample culture parameters, the modified set of sample culture parameters, and a set of non-effective sample culture parameter results;
training a statistical model or a machine learning algorithm in a first stage using the first training set;
optionally creating a second training set for a second stage of training comprising the first training set and optionally, sample culture parameters that are incorrectly detected as effective sample culture parameters after the first stage of training; and
optionally training a neural network in a second stage using the second training set.

36. The computer-implemented method of claim 34, wherein the data of one or more parameters is received from user input, one or more sensors, one or more detection devices, one or more sample characteristic measurement devices, one or more sample characteristic analysis devices, a database, or any combination thereof.

37. The computer-implemented method of claim 34, wherein the biological sample is contained in an addressable array as in claim 1.

38. A non-transitory computer readable medium comprising computer-executable instructions recorded thereon for causing a computer to perform the method of claim 24.

39. A non-transitory computer readable medium comprising computer-executable instructions recorded thereon for causing a computer to perform the method of claim 34.

40. A system comprising:
non-transitory computer-readable medium; and
a processor configured to execute instructions stored on the non-transitory computer readable medium which, when executed, cause the processor to perform the method of claim 24.

41. A system comprising:
non-transitory computer-readable medium; and
a processor configured to execute instructions stored on the non-transitory computer readable medium which, when executed, cause the processor to perform the method of claim 34.
